(19) United States
(12) Patent Application Publication
(10) Pub. No.: US 2024/0254521 A1
(43) Pub. Date: Aug. 1, 2024

(54) ENZYMES, HOST CELLS, AND METHODS FOR PRODUCTION OF ROTUNDONE AND OTHER TERPENOIDS

(71) Applicant: MANUS BIO INC., Waltham, MA (US)

(72) Inventors: Stephen SARRIA, Waltham, MA (US); Yiying ZHENG, Waltham, MA (US); Morgan FETHEROLF, Waltham, MA (US); Liwei LI, Waltham, MA (US); Ajikumar PARAYIL-KUMARAN, Waltham, MA (US); Christine Nicole S. SANTOS, Waltham, MA (US); Jason Eric DONALD, Waltham, MA (US)

(21) Appl. No.: 18/560,260

(22) PCT Filed: May 11, 2022
(86) PCT No.: PCT/US2022/028782

§ 371 Date: Nov. 10, 2023

Related U.S. Application Data
(60) Provisional application No. 63/186,949, filed on May 11, 2021.

(51) Int. Cl. C12P 7/26; C12N 9/02; C12N 9/04; C12N 9/88; C12N 15/52; C12P 5/00

(52) U.S. Cl. CPC C12P 7/26; C12N 9/0006; C12N 9/0042; C12N 9/0073; C12N 9/88; C12N 15/52; C12P 5/007; C12Y 101/01001; C12Y 106/02004; C12Y 114/13078; C12Y 402/03087

(57) ABSTRACT

The present disclosure in various aspects provides engineered enzymes and encoding polynucleotides, as well as host cells and methods for making rotundone and other terpenoids. For example, in various aspects, the invention provides engineered α-Guaiene Synthase (αGS) and Guaiene Oxidase (GO) enzymes that increase biosynthesis of rotundone from farnesyl diphosphate, and in certain embodiments substantially reduce biosynthesis of side products such as α-Bulnesene or oxygenated side products. In still other aspects, the invention provides engineered terpene synthase enzymes for directing biosynthesis toward a desired product, to thereby improve product profiles and/or product titers from terpene synthase reactions.

### BACKGROUND

Rotundone is an oxygenated sesquiterpene (sesquiterpenoid) that is responsible for a pleasing spicy, ‘peppery’ aroma in various plants, including grapes (especially syrah or shiraz, mourvèdre, durif, vespolina, and grüner veltliner varietals), and a large number of herbs and spices, such as, e.g., black and white pepper, oregano, basil, thyme, marjoram, and rosemary. Given its aroma, rotundone is an attractive molecule for applications in fragrances and flavors.

α-Guaiene is the precursor to (−)-rotundone. α-Guaiene is a sesquiterpene hydrocarbon found in oil extracts from various plants and is converted to (−)-rotundone (“rotundone”) by aerial oxidation or enzymatic transformation.

Given the commercial value of rotundone, cost effective, scalable, and/or sustainable processes for its production are desired.

### SUMMARY OF THE DISCLOSURE

The present disclosure in various aspects provides engineered enzymes and encoding polynucleotides, as well as host cells and methods for making rotundone and other terpenoids. For example, in various aspects, the invention provides engineered α-Guaiene Synthase (αGS) and Guaiene Oxidase (GO) enzymes that increase biosynthesis of rotundone from farnesyl diphosphate, and in certain embodiments substantially reduce biosynthesis of side products such as α-Bulnesene or oxygenated side products. In still other aspects, the invention provides engineered terpene synthase enzymes (e.g., Class I Terpene Synthase enzymes) for directing biosynthesis toward a desired product (“a target terpenoid”), to thereby improve product profiles and/or product titers from terpene synthase reactions.

In one aspect, the invention provides host cells and methods for producing rotundone. The method comprises providing a host cell producing farnesyl diphosphate, and expressing a heterologous rotundone biosynthesis pathway, the rotundone biosynthesis pathway comprising an α-Guaiene Synthase (αGS) and a α-Guaiene Oxidase (αGO). In various embodiments, the αGS comprises an amino acid sequence having at least 70% sequence identity to amino acids 258 to 548 of SEQ ID NO: 1 (which comprises the enzyme active site), and/or the αGO comprises an amino acid sequence having at least 70% sequence identity to the amino acid sequence of SEQ ID NO: 6. The host cell is cultured under conditions to allow for rotundone production, and rotundone is recovered from the culture. In various embodiments, the microbial cells can synthesize rotundone product from any suitable carbon source. In some embodiments, the specificity of the αGS enzyme enables production of α-Guaiene at high titers with lower levels of terpenoid side products, as compared to the enzyme of SEQ ID NO: 1. That is, the αGS comprises one or more amino acid modifications with respect to SEQ ID NO: 1 that increase production of α-Guaiene relative to side products such as α-Bulnesene. Further, the αGO may comprise one or more amino acid modifications with respect to SEQ ID NO: 6 that improve production of rotundone and/or rotundol from α-Guaiene, relative to the enzyme defined by SEQ ID NO: 6. Further, in some embodiments, the microbial host cell may further express one or more alcohol dehydrogenase (ADH) enzymes, where the ADH converts one or more alcohol intermediates, produced by the reaction of α-Guaiene with αGO, to rotundone.

Terpene synthase enzymes can generate multiple products with the guaiene skeleton from FPP with varied amounts of α-Guaiene produced by different TPS enzymes. In some embodiments, the αGS engineered as described herein produces predominantly α-Guaiene as the product from FPP substrate.

As demonstrated herein, one or more amino acid modifications can be made to the αGS that stabilize a carbocation at C2 or C6 of the catalytic intermediate to direct catalysis toward α-Guaiene, and/or to destabilize a carbocation at C7 of the catalytic intermediate to direct catalysis away from the major side product α-Bulnesene. For example, one or more amino acid modifications to the αGS can stabilize the carbocation at C2 or C6 by adding a cation-π interaction between an aromatic side chain and a carbocation at C2 or C6 of the catalytic intermediate. One or more amino acid modifications may also destabilize a carbocation at C7 by removing an interaction between an aromatic or aliphatic side chain and a carbocation at C7. During catalysis, deprotonation of a neighboring carbon (neighboring the carbocation) produces the cyclized product.

In various embodiments, the αGS comprises amino acid substitutions at one or more positions selected from 21, 269, 273, 290, 293, 296, 325, 375, 400, 407, 443, 447, 448, 545 with respect to SEQ ID NO: 1. For example, the αGS may comprise one or more amino acid substitutions selected from Y21F, G269S, M273I, N290T, N290A, 1293F, T296V, E325T, S375A, 1400L, 1400V, F407L, Y443L, Y443V, Y443F, L447V, Q448V, and A545P with respect to SEQ ID NO: 1. In some embodiments, the αGS comprises the amino acid sequence of SEQ ID NO: 28, 31, or 32, or comprises the amino acid sequence of residues 258 to 548 of SEQ ID NO: 28, 31, or 32.

Accordingly, in one aspect of this disclosure, the invention provides engineered αGS enzymes (and encoding polynucleotides and host cells comprising the same). The αGS enzymes are engineered for productivity and/or improved product profile toward α-Guaiene, and away from the major side product α-Bulnesene. In various embodiments, the αGS enzyme comprises an amino acid sequence that has at least about 90% sequence identity with amino acids 258 to 548 of SEQ ID NO: 28, wherein the α-Guaiene Synthase comprises (i.e. retains) a Phenylalanine at the position corresponding to position 293 of SEQ ID NO: 28, and optionally retains a non-aromatic residue at the position corresponding to position 407 of SEQ ID NO: 28. For example, the amino acid at the position corresponding to position 407 of SEQ ID NO: 28 is not Phenylalanine.

In some embodiments, the αGS enzyme comprises an amino acid sequence that has at least about 90% sequence identity with amino acids 258 to 548 of SEQ ID NO: 31 or SEQ ID NO: 32, wherein the α-Guaiene Synthase comprises (i.e. retains) a Phenylalanine at the position corresponding to position 293 of SEQ ID NO: 31 or 32, and optionally retains a non-aromatic residue at the position corresponding to position 407 of SEQ ID NO: 31 or 32. For example, the amino acid at the position corresponding to position 407 of SEQ ID NO: 31 or 32 is not Phenylalanine.

Accordingly, one aspect of the disclosure provides engineered αGO enzymes (and encoding polynucleotides and host cells comprising the same). The αGO enzyme is engineered for productivity and/or improved product profile toward rotundol or rotundone. In some embodiments, the αGO enzyme comprises an amino acid sequence that has at least about 90% sequence identity to SEQ ID NO: 30, wherein the αGO comprises (i.e., retains) the amino acid at positions selected from one or more (e.g., 2, 3, 4, 5, or all) of 235, 238, 318, 371, 440, 489, 490, and 495 of SEQ ID NO: 30. In some embodiments the αGO comprises substitutions at positions 184, 389, and 501 with respect to SEQ ID NO: 30. For example, the αGO may comprise the amino acid sequence of SEQ ID NO: 33

In another aspect, the present disclosure provides a method for making rotundone. The method comprises providing a microbial host cell as disclosed herein. The microbial host cell expresses an αGS and/or an αGO enzyme, as described herein. Cells expressing an αGO enzyme can be used for bioconversion of α-Guaiene to rotundone using whole cells or cell extracts or purified recombinant enzyme. Cells expressing an αGO enzyme and an αGS enzyme can produce rotundone from any suitable carbon source. In some embodiments, the microbial host cell further expresses one or more alcohol dehydrogenase (ADH) enzymes, such as those disclosed herein. Cells expressing ADH enzymes can convert alcohol intermediates produced by the αGO reaction into rotundone.

As exemplified and demonstrated herein with regard to the engineering of αGS, another aspect of the invention provides methods for engineering terpene synthase enzymes (and methods of using the same) by modifying the amino acid sequence to favor certain catalytic intermediates over others. For example, the method may comprise providing a terpene synthase amino acid sequence (e.g., a Class I Terpene Synthase amino acid sequence), where the terpene synthase is capable of catalyzing cyclization of a prenyl diphosphate to produce a target cyclic terpenoid and one or more non-target cyclic terpenoids through deprotonation of a series of cyclic carbocation intermediates. In various embodiments, synthesis of the target cyclic terpenoid versus non-target cyclic terpenoids will be based on the position of deprotonation of the carbocation intermediate.

The terpene synthase amino acid sequence will comprise one or more amino acid modifications (with respect to a wild type or parent terpene synthase enzyme) so as: to position an aromatic side chain to stabilize a carbocation catalytic intermediate (via a cation-π interaction) that deprotonates to the target cyclic terpenoid; and/or to remove or shift one or more aromatic or aliphatic side chains to destabilize a carbocation intermediate that deprotonates to at least one non-target cyclic terpenoid. These modifications alter the product profile toward the target terpenoid, and away from non-target terpenoid(s). The engineered terpene synthase enzyme may be recombinantly produced and may be heterologously expressed in microbial cells for microbial production of the desired compound as described herein.

Other aspects and embodiments of the disclosure will be apparent to the skilled person in view of the following detailed disclosure.

### BRIEF DESCRIPTION OF THE FIGURES

FIG. 1A illustrates a proposed mechanism for cyclization of FPP to α-Guaiene by terpene synthase, along with major side product α-Bulnesene, and other side products. Proposed catalytic intermediates (INT1-7) are shown. FIG. 1B illustrates a biosynthetic pathway for the production of rotundone. Farnesyl diphosphate is converted to α-Guaiene by an α-Guaiene Terpene Synthase (αGTPS or αGS) enzyme, and α-Guaiene is converted to (−)-rotundone by an α-Guaiene Oxidase (αGOX or αGO).

FIG. 2 illustrates the active site of a homology model of αGS0 (SEQ ID NO: 1). Three amino acid residues (S375A, F407L, and Y443L) were identified during Round 1 engineering. The substitution F407L may disfavor the stabilization of INT4 and push the enzyme to favor INT5 for higher α-Guaiene production. Mutation S375A may disfavor the deprotonation of INT5 to α-Bulnesene, and consequently favor the deprotonation INT5 to α-Guaiene process.

FIG. 3 illustrates the active site of a homology model of αGS1 (SEQ ID NO: 2). The substitution N290T was identified during Round 2 engineering.

FIG. 4 compares α-Guaiene titer (gray bars) and % α-Guaiene (line) produced by fermentation of E. coli strains expressing αGS1 (SEQ ID NO: 2) or αGS2 (SEQ ID NO: 3) in 96 well plates for 72 hours.

FIG. 5 illustrates the active site of a homology model of αGS3 (SEQ ID NO: 4). Two amino acid substitutions (T290A) and (1293F) were identified during Round 3 engineering. The I293F substitution may favor stabilization of INT5 with cation-π interaction and support higher α-Guaiene production.

FIG. 6 compares α-Guaiene titer (gray bars) and % α-Guaiene (line) produced by fermentation of E. coli strains expressing αGS1 (SEQ ID NO: 2) or αGS3 (SEQ ID NO: 4) in 96 well plates for 72 hours.

FIG. 7 illustrates the active site of a homology model of αGS4 (SEQ ID NO: 5). Three amino acid substitutions (M273L, I400L, and L447V) were identified during Round 4 engineering. Substitution M273L may alter the distance of C helix to INT5 and favor the deprotonation of INT5 to α-Guaiene.

FIG. 8 compares α-Guaiene titer (gray bars) and % α-Guaiene (line) produced by fermentation of E. coli strains expressing αGS3 (SEQ ID NO: 4) or αGS4 (SEQ ID NO: 5) in 96 well plates for 72 hours.

FIG. 9 illustrates a homology model of GO (SEQ ID NO: 6). Two amino acid substitutions (M235R and E318L) were identified during Round 1 engineering. Substitution E318L may bring the substrate closer to the heme reaction center to favor the major products rotundone and rotundol.

FIG. 10 shows the results of Round 5 of αGS engineering. In vivo production of α-Guaiene with αGS4 and lead mutant αGS5 is shown. Fermentation was performed in a 96 well plate for 72 hours.

FIG. 11 shows a comparison of αGS1 and αGS5. In vivo production of α-Guaiene with α-GS1 and α-GS5 is shown. Fermentation was performed in a 96 well plate for 72 hours.

FIG. 12 shows generational α-GS as a function of α-Guaiene percent of the total products. In vivo production of α-Guaiene are shown from an engineered E. coli strain expressing α-GS0 through α-GS5. Fermentation was performed in a 96 well plate for either 48 or 72 hours.

FIG. 13 illustrates a homology model of GO1 (SEQ ID NO: 7). Two amino acid substitutions (I238A and S320T) were identified during Round 2 engineering.

FIG. 14 shows GO activity on α-Guaiene and α-Bulnesene substrates. In vivo production of rotundol, rotundone, and other oxygenated products are shown from an engineered E. coli strain co-expressing GO5, α-GS5, a CPR (SEQ ID NO: 20), and an ADH (SEQ ID NO: 10). Fermentation was performed in a 96 well plate for 72 hours.

FIG. 15 illustrates a GS0 homology model, with secondary structures annotated according to Table 8.

FIG. 16 illustrates formation of desired product α-Guaiene and main side product α-Bulnesene by quenching different intermediates.

FIG. 17 illustrates the computed reaction pathway and potential energy profile of proposed reaction mechanism for the formation of α-Guaiene. Potential energies (in kcal/mol) at the B3LYP/6-31G* level at gas phase are shown. All calculated energies are relative to (E,Z)-farnesyl cation.

FIG. 18 shows the superimposed structures of INT4, INT5, and INT6. All structures are optimized at the B3LYP/level.

FIG. 19A and FIG. 19B illustrate stabilization of INT5 with (FIG. 19A) a benzene group (e.g., Phenylalanine side chain) versus (FIG. 19B) propane (e.g., similar to a Leucine side chain). FIG. 19A shows B3LYP optimized complex of INT5 and benzene. The distance between C6 of INT5 to the center of the benzene ring is 4.2 Ang. The formation of this complex releases 6.3 kcal/mol of energy. FIG. 19B shows B3LYP optimized complex of INT5 and propane. The distance between C6 of INT5 to C2 of propane is 5.0 Ang. The formation of this complex releases 1.5 kcal/mol of energy.

FIG. 19C illustrates the region selected for stabilization using cation-π interactions.

FIG. 20 is a stereoview showing the bottom of the enzyme pocket of GS0.

FIG. 21 illustrates a mechanism of cation-π stabilized intermediates in the GS pocket.

FIG. 22 illustrates three important residues for GS engineering.

FIG. 23(A-C) shows the position alignment for (A) F407, (B) 1293, and (C) M273 based on αGS0.

FIG. 24 is a table listing aromatic residues in the pocket for various sesquiterpene cyclase enzymes, and their location. TEAS is the 5-epi-aristolochene synthase from Nicotiana tabacum, a model sesquiterpene cyclase.

FIG. 25 shows the results of Round 6 of αGS engineering. In vivo production of α-Guaiene with αGS4 and lead mutant αGS5 is shown. Fermentation was performed in a 96 well plate for 72 hours.

FIG. 26 compares the GS activity of α-GS6 and α-GS7 to produce α-Guaiene and α-Bulnesene. The α-Guaiene, α-bulnesene and total cyclized products from fermentations by engineered E. coli strains expressing α-GS6 or α-GS7 were plotted. Fermentation was performed in a 96 well plate for 72 hours.

FIG. 27 shows the results of Round 6 of GO engineering. Shown is the in vivo production of rotundol-1, rotundol-2, rotundone, and total oxygenated products from engineered E. coli strains co-expressing GO5 or GO6 with α-GS7 (SEQ ID NO: 32), a CPR (SEQ ID NO: 20), and an ADH (SEQ ID NO: 10). Fermentations were performed in a 96 well plate for 72 hours.

FIG. 28 is a table showing in vivo production of rotundol and rotundone containing various CPR homologs (SEQ ID NOs: 21 and 34 to 36) in ΔCPR strains co-expressing α-GS (SEQ ID NO: 28), GO (SEQ ID NO: 30) in comparison with similar strain expressing the CPR of SEQ ID NO: 20. Fermentation was performed in a 96 well plate for 72 hours.

FIG. 29 is a table showing in vivo production of rotundol and rotundone with bacterial strains expressing various ADH homologs and co-expressing α-GS5 (SEQ ID NO: 28), GO5 (SEQ ID NO: 30) and SEQ ID NO: 20, in comparison with similar strain expressing ADH of SEQ ID NO: 10. Fermentation was performed in a 96 well plate for 72 hours.

### DETAILED DESCRIPTION

The present disclosure in various aspects provides engineered enzymes and encoding polynucleotides, as well as host cells, and methods for making rotundone and other terpenoids. For example, in various aspects, the invention provides engineered α-Guaiene Synthase (αGS) and Guaiene Oxidase (GO) enzymes that improve biosynthesis of rotundone from farnesyl diphosphate, and in certain embodiments improve the product profile to substantially reduce biosynthesis of side products such as α-Bulnesene or oxygenated side products. In still other aspects, the invention provides engineered terpene synthase enzymes for directing terpene biosynthesis toward a desired product, to thereby improve product profiles and/or product titers from terpene synthase reactions.

In one aspect, the invention provides host cells and methods for producing rotundone. The method comprises providing a host cell producing farnesyl diphosphate, and expressing a heterologous rotundone biosynthesis pathway, the rotundone biosynthesis pathway comprising an α-Guaiene Synthase (αGS) and a Guaiene Oxidase (GO). In various embodiments, the αGS comprises an amino acid sequence having at least 70% sequence identity to amino acids 258 to 548 of SEQ ID NO: 1 (which comprises the enzyme active site), and/or the GO comprises an amino acid sequence having at least 70% sequence identity to the amino acid sequence of SEQ ID NO: 6. The host cell is cultured under conditions to allow for rotundone production, and rotundone is recovered from the culture. In various embodiments, the microbial cells can synthesize rotundone product from any suitable carbon source. In some embodiments, the specificity of the α-GS enzyme enables production of α-Guaiene at high titers with fewer terpenoid side products, as compared to the enzyme of SEQ ID NO: 1. That is, the αGS comprises one or more amino acid modifications with respect to SEQ ID NO: 1 that increase production of α-Guaiene relative to side products such as α-Bulnesene. Further, the αGO may comprise one or more amino acid modifications with respect to SEQ ID NO: 6 that improve production of rotundone and/or rotundol from α-Guaiene, relative to the enzyme defined by SEQ ID NO: 6. Further, in some embodiments, the microbial host cell may further express one or more alcohol dehydrogenase (ADH) enzymes, where the ADH converts one or more alcohol intermediates, produced by the reaction of α-Guaiene with GO, to rotundone.

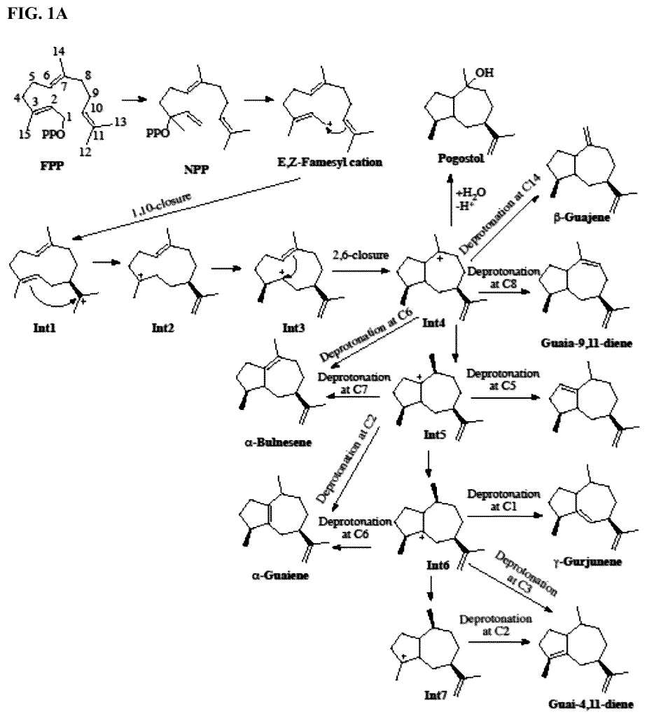

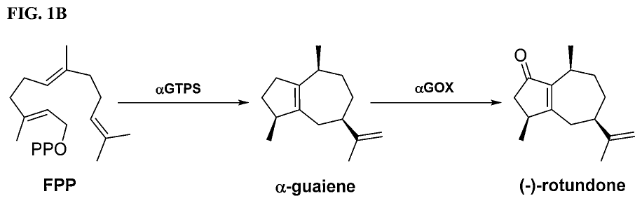

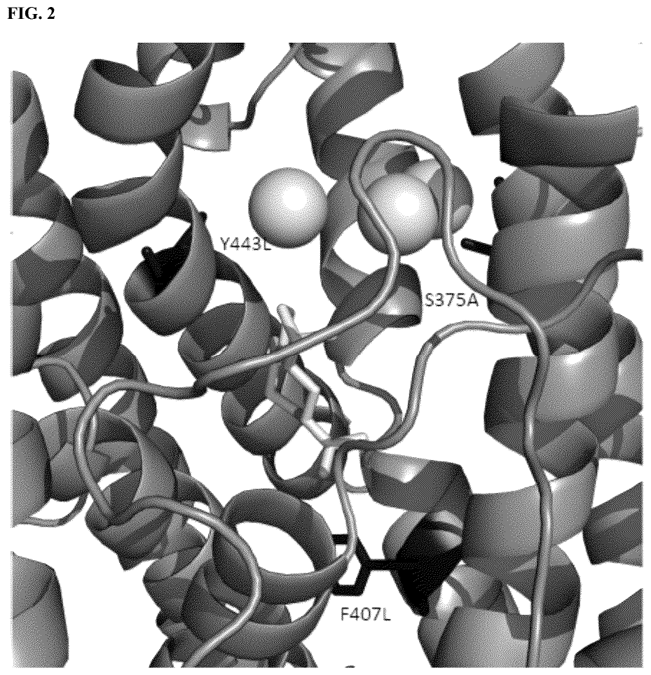

A biosynthetic mechanism for α-Guaiene (including proposed catalytic intermediates and side products) is shown in FIG. 1A. The C15 sesquiterpene precursor substrate farnesyl diphosphate (FPP) is cyclized to α-Guaiene by an α-Guaiene terpene synthase enzyme (αGS). This cyclization step can produce various other cyclized products, and α-Bulnesene is the major side product. The α-Guaiene is then oxidized to rotundone via an αGO enzyme. See FIG. 1B. The production of the ketone moiety in α-Guaiene resulting in rotundone can proceed directly, or can alternatively proceed through alcohol intermediates, with either stereochemistry of the alcohol intermediate, i.e., (2R)-rotundol or (2S)-rotundol.

The αGS enzyme is a terpene synthase enzyme (TPS). TPS enzymes are responsible for the synthesis of the terpene molecules from two isomeric 5-carbon precursor building blocks, leading to 5-carbon isoprene, 10-carbon monoterpenes, 15-carbon sesquiterpenes and 20-carbon diterpenes. The structures and functions of TPS enzymes are described in Chen et al., The Plant Journal, 66: 212-229 (2011). Tobacco 5-epi-aristolochene synthase, a terpene synthase, has been described along with structural coordinates, including key active site coordinates. These structural coordinates can be used for constructing homology models of TPS enzymes, which are useful for guiding the engineering of TPS enzymes with improved specificity and/or productivity. See U.S. Pat. Nos. 6,645,762, 6,495,354, and 6,645,762, which are hereby incorporated by reference in their entireties.

TPS enzymes can generate multiple products with the guaiene skeleton from FPP with varied amounts of α-Guaiene produced by different TPS enzymes. In some embodiments, the αGS engineered as described herein produces predominantly α-Guaiene (e.g., greater than 50%) as the product from FPP substrate. In some embodiments, the αGS produces greater than about 75%, or greater than about 80%, or greater than about 85%, or greater than about 90% α-Guaiene as the product from FPP. Enzyme specificity can be determined in host microbial cells producing FPP and expressing the α-Guaiene synthase, followed by chemical analysis of total terpenoid products.

In various embodiments, the αGS comprises an amino acid sequence having at least about 80%, or at least about 85%, or at least about 90%, or at least about 95%, or at least about 97% sequence identity, or at least about 98% sequence identity to amino acids 258 to 548 of SEQ ID NO: 1. This C-terminal portion of the enzyme contains the active site, and as disclosed herein, changes in this region can impact catalytic activity and product profiles. In various embodiments, the αGS comprises an amino acid sequence having at least about 80%, or at least about 85%, or at least about 90%, or at least about 95%, or at least about 97% sequence identity to the full sequence of SEQ ID NO: 1. In some embodiments, the αGS comprises an amino acid sequence having at least 98% sequence identity to SEQ ID NO: 1.

The similarity of nucleotide and amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST protein searches may be performed with the BLASTP program, score=50, word length=3. To obtain gapped alignments for comparative purposes, Gapped BLAST is utilized as described in Altschul et al (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

In various embodiments, the αGS comprises one or more amino acid substitutions with respect to SEQ ID NO: 1 within positions 258 to 548. As described herein, mutations in this region can impact product titers and product profile. In some embodiments, the αGS comprises from 2 to 20 or from 2 to 10 amino acid substitutions with respect to SEQ ID NO: 1 within positions 258 to 548, or within positions 269 to 500, where the amino acid substitutions improve α-Guaiene titer or product profile, with respect to the titer and product profile generated with the enzyme of SEQ ID NO: 1.

In some embodiments, modifications to the αGS are informed by construction of a homology model. The homology model can be based on structural coordinates from Nicotiana tabacum 5-epi-aristolochene synthase. See, U.S. Pat. Nos. 6,645,762, 6,495,354, and 6,645,762, which are hereby incorporated by reference in their entireties. In some embodiments, the amino acid modifications to the αGS can be selected to improve one or more of: enzyme productivity, selectivity for the desired substrate and/or product, stability, temperature tolerance, and expression in microbial host cells. In some embodiments, the αGS comprises one or more substitutions in a secondary structure element selected from the G2, D, J, and C helices, which form part of the active site (See Table 13). For example, at least one substitution of the αGS can be on the D helix, which can be an aromatic residue such as phenylalanine. For example, amino acid substitutions can be selected to position the center of a phenylalanine side chain (benzyl ring) within about 3 to 6 Ang of C2 of INT6 or C6 of INT5 (See FIG. 1A). Stabilization of the INT5 or INT6 carbocation (e.g., relative to INT4 carbocation) with cation-π interactions shifts the product profile dramatically toward α-Guaiene, and away from the major side product α-Bulnesene. In these or other embodiments, at least one substitution of the αGS is on the G2 helix, which can include a substitution to remove an aromatic side chain (e.g., phenylalanine) or an aliphatic side chain from the vicinity of (e.g., a distance of at least 5 or 6 Ang from) the INT4 carbocation. These modifications can destabilize this intermediate relative to INT5 and INT6.

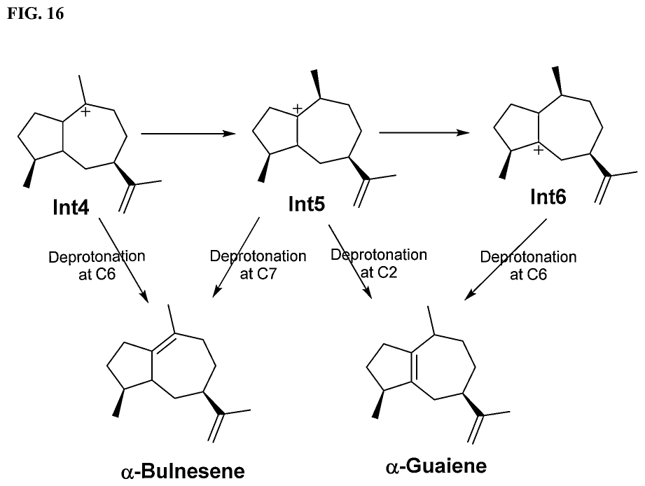

As demonstrated herein, one or more amino acid modifications can be made to the αGS that stabilize a carbocation at C2 or C6 of the catalytic intermediate to direct catalysis toward α-Guaiene, and/or to destabilize a carbocation at C7 of the catalytic intermediate to direct catalysis away from the major side product α-Bulnesene. For example, one or more amino acid modifications to the αGS can stabilize the carbocation at C2 or C6 by adding a cation-π interaction between an aromatic side chain and a carbocation at C2 or C6 of the catalytic intermediate. One or more amino acid modifications may also destabilize a carbocation at C7 by removing an interaction between an aromatic or aliphatic side chain and a carbocation at C7. Numbering of carbons of the intermediates is based on the numbering for FPP (See FIG. 1A). During catalysis, deprotonation of a neighboring carbon (neighboring the carbocation) produces the cyclized product, as shown in FIG. 16.

In some embodiments, amino acid substitutions include one or more amino acids having side chains within a distance of about 12 Ang., or within about 10 Ang., or within about 7 Ang. of the closest atom of the substrate or catalytic intermediate, or within a distance of about 12 Ang., or within about 10 Ang., or within about 7 Ang. of the carbocation of INT4, INT5, and/or INT 6. In these or other embodiments, amino acid substitutions shift the distance or geometries of these residues with respect to the substrate or intermediate (or carbocation thereof).

In various embodiments, the αGS comprises one or more substitutions at positions selected from 290, 325, 407, 499, 495, 341, 273, 375, 443, 447, and 294 with respect to SEQ ID NO: 1, and which improve α-Guaiene titer or percent α-Guaiene. For example, the αGS may comprise at least two, at least three, or at least four amino acid substitutions with respect to SEQ ID NO: 1 at positions selected from 290, 325, 407, 499, 495, 341, 273, 375, 447, and 294.

In various embodiments, the αGS comprises one or more amino acid modifications with respect to SEQ ID NO: 1, and which improve the α-Guaiene titer or percent α-Guaiene. For example, the αGS comprises one or more substitutions with respect to SEQ ID NO: 1 selected from S375A, F407L, and Y443L. In some embodiments, the αGS comprises the amino acid sequence of SEQ ID NO: 2.

In some embodiments, the αGS comprises the amino acid sequence of SEQ ID NO: 2, optionally with from 1 to 20, or from 1 to 10, or from 1 to 5, or from 1 to 3 amino acid modifications independently selected from substitutions, deletions, and insertions which improve α-Guaiene titer or percent α-Guaiene with respect to SEQ ID NO: 2. In some embodiments, the αGS comprises from 2 to 20 or from 2 to 10 amino acid substitutions with respect to SEQ ID NO: 2 within positions 258 to 548. For example, the αGS may comprise one or more amino acid substitutions with respect to SEQ ID NO: 2 at positions selected from 290, 325, 499, 495, 341, 273, 447, 294, 439, 504, 369, and 206, and which improve α-Guaiene titer or percent α-Guaiene with respect to the enzyme of SEQ ID NO: 2.

In some embodiments, the αGS comprises one or more amino acid modifications with respect to SEQ ID NO: 2 that are selected from Table 1, and which improve α-Guaiene titer or percent α-Guaiene. For example, the αGS in some embodiments comprises the substitution N290T with respect to SEQ ID NO: 2. In some embodiments, the αGS may comprise the amino acid sequence of SEQ ID NO: 3, or the amino acid sequence of amino acids 258 to 548 of SEQ ID NO: 3.

In some embodiments, the αGS comprises the amino acid sequence of SEQ ID NO: 3, optionally with from 1 to 20 or from 1 to 10, or from 1 to 5, or from 1 to 3 amino acid modifications independently selected from substitutions, deletions, and insertions, and which improve α-Guaiene titer or percent α-Guaiene with respect to SEQ ID NO: 3. In some embodiments, the αGS comprises from 2 to 20 or from 2 to 10 amino acid substitutions, or from 2 to 5 amino acid substitutions with respect to SEQ ID NO: 3 within positions 258 to 548, and which improve α-Guaiene titer or percent α-Guaiene with respect to the enzyme of SEQ ID NO: 3. For example, the αGS may comprise one or more amino acid substitutions with respect to SEQ ID NO: 3 listed in Table 2, and which improve α-Guaiene titer or percent α-Guaiene with respect to the enzyme of SEQ ID NO: 3.

For example, the αGS in some embodiments comprises the substitution T290A and/or I293F with respect to SEQ ID NO: 3. In some embodiments, the αGS comprises the amino acid sequence of SEQ ID NO: 4. In particular, the substitution I293F may favor INT5 and/or INT6, versus INT4, thereby shifting the product profile toward α-Guaiene and away from α-Bulnesene.

In some embodiments, the αGS comprises the amino acid sequence of SEQ ID NO: 4, optionally with from 1 to 20 or from 1 to 10, or from 1 to 5, or from 1 to 3 amino acid modifications independently selected from substitutions, deletions, and insertions, and which improve α-Guaiene titer or percent α-Guaiene with respect to the enzyme of SEQ ID NO: 4. In some embodiments, the αGS comprises from 2 to 20 or from 2 to 10 amino acid substitutions, or from 2 to 5 amino acid substitutions with respect to SEQ ID NO: 4 within positions 258 to 548, and which improve α-Guaiene titer or percent α-Guaiene with respect to the enzyme of SEQ ID NO: 4. For example, the αGS may comprise one or more amino acid substitutions with respect to SEQ ID NO: 4 at positions selected from 447, 372, 296, 400, 293, 439, 452, 292, 480, 203, 369, and 325 with respect to SEQ ID NO: 4.

In some embodiments, the αGS comprises one or more amino acid modifications with respect to SEQ ID NO: 4 that are selected from Table 3, and which improve α-Guaiene titer or percent α-Guaiene with respect to the enzyme of SEQ ID NO: 4. In some embodiments, the αGS comprises the substitutions L447V, 1400V, and M273I, with respect to SEQ ID NO: 4. For example, the αGS may comprise the amino acid sequence of SEQ ID NO: 5, or the amino acid sequence of amino acids 258 to 548 of SEQ ID NO: 5.

In some embodiments, the αGS comprises the amino acid sequence of SEQ ID NO: 5, optionally with from 1 to 20 or from 1 to 10, or from 1 to 5, or from 1 to 3 amino acid modifications independently selected from substitutions, deletions, and insertions, and which improve α-Guaiene titer or percent α-Guaiene with respect to the enzyme of SEQ ID NO: 5. In some embodiments, the αGS comprises from 2 to 20 or from 2 to 10 amino acid substitutions, or from 2 to 5 amino acid substitutions with respect to SEQ ID NO: 5 within positions 258 to 548, and which improve α-Guaiene titer or percent α-Guaiene with respect to the enzyme of SEQ ID NO: 5. For example, the αGS may comprise one or more amino acid substitutions with respect to SEQ ID NO: 5 as listed in Table 4, and which improve α-Guaiene titer or percent α-Guaiene with respect to the enzyme of SEQ ID NO: 5. In some embodiments, the αGS comprises the substitutions T296V and E325T, with respect to SEQ ID NO: 5. For example, the αGS may comprise the amino acid sequence of SEQ ID NO: 28 or the amino acid sequence of amino acids 258 to 548 of SEQ ID NO: 28.

Thus, the αGS may comprise amino acid substitutions at one or more positions selected from 273, 290, 293, 296, 325, 375, 400, 407, 443, and 447, with respect to SEQ ID NO: 1. For example, the αGS may comprise one or more (e.g., at least 2, 3, 4, 5, 6, 7, 8, 9, or 10) amino acid substitutions selected from M273I, N290T, N290A, 1293F, T296V, E325T, S375A, 1400L, 1400V, F407L, Y443L, Y443V, Y443F, and L447V with respect to SEQ ID NO: 1. In some embodiments, the αGS comprises the amino acid sequence of SEQ ID NO: 28, or comprises the amino acid sequence of residues 258 to 548 of SEQ ID NO: 28.

Accordingly, in one aspect of this disclosure, the invention provides engineered αGS enzymes (and encoding polynucleotides and host cells comprising the same). The αGS enzymes are engineered for productivity and/or improved product profile toward α-Guaiene, and away from the major side product α-Bulnesene. In various embodiments, the αGS enzyme comprises an amino acid sequence that has at least about 90% sequence identity, or at least about 95% sequence identity, or at least about 97% sequence identity, or at least 98% sequence identity, or at least 99% sequence identity with amino acids 258 to 548 of SEQ ID NO: 28, wherein the α-Guaiene Synthase comprises (i.e., retains) a Phenylalanine at the position corresponding to position 293 of SEQ ID NO: 28, and optionally retains a non-aromatic residue (e.g., a residue other than Phenylalanine) at the position corresponding to position 407 of SEQ ID NO: 28. In various embodiments, the αGS comprises one or more of (or two or more, or three of more, or four or more, or five or more, or each of):
an Ile, Leu, or Val at the position corresponding to position 273 of SEQ ID NO: 28;
an Ala, Gly, Thr, or Ser at the position corresponding to position 290 of SEQ ID NO: 28;
a Val, Leu, Ile, or Ala at the position corresponding to position 296 of SEQ ID NO: 28;
a Thr or Ser at the position corresponding to position 325 of SEQ ID NO: 28;
an Ala, Gly, or Leu at the position corresponding to position 375 of SEQ ID NO: 28;
a Val or Leu at the position corresponding to position 400 of SEQ ID NO: 28;
a Leu, Val, or Ile at the position corresponding to position 407 of SEQ ID NO: 28;
a Leu, Val, or Ile at the position corresponding to position 443 of SEQ ID NO: 28; and
a Val at the position corresponding to position 447 of SEQ ID NO: 28.

In various embodiments, the αGS comprises a phenylalanine at the position corresponding to position 293 of SEQ ID NO: 28, and a Leucine at position 407 of SEQ ID NO: 28. Corresponding modifications can be made to other α-Guaiene Synthase enzymes to improve biosynthesis of α-Guaiene, including the αGS enzymes described in WO 2020/051488, which is hereby incorporated by reference in its entirety. Exemplary such mutations are exemplified in Table 14.

In some embodiments, the αGS comprises the amino acid sequence of SEQ ID NO: 28, optionally with from 1 to 20 or from 1 to 10, or from 1 to 5, or from 1 to 3 amino acid modifications independently selected from substitutions, deletions, and insertions. In some embodiments, the αGS comprises from 2 to 20 or from 2 to 10, or from 2 to 5 amino acid modifications with respect to SEQ ID NO: 28 within positions 258 to 548 of SEQ ID NO: 28. In some embodiments, the αGS comprises one or more amino acid modifications listed in Table 5 with respect to SEQ ID NO: 28.

In some embodiments, the αGS comprises at least one of the modifications with respect to SEQ ID NO: 28 selected from G269S, Y21F, Q448V, and A545P. In some embodiments, the αGS comprises at least two of the modifications with respect to SEQ ID NO: 28 selected from G269S, Y21F, Q448V, and A545P. In some embodiments, the αGS comprises the following modifications with respect to SEQ ID NO: 28: G269S, Y21F, Q448V, and A545P. In some embodiments, the αGS comprises the amino acid sequence of SEQ ID NO: 31, or comprises the amino acid sequence of amino acids 258 to 548 of SEQ ID NO: 31.

In some embodiments, the αGS comprises amino acid substitutions at one or more positions selected from 21, 269, 273, 290, 293, 296, 325, 375, 400, 407, 443, 447, 448 and 545 with respect to SEQ ID NO: 1. In some embodiments, the αGS comprises one or more amino acid substitutions selected from Y21F, G269S, M273I, N290T, N290A, 1293F, T296V, E325T, S375A, 1400L, 1400V, F407L, Y443L, Y443V, Y443F, L447V, Q448V, and A545P with respect to SEQ ID NO: 1.

In some embodiments, the αGS comprises the amino acid sequence of SEQ ID NO: 31, optionally with from 1 to 20 or from 1 to 10, or from 1 to 5, or from 1 to 3 amino acid modifications independently selected from substitutions, deletions, and insertions. In some embodiments, the αGS comprises from 2 to 20 or from 2 to 10, or from 2 to 5 amino acid modifications with respect to SEQ ID NO: 31 within positions 258 to 548 of SEQ ID NO: 31. In some embodiments, the one or more amino acid modifications is selected from those listed in Table 6 with respect to SEQ ID NO: 31.

In some embodiments, the αGS comprises at least the modifications V448Q and/or I487D with respect to SEQ ID NO: 31. In some embodiments, the αGS comprises the amino acid sequence of SEQ ID NO: 32, or comprises the amino acid sequence of amino acids 258 to 548 of SEQ ID NO: 32.

In some embodiments, the αGS comprises amino acid substitutions at one or more positions selected from 21, 269, 273, 290, 293, 296, 325, 375, 400, 407, 443, 447, 448, 487, and 545 with respect to SEQ ID NO: 1. In some embodiments, the αGS comprises one or more amino acid substitutions selected from Y21F, G269S, M273I, N290T, N290A, I293F, T296V, E325T, S375A, 1400L, I400V, F407L, Y443L, Y443V, Y443F, L447V, Q448V, I487D, and A545P with respect to SEQ ID NO: 1

In various embodiments, the synthase is recombinantly expressed as known in the art or as described herein. The synthase is optionally purified. In still other embodiments, the synthase is expressed in a host cell that produces farnesyl diphosphate, as described herein.

In some embodiments, the α-Guaiene produced in the αGS reaction is oxidized to rotundone, which can employ an αGO enzyme. In some embodiments, the αGO oxidizes at least one portion of the α-Guaiene to a ketone. In some embodiments, the oxidation of α-Guaiene by αGO results in the production of one or more alcohol intermediates. In some embodiments, the alcohol intermediates are converted to rotundone by one or more alcohol dehydrogenases.

In some embodiments, the αGO enzyme is a cytochrome P450 (CYP450) enzyme. CYP450 enzymes are involved in the formation (synthesis) and breakdown (metabolism) of various molecules and chemicals within cells. CYP450 enzymes have been identified in all kingdoms of life (i.e., animals, plants, fungi, protists, bacteria, archaea, and even in viruses). Illustrative structure and function of CYP450 enzymes are described in Uracher et al., TRENDS in Biotechnology, 24(7): 324-330 (2006).

In some embodiments, the αGO engineered as described herein produces predominantly rotundone and/or rotundol (e.g., greater than 50%) as the oxygenated product from α-Guaiene substrate. In some embodiments, the αGO produces greater than about 75%, or greater than about 80%, or greater than about 85%, or greater than about 90% rotundone and/or rotundol as the oxygenated product from α-Guaiene substrate. Enzyme specificity can be determined in host microbial cells producing α-Guaiene, followed by chemical analysis of total terpenoid products.

In various embodiments, the αGO comprises an amino acid sequence having at least 80% sequence identity to the amino acid sequence of SEQ ID NO: 6. In various embodiments, the αGO comprises an amino acid sequence having at least 85% sequence identity, or at least 90% sequence identity, or at least 95% sequence identity, or at least 97% sequence identity to the amino acid sequence of SEQ ID NO: 6. In various embodiments, the GO comprises from 1 to 20, or from 1 to 10, or from 1 to 5 amino acid modifications with respect to SEQ ID NO: 6. The amino acid modifications can be independently selected from amino acid substitutions, deletion, and insertions, and improve titer and/or profile of rotundone or rotundol as compared to the enzyme defined by SEQ ID NO: 6.

In some embodiments, modifications to enzymes can be informed by construction of a homology model. In some embodiments, selection and modification of enzymes is informed by assaying activity on α-Guaiene substrate. In some embodiments, the amino acid modifications can be selected to improve one or more of: enzyme productivity, selectivity for the desired substrate and/or product, stability, temperature tolerance, and expression in microbial host cells. In accordance with embodiments of this disclosure, the second position of the enzymes described herein can be Ala, which provides for increased stability in microbial cells such as E. coli.

In various embodiments, the αGO comprises a substitution at one or more positions relative to SEQ ID NO: 6 selected from: 497, 235, 451, 72, 490, 496, 368, 318, 387, and 386. In some embodiments, the αGO comprises one or more (e.g., 2, 3, 4, or 5) substitutions selected from Table 6, and which improve production of rotundol or rotundone from α-Guaiene. Such amino acid modifications can improve titer and/or product profile for production of rotundol or rotundone. For example, the αGO may comprise the amino acid substitution M235R and/or E318L with respect to SEQ ID NO: 6. In some embodiments, the αGO comprises the amino acid sequence of SEQ ID NO: 7.

In some embodiments, the αGO comprises a substitution at one or more positions or substitutions from Table 7 relative to SEQ ID NO: 7, and which improve the production of rotundol and/or rotundone relative to the enzyme of SEQ ID NO: 7. For example, the αGO may comprise from 1 to 10 or from 1 to 5 amino acid modifications (independently selected from substitutions, deletions, and insertions) with respect to the enzyme of SEQ ID NO: 7, and which improve the production of rotundol and/or rotundone from α-Guaiene, relative to the enzyme of SEQ ID NO: 7. Such amino acid modifications may be selected from Table 7. For example, the αGO may comprise amino acid substitution selected from I238A and/or S320T with respect to SEQ ID NO: 7. In some embodiments, the αGO comprises the amino acid sequence of SEQ ID NO: 8.

In some embodiments, the αGO comprises the amino acid sequence of SEQ ID NO: 8, optionally having from 1 to 10 or from 1 to 5 amino acid modifications independently selected from amino acid substitutions, insertions, and deletions that further provide improvements in rotundol or rotundone titers or product profile, and/or which improve temperature tolerance or expression or stability in microbial cells. In some embodiments, the αGO comprises one or more amino acid modifications (independently selected from amino acid substitutions, deletions, and insertions) that improve production of rotundol and/or rotundone from α-Guaiene, and which may include one or more (e.g., 2, 3, 4, or 5) amino acid modifications listed in Table 8. In some embodiments, the αGO comprises the substitutions L318A, T320S, and 1490G, with respect to the enzyme of SEQ ID NO. 8. In some embodiments, the αGO comprises the amino acid sequence of SEQ ID NO: 9.

In some embodiments, the αGO comprises the amino acid sequence of SEQ ID NO: 9, optionally having from 1 to 10 or from 1 to 5 amino acid modifications independently selected from amino acid substitutions, insertions, and deletions that further provide improvements in rotundol or rotundone titers or product profile, and/or which improve temperature tolerance or expression or stability in microbial cells. In some embodiments, the αGO comprises one or more amino acid modifications (independently selected from amino acid substitutions, deletions, and insertions) that improve production of rotundol and/or rotundone from α-Guaiene, and which may include one or more (e.g., 2, 3, 4, or 5) amino acid modifications listed in Table 9, relative to SEQ ID NO: 9. In some embodiments, the αGO comprises substitution(s) selected from T489Q and H495S, with respect to the enzyme of SEQ ID NO. 9. In some embodiments, the αGO comprises the amino acid sequence of SEQ ID NO: 29.

In some embodiments, the αGO comprises the amino acid sequence of SEQ ID NO: 29, optionally having from 1 to 10 or from 1 to 5 amino acid modifications independently selected from amino acid substitutions, insertions, and deletions that further provide improvements in rotundol or rotundone titers or product profile, and/or which improve temperature tolerance or expression or stability in microbial cells. In some embodiments, the αGO comprises one or more amino acid modifications (independently selected from amino acid substitutions, deletions, and insertions) that improve production of rotundol and/or rotundone from α-Guaiene, and which may include one or more (e.g., 2, 3, 4, or 5) amino acid modifications listed in Table 10, relative to SEQ ID NO: 29. In some embodiments, the αGO comprises the substitution D440G, with respect to the enzyme of SEQ ID NO. 29. In some embodiments, the αGO comprises the amino acid sequence of SEQ ID NO: 30.

In some embodiments, the αGO comprises the amino acid sequence of SEQ ID NO: 30, optionally having from 1 to 10 or from 1 to 5 amino acid modifications independently selected from substitutions, insertions, and deletions. In some embodiments, the αGO comprises one or more amino acid modifications with respect to SEQ ID NO: 30 that are selected from Table 11.

In some embodiments, the αGO comprises at least one substitution selected from E184A, H389Y and R501H with respect to SEQ ID NO: 30. In some embodiments, the αGO comprises at least two substitutions selected from E184A, H389Y and R501H with respect to SEQ ID NO: 30. In some embodiments, the αGO comprises E184A, H389Y and R501H substitutions with respect to SEQ ID NO: 30. In some embodiments, the αGO comprises the amino acid sequence of SEQ ID NO: 33, or an amino acid sequence having at least 80%, or at least 85%, at least 90%, or at least 95%, at least 97% sequence identity thereto.

In some embodiments, the αGO comprises the amino acid sequence of SEQ ID NO: 33, optionally having from 1 to 10 or from 1 to 5 amino acid modifications independently selected from substitutions, insertions, and deletions. In some embodiments, the αGO comprises one or more amino acid modifications with respect to SEQ ID NO: 33 that are selected from Table 11.

Accordingly, one aspect of the disclosure provides engineered αGO enzymes (and encoding polynucleotides and host cells comprising the same). The αGO enzyme is engineered for productivity and/or improved product profile toward rotundol or rotundone. In some embodiments, the αGO enzyme comprises an amino acid sequence that has at least about 90%, at least about 95%, or at least about 97% sequence identity to SEQ ID NO: 30, wherein the αGO comprises at least two, three, four, or five (or each) of:
a Ala or Gly at the position corresponding to position 184 of SEQ ID NO: 30;
an Arg, Lys, Ser, or Thr at the position corresponding to position 235 of SEQ ID NO: 30;
an Ala, Leu, Thr, or Gly at the position corresponding to position 238 of SEQ ID NO: 30;
a Ala or Gly at the position corresponding to position 318 of SEQ ID NO: 30;
a Phe, Tyr, Trp at the position corresponding to position 389 of SEQ ID NO: 30;
a Gly, Ala, or Ser at the position corresponding to position 490 of SEQ ID NO: 30;
a Gln, Lys, Asn, Met, Ser, Glu at the position corresponding to position 489 of SEQ ID NO: 30;
a Ser, Asn, or Thr at the position corresponding to position 495 of SEQ ID NO: 30;
a Gly, Ala, or Asn at the position corresponding to position 440 of SEQ ID NO: 30; and
a His at the position corresponding to position 501 of SEQ ID NO: 30.

In some embodiments, the αGO enzyme is co-expressed in a host cell producing α-Guaiene, such as a host cell described herein (including a host cell co-expressing an engineered αGS described herein). In some embodiments, the oxidase is co-expressed in a host cell with a heterologous cytochrome P450 reductase or alcohol dehydrogenase as described below.

In some embodiments, the αGO enzyme is engineered to have a deletion of all or part of the wild type N-terminal transmembrane region, with the addition of a transmembrane domain derived from a microbial (e.g., E. coli) inner membrane cytoplasmic C-terminus protein. In various embodiments, the transmembrane domain is a single-pass transmembrane domain. In various embodiments, the transmembrane domain (or “N-terminal anchor”) is derived from an E. coli gene selected from waaA, ypfN, yhcB, yhbM, yhhm, zipA, ycgG, dj1A, sohB, 1pxK, F11O, motA, htpx, pgaC, ygdD, hemr, and ycls. These genes were identified as inner membrane cytoplasmic C-terminus proteins through bioinformatic prediction as well as experimental validation. See U.S. Pat. No. 10,774,314, which is hereby incorporated by reference in its entirety. In some embodiments, when considering percent identity between αGO enzymes, the E. coli N-terminal transmembrane region is not included in such determinations.

In some embodiments, the αGO is expressed in a cell does that does not express an αGS, allowing for enzymatic biotransformation of α-Guaiene fed to the cells, which can take place with whole cells or whole or partially purified extracts of the cells.

In still other embodiments, the αGO (optionally with an ADH) is provided in a purified recombinant form for production of rotundone from α-Guaiene, or (2R)-rotundol or (2S)-rotundol, in a cell free system.

In some embodiments, the αGO enzyme requires the presence of an electron transfer protein capable of transferring electrons to the enzyme. In some embodiments, this electron transfer protein is a cytochrome P450 reductase (CPR), which can be co-expressed with the αGO in the microbial host cell. Exemplary P450 reductase enzymes include those shown herein as SEQ ID NOs: 20 to 27, or a variant thereof. For example, the cytochrome P450 reductase may comprise an amino acid sequence that is at least about 70%, or at least about 80%, or at least about 85%, or at least about 90%, or at least about 95%, or at least about 97% identical to one of SEQ ID NOS: 20 to 27. In some embodiments, the P450 reductase comprises an amino acid sequence having at least about 70%, or at least about 80%, or at least about 85%, or at least about 90%, or at least about 95%, or at least about 97% (or 100%) sequence identity to SEQ ID NO: 20. In some embodiments, the P450 reductase comprises an amino acid sequence having at least about 70%, or at least about 80%, or at least about 85%, or at least about 90%, or at least about 95%, or at least about 97% (or 100%) sequence identity to SEQ ID NO: 34. In some embodiments, the P450 reductase comprises an amino acid sequence having at least about 70%, or at least about 80%, or at least about 85%, or at least about 90%, or at least about 95%, or at least about 97% (or 100%) sequence identity to SEQ ID NO: 35. In some embodiments, the P450 reductase comprises an amino acid sequence having at least about 70%, or at least about 80%, or at least about 85%, or at least about 90%, or at least about 95%, or at least about 97% (or 100%) sequence identity to SEQ ID NO: 21. In some embodiments, the P450 reductase comprises an amino acid sequence having at least about 70%, or at least about 80%, or at least about 85%, or at least about 90%, or at least about 95%, or at least about 97% (or 100%) sequence identity to SEQ ID NO: 36.

In some embodiments, the αGO reaction results in hydroxylation of α-Guaiene, thereby producing one or more alcohol intermediates, e.g., (2R)-rotundol or (2S)-rotundol (see FIG. 1B). In some embodiments, the αGO further oxidizes at least a portion of the α-Guaiene to a ketone. In some embodiments, the alcohol intermediates (e.g., (2R)-rotundol or (2S)-rotundol) are converted to rotundone by one or more alcohol dehydrogenases (ADHs). Thus, in some embodiments, the microbial host cell expresses one or more alcohol dehydrogenases (ADH). In various embodiments, the heterologous biosynthesis pathway further comprises an alcohol dehydrogenase. Exemplary alcohol dehydrogenase enzymes are provided herein as SEQ ID NOS: 10 to 19. In various embodiments, the alcohol dehydrogenase comprises an amino acid sequence that has at least about 70%, or at least about 80%, or at least about 85%, or at least about 90%, or at least about 95%, or at least about 97% sequence identity to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19.

In some embodiments, the amino acid modifications to the ADH can be selected to improve one or more of: enzyme productivity, selectivity for the desired substrate and/or product, stability, temperature tolerance, and expression in microbial host cells. In various embodiments, the alcohol dehydrogenase comprises an amino acid sequence that is at least about 70%, or at least about 80%, or at least about 85%, or at least about 90%, or at least about 95%, or at least about 97% sequence identity to SEQ ID NO: 10. In various embodiments, the alcohol dehydrogenase comprises an amino acid sequence that is at least about 70%, or at least about 80%, or at least about 85%, or at least about 90%, or at least about 95%, or at least about 97% sequence identity to SEQ ID NO: 14. In various embodiments, the alcohol dehydrogenase comprises an amino acid sequence that is at least about 70%, or at least about 80%, or at least about 85%, or at least about 90%, or at least about 95%, or at least about 97% sequence identity to SEQ ID NO: 19. In various embodiments, the alcohol dehydrogenase comprises an amino acid sequence that is at least about 70%, or at least about 80%, or at least about 85%, or at least about 90%, or at least about 95%, or at least about 97% sequence identity to SEQ ID NO: 18. In various embodiments, the alcohol dehydrogenase comprises an amino acid sequence that is at least about 70%, or at least about 80%, or at least about 85%, or at least about 90%, or at least about 95%, or at least about 97% sequence identity to SEQ ID NO: 11. In various embodiments, the alcohol dehydrogenase comprises an amino acid sequence that is at least about 70%, or at least about 80%, or at least about 85%, or at least about 90%, or at least about 95%, or at least about 97% sequence identity to SEQ ID NO: 17. In various embodiments, the alcohol dehydrogenase comprises an amino acid sequence that is at least about 70%, or at least about 80%, or at least about 85%, or at least about 90%, or at least about 95%, or at least about 97% sequence identity to SEQ ID NO: 15.

Conversion of IPP and DMAPP precursors to farnesyl diphosphate (FPP) in host cells is typically through the action of a farnesyl diphosphate synthase (FPPS). Exemplary FPPS enzymes are disclosed in US 2018/0135081, which is hereby incorporated by reference in its entirety. In various embodiments, the host cell is a microbial host cell overexpressing one or more enzymes in the methylerythritol phosphate (MEP) or the mevalonic acid (MVA) pathway.

In various embodiments, one or more heterologous enzymes of the biosynthesis pathway are expressed from extrachromosomal elements (such as plasmids or bacterial artificial chromosomes), and/or are expressed from genes that are chromosomally integrated. In various embodiments, the αGS and αGO (optionally with an FPPS, cytochrome P450 reductase, and/or ADH) are expressed together in an operon, or are expressed individually.

In some embodiments, the microbial host cell is also engineered to express or overexpress one or more enzymes in the methylerythritol phosphate (MEP) and/or the mevalonic acid (MVA) pathway to catalyze isopentenyl pyrophosphate (IPP) and dimethylallyl pyrophosphate (DMAPP) from glucose or other carbon source.

In some embodiments, the microbial host cell is engineered to express or overexpress one or more enzymes of the MEP pathway. In some embodiments, the MEP pathway is increased and balanced with downstream pathways by providing duplicate copies of certain rate-limiting enzymes. The MEP (2-C-methyl-D-erythritol 4-phosphate) pathway, also called the MEP/DOXP (2-C-methyl-D-erythritol 4-phosphate/l-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the microbial host cell expresses or overexpresses of one or more of dxs, ispC, ispD, ispE, ispF, ispG, ispH, idi, ispA, or modified variants thereof, which results in the increased production of IPP and DMAPP. In some embodiments, rotundone is produced at least in part by metabolic flux through an MEP pathway, and wherein the microbial host cell has at least one additional gene copy of one or more of dxs, ispC, ispD, ispE, ispF, ispG, ispH, idi, ispA, or modified variants thereof.

In some embodiments, the microbial host cell is engineered to express or overexpress one or more enzymes of the MVA pathway. The MVA pathway refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-COA synthase (HMGS)); (c) converting HMG-COA to mevalonate (e.g., by action of HMG-COA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MVA pathway, are described in U.S. Pat. No. 7,667,017, which is hereby incorporated by reference in its entirety. In some embodiments, the microbial host cell expresses or overexpresses one or more of acetoacetyl-CoA thiolase, HMGS, HMGR, MK, PMK, and MPD or modified variants thereof, which results in the increased production of IPP and DMAPP. In some embodiments, rotundone is produced at least in part by metabolic flux through an MVA pathway, and wherein the microbial host cell has at least one additional gene copy of one or more of acetoacetyl-CoA thiolase, HMGS, HMGR, MK, PMK, MPD, or modified variants thereof.

In some embodiments, the microbial host cell is engineered to increase production of IPP and DMAPP from glucose as described in U.S. Pat. Nos. 10,662,442 and 10,480,015, the contents of which are hereby incorporated by reference in their entireties. For example, in some embodiments the microbial host cell overexpresses MEP pathway enzymes, with balanced expression to push/pull carbon flux to IPP and DMAPP. In some embodiments, the microbial host cell is engineered to increase the activity of Fe—S cluster proteins (including by heterologous expression of one or more oxidoreductases), so as to support higher activity of IspG and IspH, which are Fe—S enzymes. In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux. In some embodiments, the host cell is engineered to downregulate the ubiquinone biosynthesis pathway, e.g., by reducing the expression or activity of IspB, which uses IPP and FPP substrate.

In still other embodiments, microbial cells expressing FPPS, αGS, and αGO co-express an isoprenol utilization pathway as described in US 2019/0367950, which is hereby incorporated by reference in its entirety. Such cells can produce IPP and DMAPP precursors from prenol and/or isoprenol substrate provided to the culture.

In some embodiments, the microbial host cell is a bacterium selected from Escherichia spp., Bacillus spp., Corynebacterium spp., Rhodobacter spp., Zymomonas spp., Vibrio spp., and Pseudomonas spp. For example, in some embodiments, the bacterial host cell is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterial host cell is E. coli.

In some embodiments, the microbial host cell is a species of Saccharomyces, Pichia, or Yarrowia, including, but not limited to, Saccharomyces cerevisiae, Pichia pastoris, and Yarrowia lipolytica.

Manipulation of the expression of genes and/or proteins, including gene modules, can be achieved through various methods. For example, expression of genes or operons can be regulated through selection of promoters, such as inducible or constitutive promoters, with different strengths (e.g., strong, intermediate, or weak). Several non-limiting examples of promoters of different strengths include Trc, T5 and T7. Additionally, expression of genes or operons can be regulated through manipulation of the copy number of gene or operon in the cell. In some embodiments, expression of genes or operons can be regulated through manipulating the order of the genes within a module, where the genes transcribed first are generally expressed at a higher level. In some embodiments, expression of genes or operons is regulated through integration of one or more genes or operons into the chromosome.

Optimization of protein expression can also be achieved through selection of appropriate promoters and ribosomal binding sites. In some embodiments, this may include the selection of high-copy number plasmids, or single-, low- or medium-copy number plasmids. The step of transcription termination can also be targeted for regulation of gene expression, through the introduction or elimination of structures such as stem-loops.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA. The heterologous DNA is placed under operable control of transcriptional elements to permit the expression of the heterologous DNA in the host cell.

In some embodiments, endogenous genes of the microbial host cell are edited. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

In another aspect, the present disclosure provides a method for making rotundone. The method comprises providing a microbial host cell as disclosed herein. The microbial host cell expresses an αGS and/or an αGO enzyme, as described herein. Cells expressing an αGO enzyme can be used for bioconversion of α-Guaiene using whole cells or cell extracts. Cells expressing an αGO enzyme and an αGS enzyme can produce rotundone from any suitable carbon source. In some embodiments, the microbial host cell further expresses one or more alcohol dehydrogenases (ADHs), such as those disclosed herein. Cells expressing ADHs can convert alcohol intermediates produced by the αGO reaction into rotundone.

In some embodiments, microbial host cells expressing an αGS and an αGO is cultured to produce rotundone. The microbial cells can be cultured with carbon substrates (sources) such as C1, C2, C3, C4, C5, and/or C6 carbon substrates. In exemplary embodiments, the carbon source(s) can be selected from glucose, sucrose, fructose, xylose, and/or glycerol. Culture conditions are generally selected from aerobic, microaerobic, and anerobic.

In various embodiments, the microbial host cell is cultured at a temperature between 22° C. and 37° C. While commercial biosynthesis in bacteria such as E. coli can be limited by the temperature at which overexpressed and/or foreign enzymes (e.g., enzymes derived from plants) are stable, recombinant enzymes (including the terpenoid synthase) may be engineered to allow for cultures to be maintained at higher temperatures, resulting in higher yields and higher overall productivity. In some embodiments, the host cell is a bacterial host cell, and culturing is conducted at about 22° C. or greater, about 23° C. or greater, about 24° C. or greater, about 25° C. or greater, about 26° C. or greater, about 27° C. or greater, about 28° C. or greater, about 29° C. or greater, about 30° C. or greater, about 31° C. or greater, about 32° C. or greater, about 33° C. or greater, about 34° C. or greater, about 35° C. or greater, about 36° C. or greater, or about 37° C.

Rotundone can be extracted from media and/or whole cells, and the rotundone recovered. In some embodiments, the oxygenated rotundone product is recovered and optionally enriched by fractionation (e.g. fractional distillation). The oxygenated product can be recovered by any suitable process, including partitioning the desired product into an organic phase. The production of the desired product can be determined and/or quantified, for example, by gas chromatography (e.g., GC-MS). The desired product can be produced in batch or continuous bioreactor systems. Production of product, recovery, and/or analysis of the product can be done as described in US 2012/0246767, U.S. Pat. Nos. 10,501,760, 10,934,564, which are hereby incorporated by reference in its entirety. For example, in some embodiments, oxidized oil is extracted from aqueous reaction medium, which may be done by partitioning into an organic phase, followed by fractional distillation. Sesquiterpene and sesquiterpenoid components of fractions may be measured quantitatively by GC/MS, followed by blending of the fractions.

In some embodiments, the microbial host cells and methods disclosed herein are suitable for commercial production of rotundone, that is, the microbial host cells and methods are productive at commercial scale. In some embodiments, the size of the culture is at least about 100 L, at least about 200 L, at least about 500 L, at least about 1,000 L, at least about 10,000 L, at least about 100,000 L, or at least about 1,000,000 L. In some embodiment, the culturing may be conducted in batch culture, continuous culture, or semi-continuous culture.

In some aspects, the present disclosure provides methods for making a product comprising rotundone, including flavor and fragrance compositions or products. In some embodiments, the method comprises producing rotundone as described herein through microbial culture, recovering the rotundone, and incorporating the rotundone into the flavor or fragrance composition, or a consumable product (e.g., a food product).

As exemplified and demonstrated herein with regard to the engineering of αGS, in another aspect the invention provides methods for engineering terpene synthase enzymes (and methods of using the same) by favoring certain carbocation catalytic intermediates over others. For example, the method comprises providing a terpene synthase amino acid sequence (e.g., a Class I Terpene synthase amino acid sequence), where the terpene synthase is capable of catalyzing cyclization of a prenyl diphosphate (such as geranyl diphosphate, geranylgeranyl diphosphate, or farnesyl diphosphate) to produce a target cyclic terpenoid and one or more non-target cyclic terpenoids through deprotonation of a series of cyclic carbocation intermediates. As used herein, the term “target cyclic terpenoid” refers to the desired product of the terpene synthase reaction, and generally will be the predominant product when using the engineering techniques described herein. As used herein, the “non-target cyclic terpenoid(s)” refer to side products of the same reaction (between the prenyl diphosphate and the terpene synthase enzyme). In various embodiments, synthesis of the target cyclic terpenoid versus non-target cyclic terpenoids will be based on the position of deprotonation of carbocation intermediates. In various embodiments, the terpene synthase reaction with a prenyl diphosphate substrate involves at least two, or at least three, or at least four potential catalytic intermediates having different positions for a carbocation, deprotonation of which controls formation of a target or non-target terpenoid. In various embodiments, the target cyclic terpenoid is a sesquiterpenoid, a triterpenoid, a diterpenoid, or a monoterpenoid. The target cyclic terpenoid can be monocylic, bicyclic, or tricyclic, in various embodiments.

The terpene synthase amino acid sequence will comprise one or more amino acid modifications (with respect to a wild type or parent terpene synthase enzyme) so as: to position an aromatic side chain to stabilize a carbocation catalytic intermediate that deprotonates to the target cyclic terpenoid; and/or to remove or shift one or more aromatic or aliphatic side chains to destabilize a carbocation intermediate that deprotonates to at least one non-target cyclic terpenoid. These modifications alter the product profile toward the target terpenoid, and away from the non-target terpenoid. The engineered terpene synthase enzyme may be recombinantly produced, and the synthase may be expressed in microbial cells for microbial production of the desired compound as described herein.

In various embodiments, the amino acid modifications to the terpene synthase are guided by a structural model of the terpene synthase. In some embodiments, the structural model is a homology model. An exemplary homology model can be based on structural coordinates for 5-epi-aristolochene synthase. See, U.S. Pat. Nos. 6,645,762, 6,495,354, and 6,645,762, which are hereby incorporated by reference in their entireties.

This aspect of the invention can be used to engineer various terpene synthase enzymes, including but not limited to a guaiene synthase, a valencene synthase, a sabinene synthase, a limonene synthase, a cineole synthase, a cubebol synthase, a kaurene synthase, a humulene synthase, a carene synthase, a terpineol synthase, a thujene synthase, a terpinene synthase, pinene synthase, a germacrene synthase, a patchoulol synthase, a santalene synthase, a sclareol synthase, a cadinene synthase, a cedrol synthase, a bisabolene synthase, a caryophyllene synthase, a longifolene synthase, bisobolol synthase, a copaene synthase, a muuroladiene synthase, a bergamotene synthase, an amorphadiene synthase, taxadiene synthase, a levopimaradiene synthase, an abietadiene synthase, an amyrin synthase, a selinene synthase, an epi-aristocholene synthase, a vetispiradiene synthase, an epicedrol synthase, an elemene synthase, a zingiberene synthase, a lupeol synthase, a dammaranediol synthase, and a cubcurbitadienol synthase, among others.

In some embodiments, amino acid side chains are identified that are within a distance of about 15 Ang, or within a distance of about 12 Ang, or within a distance of about 7 Ang. of the substrate in the active site, or within this distance of a carbocation of a catalytic intermediate that deprotonates to the desired product or a major side product. These residues are evaluated for creating cation-π interactions to stabilize the desired carbocation, for example, by substituting a non-aromatic residue for an aromatic residue (such as phenylalanine), or for shifting/optimizing the position of an existing aromatic residue. In addition, these residues are evaluated for removing cation-π interactions or other interactions that stabilize a carbocation intermediate that deprotonates to a non-target terpenoid.

In various embodiments, an aromatic side chain is added and/or positioned to provide or increase a cation-π interaction; and an aromatic side chain is removed or shifted to destabilize or remove a cation-π interaction. For example, a non-aromatic side chain in a wild-type or parent enzyme can be substituted with an aromatic side chain, wherein the aromatic side chain forms a cation-π interaction with the carbocation that deprotonates to the target cyclic terpenoid. Further, an aromatic side chain in the wild-type or parent enzyme can be substituted with a non-aromatic side chain, wherein the aromatic side chain in the wild-type or parent enzyme forms a cation-π interaction with the carbocation that deprotonates to a non-target cyclic terpenoid.

While embodiments of the invention may employ any amino acid with an aromatic side chain, such as phenylalanine, tyrosine, tryptophan, or histidine, in various embodiments, the aromatic side chain is phenylalanine.

The one or more amino acid modifications to the terpene synthase will position the center of the aromatic group (e.g., the benzyl ring of a phenylalanine side chain) within about 6 or 5 Angstroms of the carbocation that deprotonates to the target cyclic terpenoid. In particular embodiments, the amino acid modifications position the center of an aromatic group (such as the benzyl ring of a phenylalanine side chain) within about 4.5 or within about 4.0 Angstroms of the carbocation that deprotonates to the target cyclic terpenoid. In some embodiments, the amino acid modifications position the center of the aromatic group (such as the benzyl ring of a phenylalanine side chain) from about 3.5 to about 5.0 Angstroms of the carbocation that deprotonates to the target cyclic terpenoid.

In these or other embodiments, the amino acid modifications result in removal or positioning of all aromatic or aliphatic residues to a distance that is at least about 6 Angstroms from the carbocation that deprotonates to the major non-target terpenoid. By positioning aromatic and aliphatic resides away from the carbocation that deprotonates to a non-target terpenoid, this carbocation is disfavored, thereby reducing formation of the non-target terpenoid.

In various embodiments, one or more amino acid modifications are made to secondary structure elements of a Class I Terpene Synthase enzyme selected from the G2 helices, the D helices, the J helices, and the C helices. These structural elements form part of the terpene synthase active site. These structural elements are shown for an αGS in Table 8. For example, a non-aromatic residue in the G2 helices, the D helices, the J helices, or the C helices may be substituted with an aromatic residue, which is optionally phenylalanine, to thereby stabilize the carbocation that protonates to the target cyclic terpenoid. In these or other embodiments, an aromatic or aliphatic residue in the G2 helices, the D helices, the J helices, or the C helices that stabilizes a carbocation that deprotonates to a non-target terpenoid is substituted with a non-aromatic or non-aliphatic residue.

In various embodiments, the terpene synthase is expressed in a host cell that produces the prenyl diphosphate, and optionally one or more oxidase enzymes (including but not limited to cytochrome P450 enzymes and reductase partners) that oxygenate the target cyclic terpenoid.

In various embodiments, the method further comprises recovering the target cyclic terpenoid from the reaction or culture. The methods described herein for culturing microbial cells and recovering rotundone, can be employed for other terpenoid products.

As used in this specification and the appended claims, the singular forms “a”, “an” and “the” include plural referents unless the content clearly dictates otherwise. For example, reference to “a cell” includes a combination of two or more cells, and the like.

As used herein, the term “about” in reference to a number is generally taken to include numbers that fall within a range of 10% in either direction (greater than or less than) of the number.

### EXAMPLES

Rotundone is a bicyclic sesquiterpene and is responsible for pepper aromas in grapes and wine and in herbs and spices, especially black and white pepper, where it has a high odor activity value (OAV). The biosynthesis of rotundone involves enzymatic cyclization of the C15 sesquiterpene precursor substrate farnesyl diphosphate (FPP) to α-Guaiene. In addition to α-Guaiene, this step often results in substantial amount of α-Bulnesene as a major side product, in addition to several minor side products. The products and proposed catalytic intermediates (INT1-INT7) for this cyclization step are illustrated in FIG. 1A.

Enzymatic oxygenation of α-Guaiene produces rotundone, and the reaction may proceed through an alcohol intermediate (FIG. 1B). For example, α-Guaiene may be converted to (2S)-rotundol or (2R)-rotundol by the action of α-Guaiene oxidase (αGO), and the alcohol intermediate (rotundol) can be converted to rotundone by the action of the αGO or an alcohol dehydrogenase.

Rotundone can be produced by biosynthetic fermentation processes, using microbial strains that produce high levels of MEP pathway products, along with heterologous expression of rotundone biosynthesis enzymes, including, enzymes that catalyze: 1) cyclization of FPP to α-Guaiene; 2) oxidation of α-Guaiene to rotundone, and which can optionally include 3) dehydrogenation of rotundol to rotundone. For example, in bacteria such as E. coli, isopentenyl pyrophosphate (IPP) and dimethylallyl pyrophosphate (DMAPP) can be produced from glucose or other carbon sources, and which can be converted to farnesyl diphosphate (FPP) by recombinant farnesyl diphosphate synthase (FPPS). FPP is converted to α-Guaiene by αGS. The α-Guaiene is converted to rotundol or rotundone by oxygenation reaction catalyzed by αGO. In instances where the αGO enzyme catalyzes the production of (2S)-rotundol or (2R)-rotundol from α-Guaiene, the conversion of rotundol to rotundone may be catalyzed by a dehydrogenase.

### Example 1: Engineering of an α-Guaiene Synthase Enzyme

Using an E. coli background strain that produces high levels of the MEP pathway products IPP and DMAPP (see U.S. Pat. Nos. 10,662,442; 10,480,015; and 10,774,346, which are hereby incorporated by reference), a candidate αGS enzyme was engineered for production of improved α-Guaiene titers as well as profiles (i.e., amount of α-Guaiene with respect to side products). Engineered enzymes were screened by co-expression with FPPS in the E. coli cells engineered for high production of MEP pathway products. Fermentation was performed in 96 well plates for 72 hours.

A candidate αGS (Aquilaria crassna DGuaS3) is disclosed in WO 2020/051488, which is hereby incorporated by reference in its entirety, and disclosed herein as SEQ ID NO: 1 (termed “GS0”). A homology model for GS0 was constructed to evaluate reaction chemistry and identify potential amino acid modifications to improve performance. Using this model, mutations were designed using a variety of analyses.

Three amino acid substitutions (S375A, F407L, and Y443L) were identified during Round 1 engineering (see WO 2020/051488, which is hereby incorporated by reference in its entirety). In particular, the substitution F407L may disfavor the stabilization of INT4, and push the enzyme to favor INT5 for higher α-Guaiene production. See FIG. 2. Mutation S375A may disfavor the deprotonation of INT5 to α-Bulnesene, and consequently favor the INT5 to α-Guaiene process. The αGS disclosed as αGS1 (SEQ ID NO: 2) contains these three amino acid substitutions (S375A, F407L, and Y443L) with respect to SEQ ID NO: 1.

96 amino acid substitutions were selected for screening in Round 2. Amino acid substitutions were evaluated for changes to α-Guaiene titer as well as the % α-Guaiene (of total product). The following amino acid substitutions showed beneficial changes, particularly to α-Guaiene titer:

TABLE 1
Round 2 of α-GS Engineering

| | Fold Improvement of | Fold Improvement |
|---|---|---|
| Mutation | α-Guaiene titer | % α-Guaiene of total |
| N290T | 2.18 | 1.10 |
| E325S | 1.75 | 1.06 |
| A499P | 1.53 | 1.04 |
| T495E | 1.46 | 1.07 |
| M341L | 1.46 | 1.05 |
| M273I | 1.41 | 1.11 |
| L447V | 1.31 | 1.05 |
| G294A | 1.28 | 1.07 |
| L439I | 1.12 | 0.94 |
| S504M | 1.11 | 1.00 |
| F369M | 1.09 | 0.99 |
| A206E | 1.07 | 0.92 |

In particular, the substitution N290T was identified as having a significant improvement in titer, as well as some improvement in % α-Guaiene. While several amino acid substitutions in Round 2 showed improvements in titer, changes in % α-Guaiene were less dramatic.

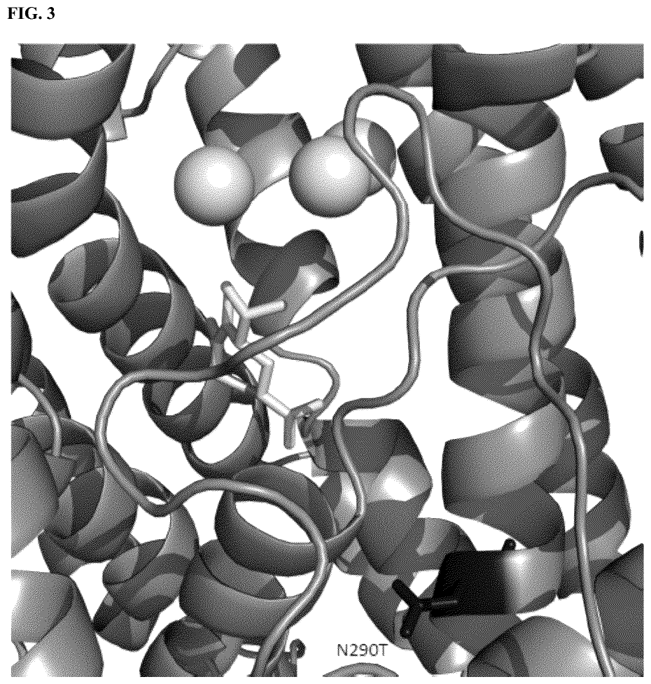

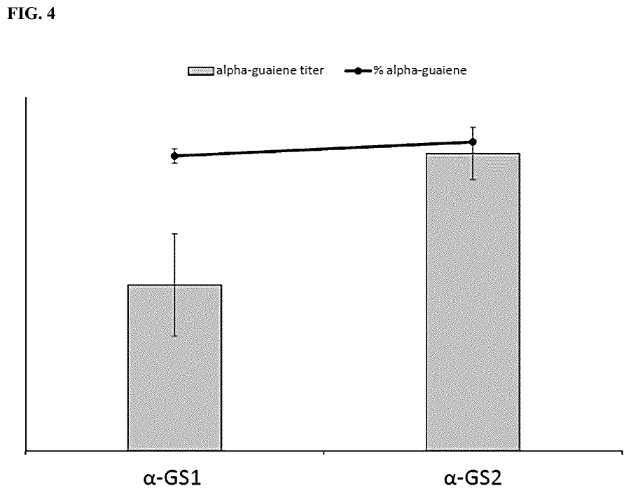

FIG. 4 compares α-Guaiene titer (gray bars) and % α-Guaiene (line) produced by fermentation of E. coli strains expressing αGS1 (after Round 1) (SEQ ID NO: 2) or αGS2 (after Round 2) (SEQ ID NO: 3) in 96 well plates for 72 hours. αGS2 contains the following amino acid substitutions with respect to αGS0: S375A, F407L, and Y443L, and N290T. Compared to α-GS1, α-GS2 provides approximately twice the α-Guaiene titer of α-GS1, with a small improvement in % α-Guaiene.

An additional 348 mutations in αGS2 were screened in Round 3. Amino acid substitutions were evaluated for changes to α-Guaiene titer as well as % α-Guaiene (of total product). The following amino acid substitutions showed significant improvement in one or more of these parameters:

TABLE 2
Round 3 of α-GS Engineering

| | Fold Improvement | Fold Improvement in |
|---|---|---|
| Mutation | in α-Guaiene titers | % α-Guaiene of total |
| A499P | 2.25 | 1.05 |
| V516M | 2.03 | 1.01 |
| F513Y | 1.94 | 1.00 |
| Y527F | 1.92 | 1.10 |
| M481L | 1.90 | 1.02 |
| A462G | 1.74 | 1.04 |
| I444M | 1.70 | 0.99 |
| M273I | 1.65 | 1.09 |
| I440L | 1.64 | 1.02 |
| M520I | 1.63 | 1.00 |
| A267V | 1.61 | 0.97 |
| R468K | 1.57 | 0.84 |
| E518D | 1.55 | 1.01 |
| C189Q | 1.55 | 1.01 |
| F513L | 1.54 | 1.11 |
| V368A | 1.53 | 1.06 |
| S504M | 1.52 | 1.00 |
| L447V | 1.49 | 0.99 |
| I444M | 1.48 | 0.98 |
| V463I | 1.44 | 0.88 |
| S438A | 1.43 | 1.04 |
| I196Q | 1.42 | 0.99 |
| E325S | 1.40 | 1.00 |
| V463L | 1.39 | 0.99 |
| T290A/T296V | 1.38 | 1.01 |
| I440V | 1.38 | 1.01 |
| L439K | 1.37 | 1.01 |
| K173T | 1.37 | 1.00 |
| M406A | 1.36 | 1.03 |
| M341L | 1.35 | 1.04 |
| M274L | 1.34 | 0.99 |
| Q448R | 1.33 | 0.97 |
| E140K | 1.32 | 0.99 |
| Q203P | 1.32 | 0.96 |
| R374K | 1.32 | 0.98 |
| K480E | 1.31 | 0.99 |
| G119Q | 1.31 | 0.98 |
| F513M | 1.30 | 0.99 |
| N201G | 1.30 | 1.01 |
| L443V | 1.30 | 1.02 |
| T290A/G294A | 1.30 | 0.97 |
| C149S | 1.30 | 1.00 |
| K220H | 1.30 | 1.00 |
| P212S | 1.30 | 1.00 |
| I399V | 1.28 | 1.07 |
| I485V | 1.28 | 1.00 |
| I221N | 1.28 | 0.99 |
| T290A/R172T | 1.27 | 1.00 |
| F369M | 1.27 | 0.97 |
| H33Y | 1.26 | 1.00 |
| L439T | 1.24 | 0.96 |
| R374Q | 1.21 | 0.95 |
| I400L | 1.21 | 1.04 |
| K287E | 1.21 | 0.90 |
| I440M | 1.20 | 1.05 |
| Y271F | 1.20 | 0.95 |
| S171T | 1.20 | 1.00 |
| I440A | 1.20 | 0.93 |
| T514V | 1.19 | 0.89 |
| T514S | 1.18 | 0.98 |
| I372Q | 1.18 | 1.18 |
| A206E | 1.18 | 0.99 |
| L443F | 1.17 | 0.97 |
| D500T | 1.16 | 1.01 |
| L443Y | 1.15 | 0.96 |
| M406T | 1.14 | 1.01 |
| T302S | 1.12 | 1.01 |
| S461K | 1.12 | 1.12 |
| L108Y | 1.12 | 1.00 |
| L439V | 1.12 | 0.95 |
| A177S | 1.12 | 1.02 |
| N493I | 1.11 | 0.90 |
| R374G | 1.10 | 0.98 |
| S528G | 1.10 | 0.95 |
| T290A/I292V | 1.10 | 0.91 |
| I372L | 1.10 | 0.90 |
| I408T | 1.08 | 0.96 |
| F513L | 1.06 | 1.06 |
| P23A | 1.06 | 0.98 |
| I444K | 1.05 | 0.82 |
| I400T | 1.05 | 1.03 |
| I400V | 1.05 | 1.03 |
| T290A/I293F | 0.42 | 1.38 |

It was observed that the substitution at position 461 (S461K) positively impacted both α-Guaiene titer and % of total. The dual mutation T290A/I293F showed a substantial impact on % α-Guaiene. The I293F substitution may favor stabilization of INT5 with cation-π interactions and support higher α-Guaiene production. See FIG. 5. The T290A/I293F substitutions were added to αGS2 to create αGS3.

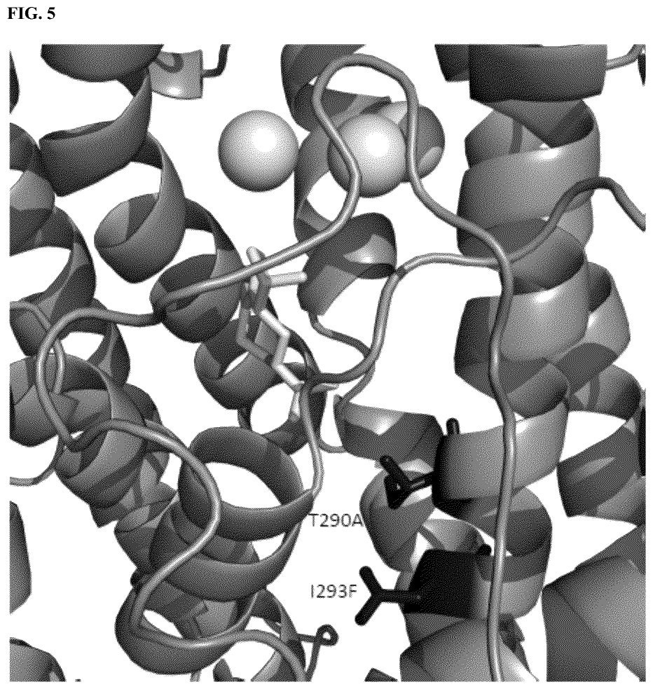

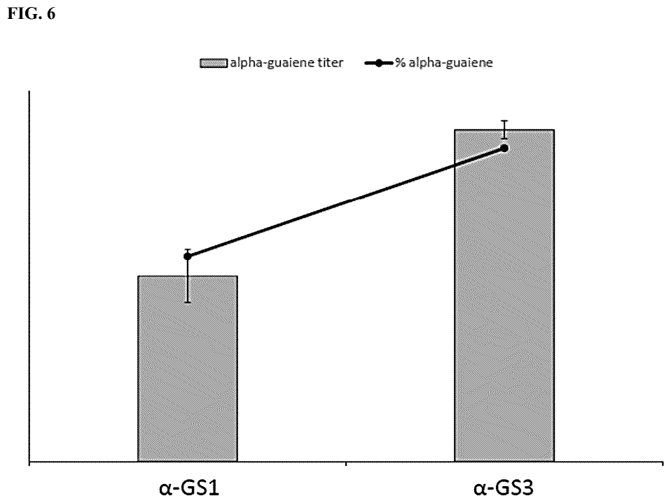

FIG. 6 compares α-Guaiene titer (gray bars) and % α-Guaiene (line) produced by fermentation of E. coli strains expressing αGS1 (SEQ ID NO: 2) or αGS3 (SEQ ID NO: 4) in 96 well plates for 72 hours. αGS3 shows similar improvements in α-Guaiene titer as compared to α-GS2, but αGS3 shows a dramatic improvement in % α-Guaiene.

An additional 174 mutations in αGS3 were screened in Round 4. Amino acid substitutions were evaluated for changes to α-Guaiene titer as well as % α-Guaiene (of total product). The following amino acids substitutions showed significant improvement in one or more of these parameters:

TABLE 3
Round 4 of αGS Engineering

| | Fold improvement | Fold improvement |
|---|---|---|
| Mutation | α-Guaiene titers | % α-Guaiene of total |
| L447V | 2.39 | 1.08 |
| I372L | 2.38 | 1.03 |
| T296V | 2.32 | 1.12 |
| T296I | 2.28 | 1.09 |
| I400V | 2.00 | 1.05 |
| F293I | 1.99 | 0.69 |
| L439K | 1.83 | 0.99 |
| A452T | 1.70 | 1.00 |
| I292L | 1.59 | 0.89 |
| A452Q | 1.58 | 0.90 |
| K480E | 1.54 | 0.91 |
| Q203P | 1.40 | 0.92 |
| F293V | 1.31 | 0.60 |
| F369M | 1.27 | 0.91 |
| E325S | 1.20 | 0.96 |
| K173T | 1.19 | 0.99 |
| C189Q | 1.18 | 0.99 |
| K220H | 1.11 | 0.99 |
| F513M | 1.08 | 1.10 |
| V516M | 1.08 | 1.03 |
| I440L | 1.07 | 0.98 |
| A290G | 1.07 | 0.94 |
| A452D | 1.05 | 0.80 |
| M481L | 1.04 | 0.97 |
| C149S | 1.04 | 0.97 |
| I400L | 1.04 | 1.06 |
| P212S | 1.03 | 0.93 |
| I399V | 1.02 | 0.99 |
| R172T | 1.00 | 0.99 |
| M273I | 0.63 | 1.33 |

Several mutations were identified that provided substantial improvements to α-Guaiene titer, while providing modest improvements, no improvement, or only modest impacts in % α-Guaiene. The M273I mutation resulted in a substantially improved % α-Guaiene. FIG. 7 illustrates a homology model of αGS4 (SEQ ID NO: 5). Three amino acid substitutions (M273L, I400L, and L447V) were selected during Round 4 engineering. Substitution M273L may alter the distance of C helix to INT5 and favor the deprotonation of INT 5 to α-Guaiene. These substitutions were added to αGS3 to create αGS4.

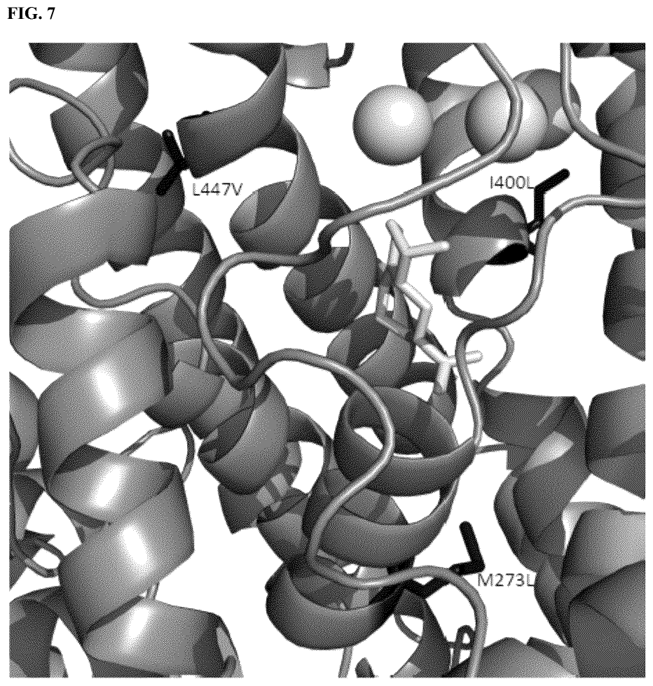

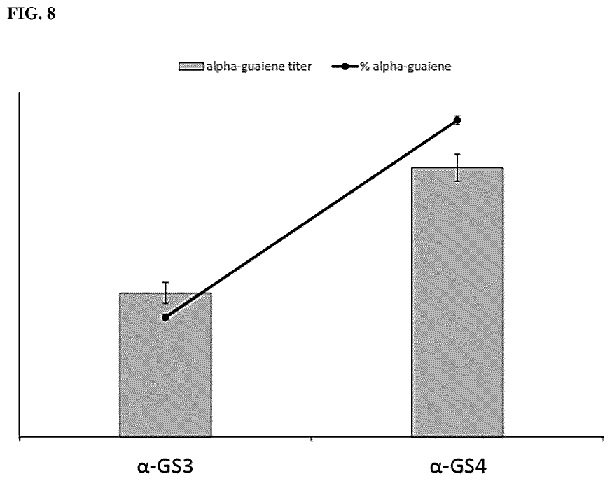

FIG. 8 compares α-Guaiene titer (gray bars) and % α-Guaiene (line) produced by fermentation of E. coli strains expressing αGS3 (SEQ ID NO: 3) or αGS4 (SEQ ID NO: 5) in 96 well plates for 72 hours. Compared to αGS3, αGS4 resulted in both a substantially improved α-Guaiene titer, as well as a substantially improved profile (% α-Guaiene).

In summary, the design of αGS4 contains several mutations (with respect to SEQ ID NO: 1) that are believed to shift the profile towards α-Guaiene: F407L, T290A, I293F, and M273I. αGS4 further contains several mutations (with respect to SEQ ID NO: 1) that are believed to improve overall α-Guaiene titer without shifting profile significantly: S375A, Y443L, I400V, and L447V. It is notable that all of the mutations were identified in the C-terminal domain of the terpene synthase (258 to 548 of SEQ ID NO:1). The C-terminal domain, which harbors the active site, is therefore most critical for its enzymatic activity.

For Round 5 of αGS engineering using in vivo production of αGS4 mutants, via fermentation performed in a 96 well plate for 72 hours, the following results were obtained.

TABLE 4
Round 5 of αGS Engineering

| | Fold improvement | Fold improvement |
|---|---|---|
| Mutation | α-Guaiene titers | % α-Guaiene of total |
| K332E | 3.13 | 1.28 |
| I292M | 2.32 | 1.18 |
| F293M | 2.31 | 0.71 |
| I485L | 2.09 | 0.95 |
| T24S | 1.94 | 1.18 |
| L284R | 1.87 | 1.19 |
| K480E | 1.84 | 1.02 |
| S390T/Y391F | 1.81 | 0.83 |
| R482K | 1.80 | 1.11 |
| E325T | 1.79 | 1.19 |
| Y271V | 1.78 | 1.02 |
| T296V | 1.78 | 1.15 |
| A462G | 1.77 | 0.97 |
| I292L | 1.74 | 0.99 |
| I292V | 1.73 | 1.10 |
| V312L/T313K/L314I | 1.72 | 1.05 |
| G457K | 1.68 | 0.86 |
| V336P | 1.66 | 1.14 |
| R248T | 1.65 | 1.20 |
| K173T | 1.63 | 1.15 |
| A355K | 1.62 | 1.10 |
| G343D | 1.61 | 1.12 |
| Y271A | 1.61 | 0.72 |
| M520L | 1.60 | 0.83 |
| F348I | 1.57 | 0.94 |
| S504R | 1.56 | 1.08 |
| T429S | 1.56 | 1.05 |
| G340A/G343D | 1.55 | 1.05 |
| V388I | 1.55 | 1.10 |
| E140K | 1.54 | 1.14 |
| I372L | 1.53 | 1.11 |
| Y450R | 1.53 | 0.81 |
| N351F | 1.51 | 1.09 |
| F348L | 1.47 | 1.07 |
| I372V | 1.46 | 1.21 |
| I408L | 1.45 | 0.48 |
| F348M | 1.44 | 1.04 |
| M274L | 1.42 | 1.17 |
| S504M | 1.41 | 1.09 |
| L420T | 1.41 | 1.11 |
| I487D | 1.40 | 1.13 |
| R468K | 1.38 | 1.08 |
| Q448K | 1.37 | 1.06 |
| K220H | 1.36 | 1.07 |
| L298I | 1.36 | 1.11 |
| K281Q | 1.36 | 1.12 |
| Y387H | 1.35 | 1.06 |
| A545P | 1.33 | 1.13 |
| K287E | 1.31 | 1.11 |
| T385E | 1.31 | 1.02 |
| F513L | 1.30 | 1.03 |
| T495E | 1.30 | 1.12 |
| V312L/T313K | 1.28 | 0.75 |
| V463L | 1.28 | 0.99 |
| E455K | 1.27 | 1.11 |
| M481I | 1.27 | 1.12 |
| I399V | 1.27 | 1.00 |
| I409V | 1.26 | 1.09 |
| D359Q | 1.24 | 1.07 |
| V336L | 1.24 | 1.09 |
| I485V | 1.23 | 1.13 |
| T459S/V460S | 1.22 | 0.57 |
| I372T | 1.22 | 1.01 |
| Y382W | 1.22 | 1.13 |
| M481F | 1.21 | 1.04 |
| E325S | 1.17 | 0.91 |
| Q203P | 1.16 | 0.64 |
| K287I | 1.16 | 1.12 |
| L284R/S285G | 1.14 | 1.03 |
| I266L | 1.13 | 1.07 |
| Y271F | 1.13 | 1.08 |
| L289V | 1.13 | 0.95 |
| L289M | 1.12 | 1.06 |
| Q335E | 1.11 | 1.09 |
| L439K | 1.11 | 0.83 |
| K332E/Q335K | 1.10 | 1.08 |
| T302S | 1.09 | 1.04 |
| Q448M | 1.09 | 1.13 |
| R17P/D18I | 1.08 | 0.88 |
| T24S/V25I | 1.08 | 1.08 |
| D359T | 1.06 | 0.86 |
| R356E | 1.05 | 1.10 |
| R374K | 1.03 | 1.03 |
| S390T/Y391T | 1.02 | 1.02 |
| R482Q | 1.02 | 0.96 |
| I444V | 1.02 | 0.95 |

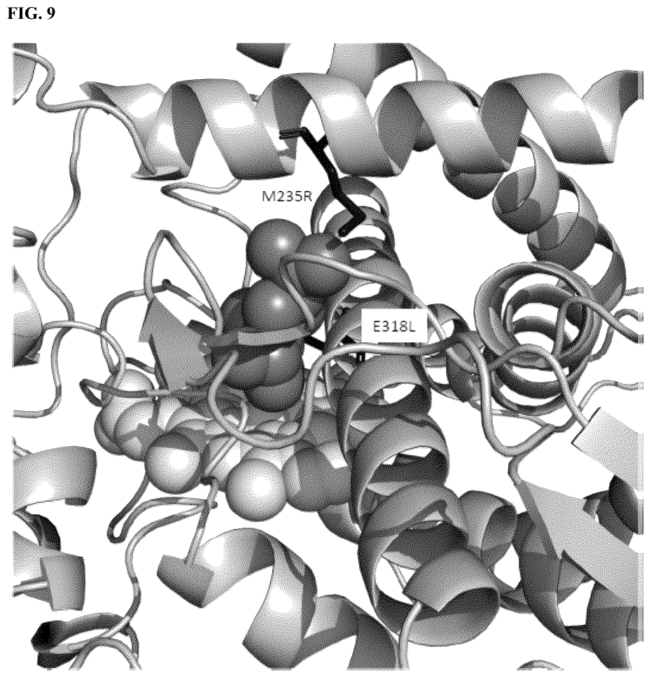

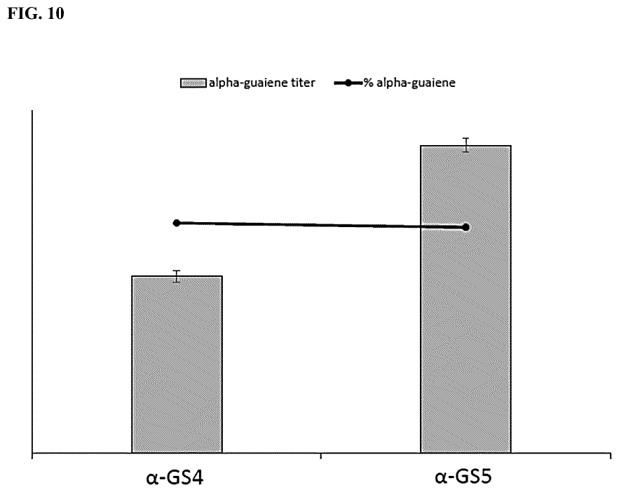

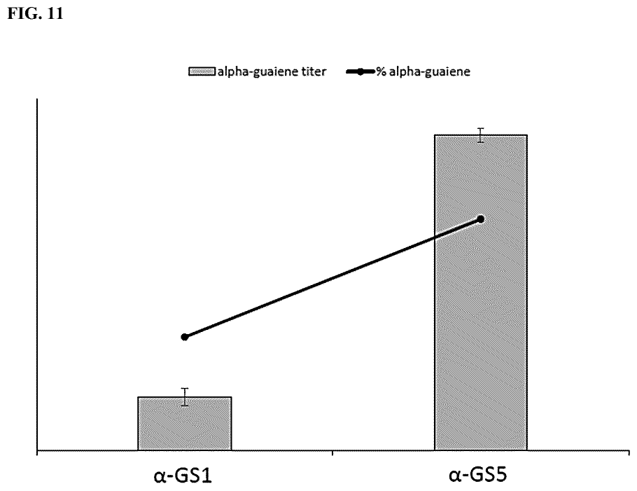

αGS5 (SEQ ID NO: 28) incorporates the mutation T296V/E325T with respect to αGS4 (SEQ ID NO: 5). Improvement in α-Guaiene titers using αGS4 (as compared to αGS4) is shown in FIG. 10. % α-Guaiene remained stable along with a significant improvement in α-Guaiene titer. From αGS1 to αGS5, α-Guaiene titers improve about 5 times, while %-Guaiene improves about 2 times. See FIG. 11.

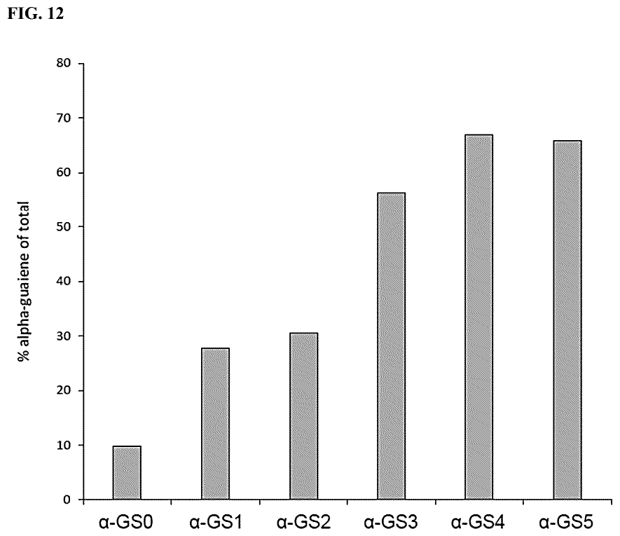

FIG. 12 shows the generations of α-GS as a function of α-Guaiene percent of the total products. In vivo production of α-Guaiene are shown from an engineered E. coli strain expressing α-GS0 through α-GS5. Fermentation was performed in a 96 well plate for either 48 or 72 hours. While αGS0 produces only about 10% α-Guaiene, αGS4 and αGS5 produce about 65% α-Guaiene as a percent of total product.

Additional mutations in αGS5 were screened in Round 6. In vivo production of α-Guaiene by these mutants was evaluated. Fermentation was performed in a 96 well plate for 72 hours. Amino acid substitutions were evaluated for changes to α-Guaiene titer as well as % α-Guaiene (of total product). The amino acid substitutions that showed significant improvement in one or more of these parameters are shown in Table 5 below:

TABLE 5
Round 6 of αGS Engineering

| | Fold Improvement | Fold improvement |
|---|---|---|
| Mutant | α-Guaiene titers | % α-Guaiene of total |
| Q448V | 1.22 | 1.03 |
| A290S | 1.19 | 0.99 |
| Q448M | 1.16 | 1.00 |
| Y271F | 1.16 | 0.98 |
| A545P | 1.14 | 0.97 |
| G275S | 1.13 | 0.98 |
| S15N | 1.13 | 1.00 |
| R248T | 1.13 | 1.00 |
| I487D | 1.10 | 1.00 |
| A267I | 1.10 | 0.96 |
| I440M/F513V | 1.09 | 1.00 |
| Y21F | 1.09 | 1.05 |
| L443Y | 1.06 | 0.95 |
| K332E | 1.06 | 1.00 |
| G275A | 1.06 | 1.01 |
| T24S | 1.06 | 1.00 |
| L412I | 1.05 | 1.00 |
| K47Q | 1.02 | 1.00 |
| L443I | 1.02 | 1.00 |
| F278P | 1.02 | 1.00 |
| N57R | 1.01 | 1.00 |
| N41D | 1.01 | 1.00 |
| I440A/F513V | 1.01 | 1.00 |
| I273L/S401V | 0.98 | 1.01 |
| V373I | 0.93 | 1.02 |
| M274L | 0.91 | 1.01 |
| G269S | 0.89 | 1.07 |
| I399V | 0.88 | 1.01 |
| G269A | 0.87 | 1.05 |
| S43E | 0.87 | 1.01 |
| G307A | 0.86 | 1.01 |

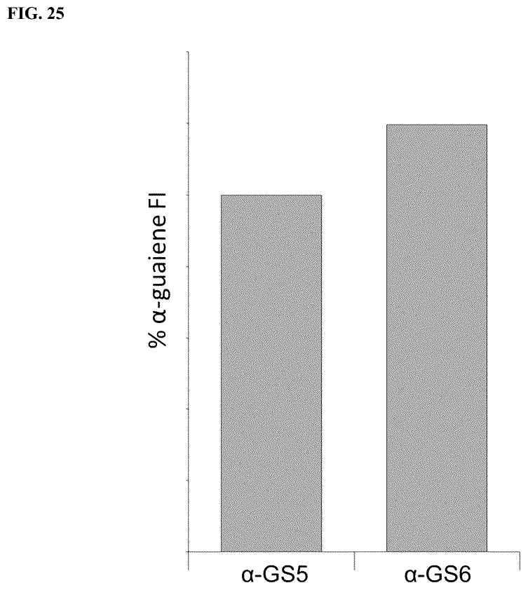

αGS6 (SEQ ID NO: 31) incorporates the mutation G269S/Y21F/Q448V/A545P with respect to αGS5 (SEQ ID NO: 28). Improvement in α-Guaiene titers using αGS6 (as compared to αGS5) is shown in FIG. 25.

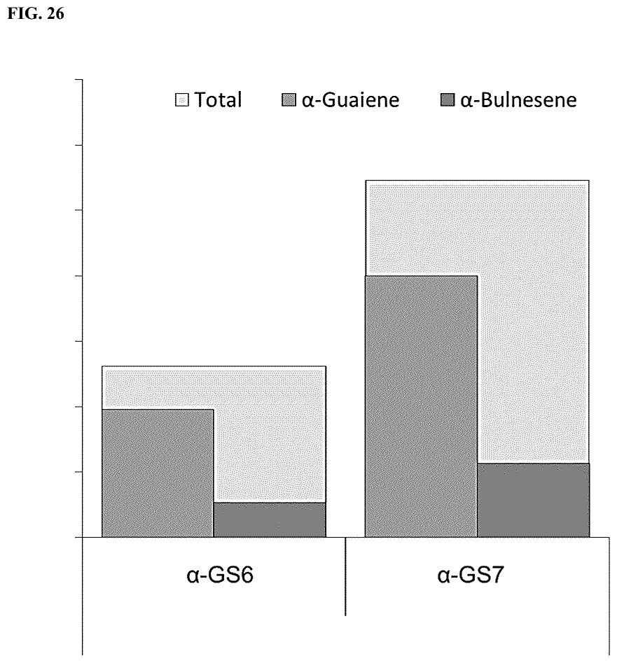

Additional mutants of αGS6 were screened in Round 7 and in vivo production of α-Guaiene by these mutants was evaluated. αGS7 (SEQ ID NO: 32) incorporates the mutation V448Q/1487D with respect to αGS6 (SEQ ID NO: 31). FIG. 26 shows in vivo production of α-Guaiene, α-bulnesene and total cyclized products during fermentation by engineered E. coli strains expressing α-GS6 or α-GS7. Fermentation was performed in a 96 well plate for 72 hours.

### Example 2: Engineering of an α-Guaiene Oxidase Enzyme

A candidate αGO (SEQ ID NO: 6) is disclosed in WO 2020/051488, which is hereby incorporated by reference in its entirety. The αGO is an engineered derivative of a Kaurene Oxidase.

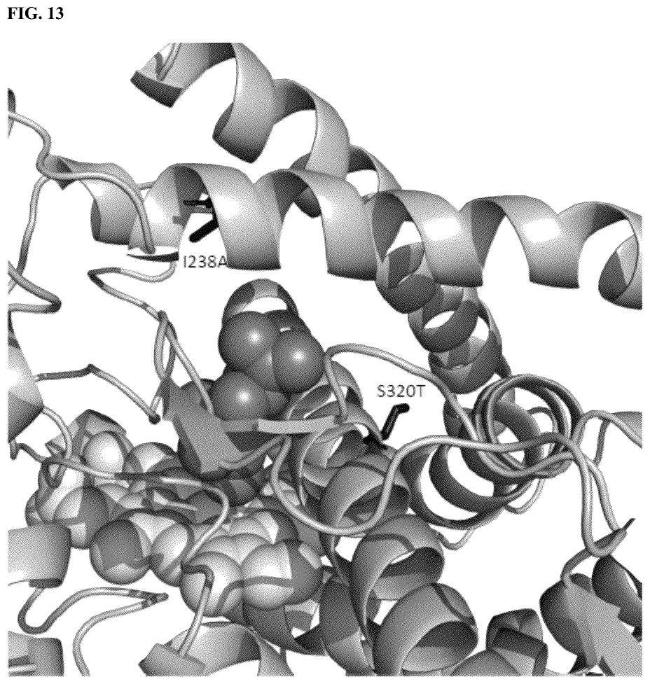

FIG. 13 illustrates a homology model of the αGO, which was used to guide mutations for screening in parallel to αGS engineering. Substrate molecule was docked, and the binding mode was optimized to be consistent with existing in vivo data. Select mutants were expressed in E. coli strains from Example 1, co-expressing αGS1 and a cytochrome P450 reductase (SEQ ID NO: 20). Fermentation was performed in 96-well plates for 72 hours.

191 select mutants were screened and evaluated for improvement in rotundol titers (the first oxygenation event). A list of mutations that provided improvements in rotundol titer are shown in Table 6, along with the impact on titers of side products.

TABLE 6
Round 1 of αGO Engineering

| | Fold improvement | Fold improvement side |
|---|---|---|
| Mutant | rotundol titers | product titers |
| A497R | 1.49 | 1.70 |
| M235T | 1.31 | 1.46 |
| M235R | 1.30 | 1.48 |
| R451K | 1.26 | 1.30 |
| L72W | 1.25 | 1.42 |
| I490G | 1.25 | 1.31 |
| M496K | 1.22 | 1.12 |
| L368I | 1.21 | 1.29 |
| I490D | 1.19 | 1.49 |
| E318A | 1.15 | 2.01 |
| L387S | 1.14 | 1.52 |
| I490F | 1.14 | 1.44 |
| I490Y | 1.14 | 1.33 |
| A497E | 1.13 | 1.84 |
| M496P | 1.13 | 0.87 |
| M235S | 1.12 | 0.98 |
| P386S | 1.10 | 0.96 |
| D134N | 1.09 | 1.01 |
| K498R | 1.08 | 1.04 |
| A497K | 1.08 | 1.25 |
| M235K | 1.08 | 1.10 |
| I252V | 1.07 | 1.04 |
| A497P | 1.07 | 0.93 |
| M235A | 1.06 | 0.92 |
| T489V | 1.06 | 0.88 |
| M496T | 1.05 | 0.88 |
| L387F | 1.05 | 0.94 |
| M235V | 1.05 | 1.11 |
| L387V | 1.04 | 1.02 |
| Q346R | 1.03 | 1.13 |
| E342D | 1.03 | 1.13 |
| T489M | 1.03 | 1.06 |
| M234L | 1.02 | 0.69 |
| P386A | 1.01 | 0.84 |
| T489K | 1.00 | 1.06 |
| L387Q | 1.00 | 1.20 |
| A174E | 0.99 | 0.86 |
| T489Q | 0.97 | 1.11 |
| T489L | 0.97 | 1.13 |
| K498R | 0.97 | 0.83 |
| M496R | 0.97 | 0.81 |
| D488N | 0.96 | 1.22 |
| D134A | 0.96 | 0.93 |
| I490S | 0.96 | 1.10 |
| D258E | 0.95 | 0.89 |
| T489E | 0.93 | 1.04 |
| P386T | 0.92 | 0.98 |
| A497S | 0.92 | 0.71 |
| L387M | 0.91 | 0.85 |
| A497V | 0.91 | 0.57 |
| V168I | 0.91 | 0.71 |
| E318L | 0.91 | 0.33 |
| L387P | 0.90 | 0.64 |
| P115S | 0.90 | 0.85 |
| I317V | 0.89 | 0.70 |
| M235E | 0.88 | 0.85 |
| M496L | 0.87 | 0.67 |
| M235G | 0.86 | 0.68 |
| S239D | 0.85 | 0.83 |
| M130P | 0.85 | 0.79 |
| E318G | 0.83 | 1.45 |
| L387G | 0.81 | 0.92 |
| L387I | 0.81 | 0.53 |
| I490V | 0.81 | 0.91 |
| P386N | 0.80 | 0.53 |
| A497L | 0.80 | 1.08 |
| A497I | 0.78 | 0.91 |
| M235L | 0.78 | 0.81 |
| T489A | 0.78 | 1.12 |
| D134T | 0.75 | 0.83 |

Two amino acid substitutions (M235R and E318L) were identified in Round 1 engineering, these substitutions incorporated into the enzyme to prepare αGO1 (SEQ ID NO: 7). Substitution E318L may bring the substrate closer to the heme reaction center to favor the major products rotundone and rotundol. See FIG. 13.

For further engineering of the αGO, 173 select mutants were screened, and evaluated for improvement in rotundol and rotundone titers. Select mutants were expressed in E. coli strains as above, with addition of dehydrogenase expression. A list of mutations that provided improvements in rotundol or rotundone titer are shown in Table 7.

TABLE 7
Round 2 of αGO Engineering

| | Fold improvement | Fold improvement |
|---|---|---|
| Mutation | rotundol titers | rotundone titers |
| S311L | 108.47 | 6.79 |
| D134G | 77.74 | 5.70 |
| K250R | 67.99 | 5.05 |
| S350E | 67.43 | 5.22 |
| L318G | 66.72 | 2.13 |
| M324V | 65.91 | 5.18 |
| L145M | 62.68 | 6.18 |
| L312I | 60.74 | 6.16 |
| L308V | 60.72 | 4.87 |
| D134N | 60.50 | 5.07 |
| I317L | 60.10 | 0.02 |
| L308I | 57.30 | 5.53 |
| I238A | 56.52 | 7.05 |
| Q227E | 56.39 | 4.63 |
| S350N | 54.22 | 6.43 |
| L312V | 53.73 | 5.54 |
| K250S | 52.14 | 4.76 |
| S350K | 51.06 | 5.43 |
| R201K | 46.82 | 5.42 |
| I238V | 44.93 | 4.88 |
| T323P | 42.98 | 0.00 |
| R343A | 41.89 | 5.36 |
| L226Y | 41.68 | 0.00 |
| K339D | 40.90 | 4.67 |
| Q107S | 40.38 | 0.00 |
| N178D | 34.73 | 6.29 |
| N221S | 34.54 | 3.92 |
| K250N | 33.85 | 4.20 |
| V231S | 31.79 | 5.64 |
| D305E | 31.57 | 5.81 |
| T326T | 30.03 | 5.89 |
| L318A | 29.92 | 12.54 |
| L187F | 29.65 | 5.65 |
| N221G | 29.31 | 4.83 |
| L72M | 29.12 | 5.84 |
| S319G | 28.83 | 4.77 |
| V228A | 27.42 | 3.96 |
| L309I | 26.15 | 0.00 |
| M324S | 26.04 | 4.98 |
| I238L | 25.76 | 4.40 |
| S320A | 25.27 | 4.99 |
| T327V | 24.91 | 7.91 |
| V128L | 23.87 | 0.00 |
| T327S | 23.77 | 3.32 |
| L72W | 23.69 | 5.34 |
| I317V | 23.58 | 0.00 |
| V325A | 22.43 | 3.79 |
| T323A | 22.11 | 0.00 |
| M127A | 21.59 | 0.00 |
| L72I | 20.52 | 2.72 |
| V128I | 20.30 | 0.00 |
| I316T | 20.00 | 0.00 |
| V89M | 18.00 | 3.95 |
| M127V | 16.76 | 3.69 |
| M127T | 16.61 | 0.00 |
| T323V | 16.48 | 2.71 |
| Q306D | 16.40 | 3.96 |
| I316L | 15.43 | 0.00 |
| L187F_2 | 15.41 | 0.00 |
| V325I | 15.09 | 4.49 |
| S320T | 14.80 | 6.40 |
| T326A | 14.59 | 0.00 |
| S319I | 14.57 | 0.00 |
| I490G | 14.21 | 6.91 |
| L72R | 13.99 | 0.00 |
| I439A | 13.35 | 5.55 |
| P315V | 13.15 | 0.00 |
| S350R | 12.91 | 4.05 |
| H361D | 12.70 | 4.88 |
| M127L | 12.68 | 0.00 |
| P315T | 12.31 | 0.00 |
| S363C | 12.19 | 5.07 |
| L72F | 11.89 | 0.00 |
| I316V | 11.70 | 0.00 |
| Q306K | 11.49 | 5.05 |
| S363A | 11.42 | 5.57 |
| L145V | 11.38 | 0.00 |
| I439M | 10.80 | 5.51 |
| I384V | 10.77 | 5.99 |
| F175L | 10.76 | 5.15 |
| P315S | 10.31 | 4.63 |
| E486K | 10.30 | 5.21 |
| I439Y | 10.24 | 5.44 |
| V231T | 10.21 | 4.75 |
| K250Q | 10.05 | 3.37 |
| S319A | 9.99 | 4.40 |
| V231A | 9.92 | 5.25 |
| G491Q | 9.80 | 5.60 |
| E486D | 9.57 | 4.80 |
| E360D | 9.07 | 1.35 |
| T260R | 8.91 | 0.00 |
| T326V | 8.59 | 0.00 |
| S319T | 7.81 | 2.92 |
| S363P | 7.47 | 4.12 |
| T122S | 6.83 | 3.58 |
| V231L | 6.59 | 3.41 |
| S465C | 6.36 | 5.13 |
| I371V | 5.80 | 4.72 |
| L312A | 5.41 | 2.26 |
| A497L | 5.37 | 3.86 |
| T489K | 5.32 | 5.57 |
| L461M | 5.20 | 4.70 |
| G491K | 4.79 | 4.09 |
| L492F | 4.52 | 4.17 |
| T483E | 4.50 | 2.74 |
| I490S | 4.28 | 2.55 |
| T483D | 3.96 | 2.44 |
| H400L | 3.86 | 2.30 |
| R235T | 3.83 | 2.38 |
| T260K | 3.79 | 3.66 |
| T489Q | 3.67 | 2.22 |
| G467A | 3.48 | 2.00 |
| V395Q | 3.36 | 2.12 |
| G491L | 3.01 | 1.92 |
| T489M | 2.87 | 4.70 |
| I490K | 2.87 | 0.00 |
| L387S | 2.77 | 1.47 |
| L385M | 2.75 | 3.35 |
| M496K | 2.71 | 2.23 |
| P383A | 2.59 | 2.93 |
| A497E | 2.48 | 2.75 |
| I384M | 2.48 | 1.50 |
| T493M | 2.34 | 0.00 |
| G491C | 2.22 | 1.58 |
| G491A | 2.15 | 1.55 |
| Q442Y | 1.87 | 1.36 |
| A497R | 1.83 | 1.46 |
| E486S | 1.78 | 1.30 |
| K498H | 1.69 | 1.26 |
| R451K | 1.62 | 1.60 |
| V325L | 1.40 | 2.22 |
| V487I | 1.39 | 1.25 |
| L368I | 1.19 | 1.04 |
| L387G | 1.17 | 2.46 |
| L368V | 1.16 | 1.20 |

Co-expression of the dehydrogenase along with GO engineering very significantly improves titer of the oxygenated products rotundol and rotundone. Two amino acid substitutions (I238A and S320T) were selected during Round 2 engineering (illustrated in FIG. 12) to prepare αGO2 (SEQ ID NO: 8).

170 select mutants were screened and evaluated for improvement in rotundol and rotundone titers. Select mutants were expressed in E. coli strains as above with dehydrogenase co-expression. A list of mutations that provided improvements in rotundol or rotundone titer are shown in Table 8.

TABLE 8
Round 3 of αGO Engineering

| | Fold | Fold |
|---|---|---|
| | improvement | improvement |
| | rotundol | rotundone |
| Mutation | titers | titers |
| L318A | 0.24 | 2.38 |
| L318G | 0.20 | 2.16 |
| T493S | 1.34 | 1.99 |
| L318A/T320S//I490G | 0.21 | 1.85 |
| G491Y | 1.54 | 1.54 |
| G491T | 1.66 | 1.45 |
| L492V | 1.52 | 1.43 |
| I371T | 1.51 | 1.43 |
| V231I | 1.14 | 1.37 |
| V395K | 1.11 | 1.32 |
| V231E | 1.32 | 1.31 |
| V487E | 1.25 | 1.31 |
| P315A | 1.16 | 1.29 |
| I371C | 1.18 | 1.27 |
| I371A | 1.26 | 1.27 |
| I371V | 1.24 | 1.26 |
| L461M | 1.28 | 1.25 |
| V395I | 1.40 | 1.25 |
| P315L | 1.08 | 1.24 |
| G491R | 1.23 | 1.24 |
| H400L | 1.27 | 1.24 |
| Q126L | 1.01 | 1.23 |
| V395T | 1.00 | 1.23 |
| G491L | 1.17 | 1.22 |
| V231A | 1.26 | 1.22 |
| D215E | 1.32 | 1.21 |
| T483Q | 1.33 | 1.21 |
| P223L | 1.27 | 1.20 |
| L461Y | 1.24 | 1.20 |
| L72F | 1.03 | 1.19 |
| D244E | 1.16 | 1.18 |
| N419K | 1.30 | 1.17 |
| P315S | 0.71 | 1.16 |
| T483N | 1.22 | 1.16 |
| N178D_A238I | 1.02 | 1.14 |
| L187F_A238I_T320S | 1.26 | 1.13 |
| A238T | 1.12 | 1.13 |
| M324F | 1.14 | 1.12 |
| M324N | 1.16 | 1.12 |
| I490K | 0.95 | 1.12 |
| L318S | 0.70 | 1.12 |
| M324T | 0.98 | 1.12 |
| V395N | 1.22 | 1.11 |
| V231Q | 1.25 | 1.11 |
| V231G | 1.04 | 1.11 |
| M324G | 1.00 | 1.10 |
| L461I | 1.15 | 1.10 |
| L145V | 1.00 | 1.10 |
| Q107S_I490S | 1.00 | 1.10 |
| M433L | 1.26 | 1.09 |
| G467A | 1.13 | 1.08 |
| L461E | 1.19 | 1.07 |
| A238N_T320S | 1.14 | 1.06 |
| E224L | 1.18 | 1.05 |
| T327F | 1.10 | 1.05 |
| T260R | 1.11 | 1.02 |
| L312T | 1.18 | 1.02 |

The three amino acid substitutions L318A/T320S/1490G were selected for GO3 (SEQ ID NO: 9).

For Round 4 of GO Engineering, in vivo production of rotundol (both isomers, referred to as isomer 1 and isomer 2 below) and rotundone from GO3 (SEQ ID NO: 9) mutants co-expressing αGS3 (SEQ ID NO: 4), a CPR (SEQ ID NO: 20), and an ADH (SEQ ID NO: 10) were evaluated. Fermentation was performed in a 96 well plate for 72 hours. Results are summarized in Table 9.

TABLE 9
Round 4 of αGO Engineering

| | Fold | Fold | |
|---|---|---|---|
| | improvement | improvement | Fold |
| | rotundol isomer | rotundol isomer | improvement |
| Mutant | 1 titers | 2 titers | rotundone |
| T489Q/H495S | 1.13 | 1.03 | 1.60 |
| G491S | 0.99 | 1.00 | 1.48 |
| T489N | 1.26 | 1.12 | 1.39 |
| T489S/H495N | 1.45 | 1.32 | 1.39 |
| G491A/L492F | 1.76 | 0.99 | 1.31 |
| T489E | 1.11 | 1.00 | 1.28 |
| E485K/G491L | 1.23 | 1.50 | 1.24 |
| H495R | 1.08 | 1.10 | 1.16 |
| A497K | 1.17 | 1.06 | 1.16 |
| T489N/N494L | 1.02 | 0.95 | 1.14 |
| H495R_2 | 1.07 | 1.08 | 1.14 |
| A497R | 1.16 | 1.07 | 1.13 |
| T489E/H495N | 0.94 | 0.80 | 1.02 |
| GO3 | 0.99 | 1.00 | 1.00 |
| T489E/H495N | 0.99 | 0.87 | 0.97 |
| H495S | 0.73 | 0.65 | 0.94 |
| E485G/G491Y/L492I | 1.03 | 1.36 | 0.86 |
| V487Q/A497K | 0.91 | 1.10 | 0.81 |
| G491T/L492V | 1.07 | 1.00 | 0.65 |
| MB4126 | 0.21 | 2.61 | 0.41 |
| MB4126 | 0.21 | 2.40 | 0.38 |

GO4 (SEQ ID NO: 29) incorporates the mutations T489Q/H495S with respect to GO3 (SEQ ID NO: 9).

For Round 5 of GO Engineering, in vivo production of rotundol (isomer 1 and isomer 2) and rotundone from GO4 (SEQ ID NO 29) mutants co-expressing αGS5 (SEQ ID NO: 28), a CPR (SEQ ID NO: 20), and an ADH (SEQ ID NO: 10) were evaluated. Fermentation was performed in a 96 well plate for 72 hours. Results are summarized in Table 10.

TABLE 10
Round 5 of αGO Engineering

| | Fold | Fold | |
|---|---|---|---|
| | improvement | improvement | Fold |
| | rotundol isomer | rotundol isomer | improvement |
| Mutant | 1 titers | 2 titers | rotundone |
| V240F | 2.38 | 3.32 | 1.21 |
| S239T | 1.61 | 1.7 | 1.14 |
| T371A | 1.43 | 1.44 | 1.14 |
| D440N | 1.05 | 1.43 | 1.13 |
| D440G | 1.14 | 1.08 | 1.1 |
| T371A_S239T | 1.58 | 1.89 | 1.08 |
| N419R | 1.09 | 1.16 | 1.07 |
| L226F | 1.31 | 1.21 | 1.06 |
| S239T | 1.24 | 1.4 | 1.06 |
| K418A | 1.14 | 1.36 | 1.05 |
| K434E_T371A | 1.41 | 1.6 | 1.05 |
| S311T | 0.93 | 0.9 | 1.05 |
| T483Q | 1.13 | 1.2 | 1.05 |
| G449S | 1.41 | 1.55 | 1.04 |
| N419K | 0.94 | 0.84 | 1.04 |
| H400L | 0.72 | 0.66 | 1.03 |
| S457A | 0.91 | 1.32 | 1.02 |
| G449S_S239T | 1.46 | 1.86 | 1.01 |
| K443A | 1.06 | 0.89 | 1.01 |
| Q364K | 1.1 | 1.17 | 1.01 |
| K336R | 1.21 | 1.08 | 1.01 |
| K434E_S239T | 1.64 | 2.04 | 1 |
| I384S | 1.03 | 1.12 | 1 |
| M496L | 2.23 | 5.51 | 1 |
| E426K | 1.01 | 1.17 | 0.99 |
| K434E | 1.03 | 1.15 | 0.99 |
| N423E | 1.05 | 0.97 | 0.98 |
| N419D | 0.89 | 1.15 | 0.98 |
| G449S_T371A | 1.27 | 1.59 | 0.98 |
| K434E | 1.41 | 1.63 | 0.98 |
| K336N | 1.15 | 1.45 | 0.97 |
| K498N | 1.08 | 0.78 | 0.97 |
| K273Q | 1.06 | 0.98 | 0.97 |
| N428R | 0.92 | 1.34 | 0.96 |
| K498Y | 1.13 | 1.2 | 0.96 |
| S320V | 1.11 | 1.09 | 0.96 |
| K434E_G449S | 1.28 | 1.55 | 0.95 |
| P424A | 1.04 | 1.33 | 0.95 |
| L334V | 0.85 | 1.12 | 0.94 |
| V487I | 0.83 | 1.38 | 0.94 |
| V487A | 0.89 | 1.04 | 0.93 |
| G449A | 0.87 | 1.01 | 0.93 |
| K498H | 0.91 | 1.21 | 0.93 |
| K336L | 1.18 | 1.25 | 0.93 |
| E422D | 1.11 | 0.88 | 0.93 |
| K498R | 0.85 | 1.06 | 0.92 |
| K434D | 0.81 | 1.36 | 0.92 |
| A330L | 1.21 | 1.1 | 0.92 |
| K498Q | 0.86 | 1.03 | 0.92 |
| E437K | 0.83 | 1.29 | 0.92 |
| F441L | 1.03 | 1.01 | 0.92 |
| E435D | 0.92 | 1.02 | 0.91 |
| E425D | 0.92 | 1.21 | 0.9 |
| M433I | 1 | 1.26 | 0.9 |
| M433A | 0.9 | 1.22 | 0.9 |
| P315S | 1.07 | 1.22 | 0.9 |
| V240L | 1.01 | 0.85 | 0.89 |
| F432W | 1.01 | 0.85 | 0.89 |
| E437D | 0.79 | 1.1 | 0.88 |
| M433S | 0.77 | 1.15 | 0.87 |
| Q442Y | 0.95 | 1.04 | 0.87 |
| T438K | 0.89 | 1.21 | 0.85 |
| T483N | 0.87 | 1.03 | 0.85 |
| L461M | 0.78 | 1.26 | 0.85 |
| E360S | 0.92 | 1.01 | 0.85 |
| A455I | 1.07 | 0.88 | 0.84 |
| K443E | 1.13 | 0.85 | 0.84 |
| S239A | 0.57 | 1.19 | 0.82 |
| M496K | 1.21 | 3.55 | 0.81 |
| R451K | 1.22 | 3.63 | 0.77 |
| K498D | 0.58 | 1.27 | 0.77 |
| S320I | 1.67 | 2.22 | 0.76 |
| W329F | 1.56 | 1.58 | 0.75 |
| M445L | 0.71 | 1.44 | 0.74 |
| A330I | 1.27 | 1.3 | 0.73 |
| G491S | 0.36 | 1.35 | 0.66 |
| R451Q | 0.89 | 2.49 | 0.54 |
| R451S | 0.91 | 2.83 | 0.5 |
| N494T | 0.72 | 1.17 | 0.48 |
| S320Q | 0.86 | 1.28 | 0.43 |
| M496S | 1.06 | 3.68 | 0.37 |
| K273R | 0.29 | 1.21 | 0.31 |
| A446P | 0.48 | 1.17 | 0.29 |
| M272F | 0.31 | 1.55 | 0.27 |
| D132E | 0.09 | 1.46 | 0.09 |
| D132P | 0 | 1.48 | 0.02 |
| L461Y | 0 | 1.14 | 0 |

GO5 (SEQ ID NO: 30) incorporates the mutation D440G with respect to GO4 (SEQ ID NO: 29).

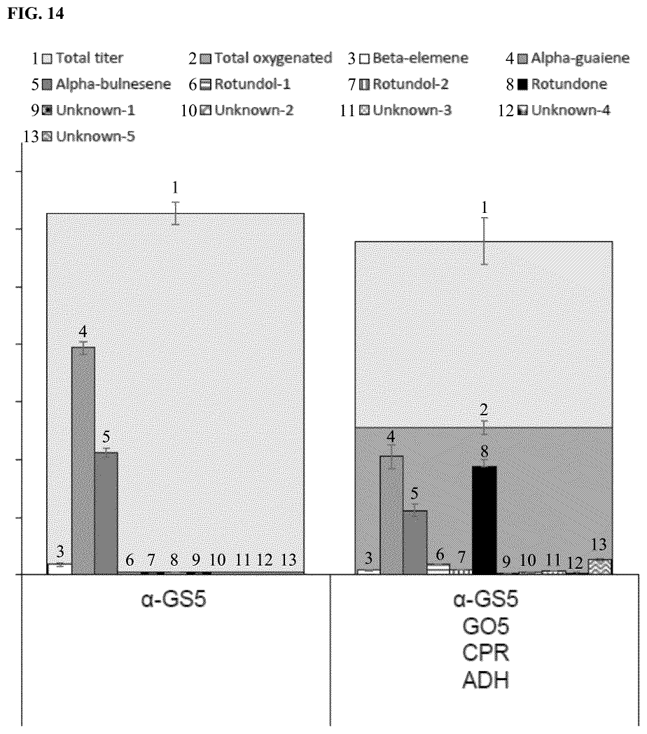

FIG. 14 shows in vivo production of rotundol, rotundone, and other oxygenated products from an engineered E. coli strain co-expressing α-GS5, GO5, a CPR (SEQ ID NO: 20), and an ADH (SEQ ID NO: 10). Fermentation was performed in a 96 well plate for 72 hours. The strain produced rotundone as the main oxygenated product.

A large number of mutants were screened and evaluated for improvement in rotundol and rotundone titers. Briefly, in vivo production of rotundol and rotundone were evaluated by GO5 (SEQ ID NO: 30) mutants co-expressing α-GS6 (SEQ ID NO: 31), a CPR (SEQ ID NO: 20), and an ADH (SEQ ID NO: 10). Fermentation was performed in a 96 well plate for 72 hours. A list of mutations that provided improvements in rotundol or rotundone titer are shown in Table 11.

TABLE 11
Round 6 of αGO Engineering

| | Fold | Fold | Fold |
|---|---|---|---|
| | Improvement | Improvement | Improvement |
| | rotundol isomer | rotundol isomer | rotundone |
| Mutant | 1 titers | 2 titers | titers |
| H389Y | 1.21 | 1.43 | 1.13 |
| W427Y | 1.25 | 1.38 | 1.11 |
| E184A | 1.25 | 1.35 | 1.10 |
| L226F | 1.39 | 1.61 | 1.08 |
| K156R | 1.05 | 1.01 | 1.08 |
| A74E | 1.07 | 1.06 | 1.07 |
| R501H | 1.21 | 1.50 | 1.06 |
| K478R | 1.01 | 0.99 | 1.05 |
| I252F | 1.03 | 1.04 | 1.05 |
| V231I | 1.03 | 1.00 | 1.04 |
| Q301K | 1.02 | 1.06 | 1.04 |
| I218T | 0.95 | 0.91 | 1.03 |
| I357V | 1.00 | 1.26 | 1.03 |
| K217G | 0.94 | 0.92 | 1.03 |
| E47A | 1.06 | 1.09 | 1.02 |
| V89I | 1.12 | 1.15 | 1.02 |
| V409I | 1.01 | 1.23 | 1.02 |
| Q484E | 0.99 | 1.07 | 1.01 |
| A34D | 1.03 | 1.03 | 1.01 |
| N423S | 1.01 | 1.03 | 1.01 |
| L53V | 1.08 | 1.09 | 1.01 |
| V240I | 0.97 | 0.98 | 1.01 |
| A34N | 1.07 | 1.07 | 1.01 |
| Q182L | 1.16 | 1.24 | 1.00 |
| T139M | 1.09 | 1.13 | 1.00 |
| T371C | 0.90 | 1.27 | 0.99 |
| S30- | 0.96 | 0.94 | 0.99 |
| M67K | 1.05 | 1.08 | 0.98 |
| D440Q | 0.93 | 1.09 | 0.98 |
| Q306N | 0.99 | 1.24 | 0.97 |
| V240M | 1.22 | 1.31 | 0.96 |
| R235E | 1.63 | 2.22 | 0.94 |
| K349Q | 0.93 | 1.05 | 0.94 |
| Q126C | 0.98 | 1.01 | 0.93 |
| M496L | 1.85 | 1.94 | 0.92 |
| V240L | 1.11 | 1.25 | 0.92 |
| Y332A | 1.55 | 2.38 | 0.91 |
| S311T | 0.90 | 1.06 | 0.91 |
| G491L/A238T | 1.26 | 1.16 | 0.90 |
| V240F | 1.54 | 1.55 | 0.89 |
| T170S | 1.20 | 2.03 | 0.85 |
| D440W | 0.92 | 1.28 | 0.82 |
| K434E | 0.79 | 1.13 | 0.81 |
| S239T/V240F | 1.51 | 1.61 | 0.79 |
| N494L/A318G | 1.04 | 1.28 | 0.77 |
| G449L | 0.85 | 1.31 | 0.76 |
| G449F | 0.83 | 1.03 | 0.72 |
| L492V/I384S | 2.32 | 2.17 | 0.71 |
| A318L/S320A | 0.55 | 2.45 | 0.71 |
| L492V/L387S | 1.96 | 1.76 | 0.70 |
| G491L/S320T | 0.95 | 1.08 | 0.64 |
| T493S/A238T | 1.10 | 1.45 | 0.63 |
| N494L/S320T | 0.81 | 1.10 | 0.61 |
| N494R/A238T | 1.07 | 1.05 | 0.53 |
| M496A | 1.26 | 1.83 | 0.50 |
| M496F | 1.22 | 2.06 | 0.50 |
| A318L/S319T | 0.19 | 4.55 | 0.43 |
| M496K/A318L | 0.16 | 5.72 | 0.40 |
| M496I | 0.72 | 1.30 | 0.39 |
| A318L/S319A | 0.18 | 5.12 | 0.37 |
| M496L/A318L | 0.17 | 6.01 | 0.37 |
| A318L | 0.13 | 4.68 | 0.36 |
| T493S/A318L | 0.23 | 11.55 | 0.34 |
| A318L/S320T | 0.15 | 5.12 | 0.34 |
| S495T/A318G | 1.17 | 0.69 | 0.33 |
| A497K/A318L | 0.17 | 6.56 | 0.31 |
| A318S/S320T | 0.62 | 2.25 | 0.31 |
| L4921/A318L | 0.14 | 7.15 | 0.31 |
| A318E/S320T | 0.58 | 1.88 | 0.30 |
| A318S/S319A | 0.42 | 1.40 | 0.30 |
| A318Q/S320T | 0.18 | 3.52 | 0.29 |
| G491T/A318L | 0.18 | 5.74 | 0.28 |
| A318S/S320A | 0.50 | 1.49 | 0.25 |
| A318E | 0.39 | 1.96 | 0.25 |
| L387S/A318L | 0.00 | 5.72 | 0.25 |
| N494R/A318L | 0.00 | 5.41 | 0.24 |
| A318Q/S319T | 0.14 | 2.68 | 0.23 |
| M496T | 0.54 | 1.21 | 0.23 |
| I384S/A318L | 0.00 | 3.95 | 0.23 |
| L387S/A318S | 0.37 | 1.59 | 0.23 |
| A318E | 0.45 | 2.28 | 0.23 |
| N494L/A318L | 0.00 | 5.20 | 0.22 |
| L492V/A318L | 0.00 | 4.54 | 0.22 |
| S495R/A318L | 0.08 | 4.15 | 0.22 |
| A497R/A318L | 0.11 | 4.17 | 0.21 |

GO6 (SEQ ID NO: 33) incorporates the mutation E184A/H389Y/R501H relative to GO5 (SEQ ID NO: 30).

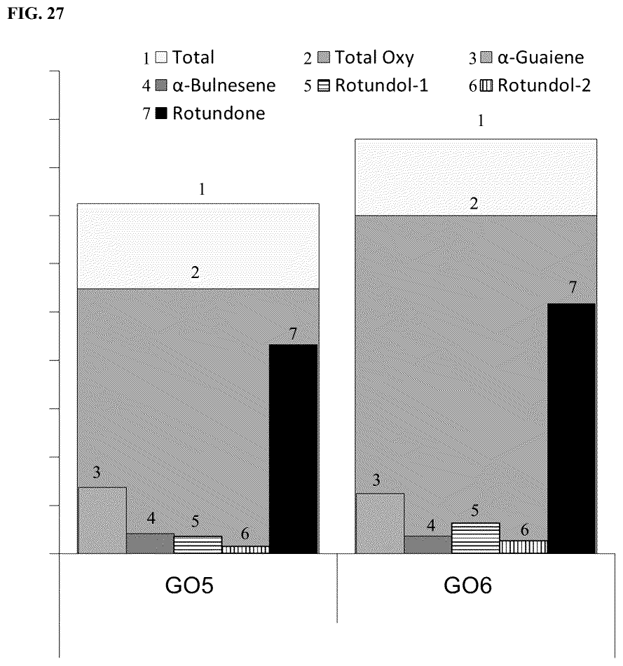

FIG. 27 shows in vivo production of rotundol-1, rotundol-2, rotundone, and total oxygenated products from an engineered E. coli strains co-expressing GO5 or GO6 with α-GS7 (SEQ ID NO: 32), a CPR (SEQ ID NO: 20), and an ADH (SEQ ID NO: 10). Fermentations were performed in a 96 well plate for 72 hours. The strain expressing GO6 showed a futher increase in total oxygenated products and rotundone compared to the strain expressing GO5 (FIG. 27). The strain expressing GO6 showed a decrease in the production of the rotundol byproducts.

A large number of mutants were screened and evaluated for improvement in rotundol and rotundone titers. Briefly, in vivo production of rotundol by GO6 (SEQ ID NO: 33) mutants co-expressing α-GS6 (SEQ ID NO: 31), a CPR (SEQ ID NO: 20), and an ADH (SEQ ID NO: 10) was anlyzed. Fermentation was performed in a 96 well plate for 72 hours. A list of mutations that provided improvements in rotundol or rotundone titer are shown in Table 12.

TABLE 12
Round 7 of αGO Engineering

| | Fold | Fold | Fold |
|---|---|---|---|
| | Improvement | Improvement | Improvement |
| | rotundol isomer | rotundol isomer | rotundone |
| Mutant | 1 titers | 2 titers | titers |
| A284E | 1.18 | 1.09 | 1.27 |
| V390I | 0.56 | 1.31 | 1.19 |
| A446S | 1.02 | 0.87 | 1.17 |
| L226F | 1.36 | 1.31 | 1.14 |
| T84L/V240I | 1.41 | 1.50 | 1.13 |
| E224D | 1.20 | 1.15 | 1.11 |
| Y389F | 1.34 | 1.05 | 1.09 |
| K443R | 0.98 | 0.91 | 1.08 |
| G449A | 1.09 | 1.23 | 1.07 |
| K281E/A284K | 1.08 | 1.09 | 1.07 |
| L387P | 1.55 | 1.28 | 1.07 |
| Q171V/F175V | 1.22 | 1.21 | 1.07 |
| E224N | 1.07 | 1.06 | 1.04 |
| A123F | 1.11 | 1.06 | 1.04 |
| L249M | 1.14 | 1.13 | 1.04 |
| V231H | 1.29 | 1.32 | 1.03 |
| I266E | 1.03 | 1.05 | 1.03 |
| V231T | 1.16 | 1.16 | 1.03 |
| S320T | 1.48 | 1.08 | 1.02 |
| M482D | 1.05 | 1.07 | 1.02 |
| V240F | 1.62 | 1.77 | 1.02 |
| I218R/M220L | 1.13 | 1.12 | 1.01 |
| V231A | 1.17 | 1.24 | 1.01 |
| H137W | 1.04 | 1.06 | 1.01 |
| K116N | 1.15 | 1.13 | 1.01 |
| M130F | 1.09 | 1.15 | 1.00 |
| N259K/T260K/Q263E | 1.08 | 1.11 | 1.00 |
| A318L/S320T | 0.24 | 11.29 | 0.01 |
| A318L | 0.25 | 10.92 | 0.01 |
| A318L/S319T | 0.22 | 9.69 | 0.01 |
| A318L/S320A | 0.24 | 9.67 | 0.02 |
| L387S/A318L | 0.26 | 9.10 | 0.02 |
| A318L/S319A | 0.20 | 8.47 | 0.02 |
| A318Q/S320T | 0.33 | 8.00 | 0.03 |
| A238T/A318L | 0.17 | 7.08 | 0.02 |
| A318Q/S319T | 0.23 | 6.66 | 0.03 |
| L492I/A318L | 0.16 | 6.63 | 0.02 |
| I384S/A318L | 0.18 | 6.57 | 0.03 |
| A318S/S320T | 0.99 | 5.99 | 0.14 |
| T493S/A318L | 0.16 | 5.60 | 0.00 |
| A497K/A318L | 0.19 | 5.11 | 0.02 |
| A318E/S320T | 0.77 | 4.07 | 0.10 |
| A318E | 0.57 | 4.04 | 0.06 |
| S495T/A318L | 0.21 | 3.99 | 0.02 |
| S320I | 1.88 | 3.46 | 0.51 |
| G491Y/A318L | 0.17 | 3.43 | 0.02 |
| L387S/A318S | 0.74 | 3.15 | 0.10 |
| M234A | 0.51 | 3.07 | 0.24 |
| S495R/A318L | 0.13 | 3.07 | 0.00 |
| G491T/A318L | 0.10 | 2.89 | 0.02 |
| N494L/A318L | 0.09 | 2.89 | 0.00 |
| A497R/A318L | 0.11 | 2.84 | 0.02 |
| L492V/A318L | 0.08 | 2.81 | 0.01 |
| G491L/A318L | 0.15 | 2.76 | 0.00 |
| K498Y/A318L | 0.09 | 2.75 | 0.02 |
| M496K/A318L | 0.10 | 2.74 | 0.02 |
| A318S/S320A | 0.71 | 2.50 | 0.06 |
| N494R/A318L | 0.09 | 2.50 | 0.00 |
| V22L | 1.03 | 2.00 | 0.27 |
| G236A/M264I | 1.11 | 2.00 | 0.35 |
| M234I | 1.15 | 1.99 | 0.95 |
| Y332A | 1.59 | 1.86 | 0.71 |
| T84L | 0.97 | 1.73 | 0.36 |
| A237V | 1.11 | 1.72 | 0.74 |
| V231R/R235E/S320V/ | 0.33 | 1.64 | 0.30 |
| D321A | | | |
| M234L | 1.30 | 1.64 | 0.78 |
| G150S/P151D | 1.12 | 1.59 | 0.58 |
| M234K | 0.25 | 1.58 | 0.05 |
| S81L | 0.85 | 1.54 | 0.33 |
| M234R | 0.25 | 1.54 | 0.05 |
| V231L | 1.28 | 1.53 | 0.97 |
| M496L | 1.24 | 1.52 | 0.78 |
| L492I | 1.53 | 1.51 | 0.89 |
| L492A | 2.79 | 1.49 | 0.39 |
| S239T/V240F | 1.29 | 1.45 | 0.88 |
| H373K | 1.43 | 1.44 | 0.60 |
| A455P | 1.27 | 1.43 | 0.95 |
| R235Q | 1.26 | 1.43 | 0.99 |
| V390A | 1.52 | 1.40 | 0.87 |
| A237G | 0.85 | 1.37 | 0.67 |
| Y136R | 1.19 | 1.37 | 0.96 |
| V453F | 1.13 | 1.36 | 0.84 |
| A455V | 1.25 | 1.34 | 0.97 |
| M496F | 0.65 | 1.31 | 0.16 |
| K349H/C352T/S354G/ | 1.42 | 1.29 | 0.93 |
| E355N | | | |
| L387V | 1.49 | 1.28 | 0.98 |
| T369L/H373W/E437R/ | 0.91 | 1.25 | 0.52 |
| I439T/F441E | | | |
| M234F | 1.64 | 1.22 | 0.68 |
| V453M | 0.86 | 1.21 | 0.61 |
| R451K | 1.03 | 1.21 | 0.95 |
| T170S | 1.11 | 1.20 | 0.88 |
| M127G/M130F | 1.07 | 1.19 | 0.91 |
| S169V | 1.14 | 1.17 | 0.94 |
| W427Y | 1.05 | 1.17 | 0.92 |
| E437R | 0.99 | 1.15 | 0.81 |
| L492V/I384S | 1.50 | 1.13 | 0.57 |
| L249V | 1.11 | 1.13 | 0.97 |
| A455S | 0.97 | 1.12 | 0.84 |
| Q3Q | 1.07 | 1.12 | 0.86 |
| G236A | 1.01 | 1.12 | 0.40 |
| L114M | 1.04 | 1.11 | 0.95 |
| A123H | 1.01 | 1.11 | 0.95 |
| Q489M | 1.10 | 1.08 | 0.97 |
| A117S | 1.00 | 1.07 | 0.96 |
| L387I | 1.18 | 1.06 | 0.74 |
| 197V | 0.47 | 1.06 | 1.00 |
| N337D/K339R/L340W | 1.24 | 1.05 | 0.88 |
| I163E/N167R | 1.09 | 1.05 | 0.96 |
| D321I/Q192L/S320V | 0.34 | 1.04 | 0.18 |
| H137W/K141R | 1.02 | 1.03 | 0.93 |
| M220L | 1.00 | 1.03 | 0.83 |
| L492F | 1.56 | 0.88 | 0.84 |
| L387S | 1.23 | 1.00 | 0.93 |
| N95V | 1.09 | 1.00 | 0.99 |
| A408T | 1.02 | 0.92 | 0.92 |
| D207P | 1.01 | 0.93 | 0.99 |

These results demonstrate that many mutations provide a further improvement in rotundol and rotundone titers.

### Example 3: Terpene Synthase Reaction Mechanism and Enzyme Engineering

The proposed reaction mechanism for the conversion of C15 farnesyl diphosphate (FPP) to α-Guaiene is illustrated in FIG. 1A. Initially, three magnesium ions provide the electrophilic driving force to trigger diphosphate ionization that generates a carbocation intermediate (E,Z)-farnesyl cation, which further undergoes a series of different cyclization and hydride shifts. The formed carbocation intermediates can be quenched by nucleophiles to yield final product. In the enzyme pocket, a residue side chain such as histidine or tyrosine can act as a nucleophile to deprotonate a carbocation intermediate at a specific position to produce a particular final product.

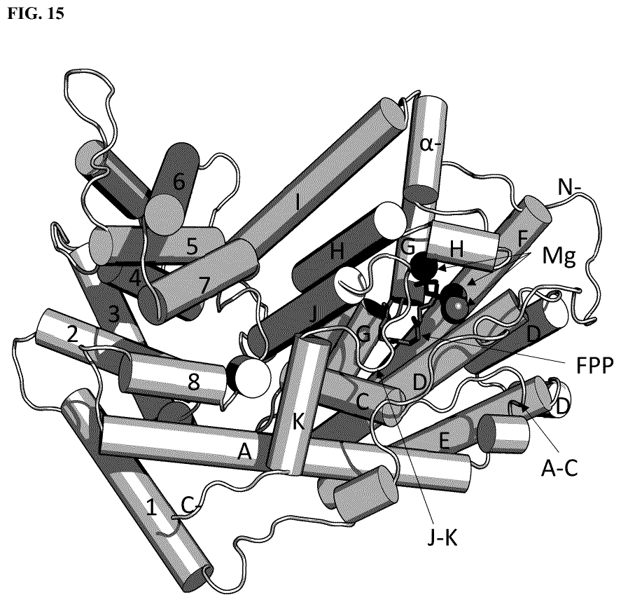

As shown in FIG. 15, either INT5 deprotonated at C2 or INT6 deprotonated at C6 could form α-Guaiene. Either INT4 deprotonated at C6 or INT5 deprotonated at C7 could form α-Bulnesene. Hence, stabilization of intermediates INT5 and INT6 (versus INT4) will lead to favorable α-Guaiene production.

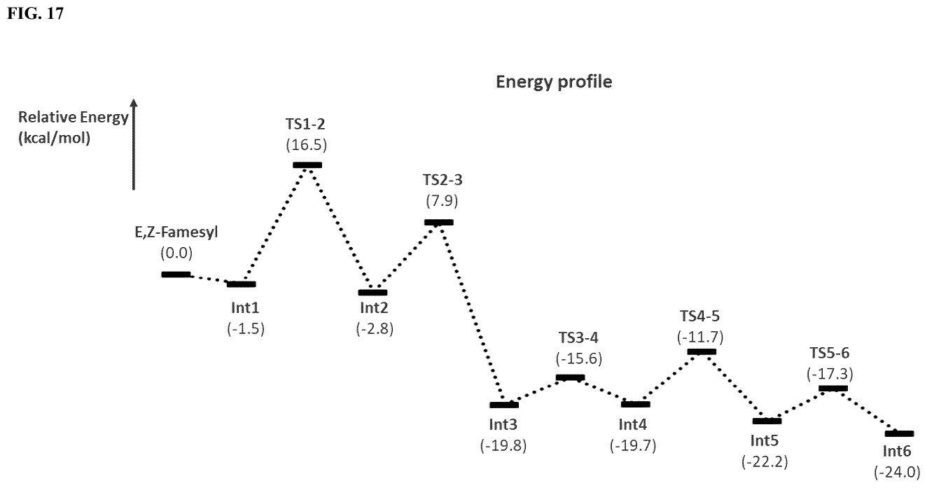

The computed energy profile diagram is presented in FIG. 17. All structures are built with Avogadro software and optimized with NWChem at the B3LYP/6-31G* level. The first step, from INT1 to INT2, is rate-limiting given its relative high energy barrier. INT3 is a much more stable intermediate compared with INT1 and INT2. The energy barriers between INT3 to INT6 are relatively small (<10 kcal/mol), which could indicate that these four intermediates are interconvertible isomers at room temperature when they are not restricted by enzyme residues structurally or electronically.

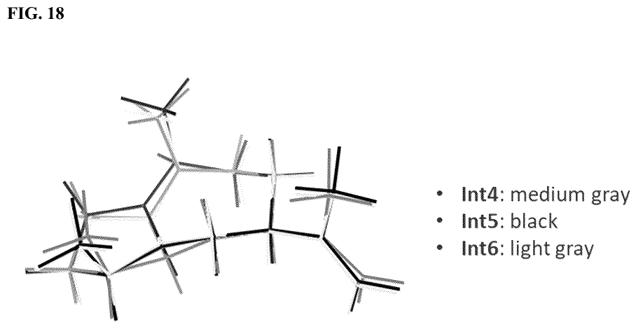

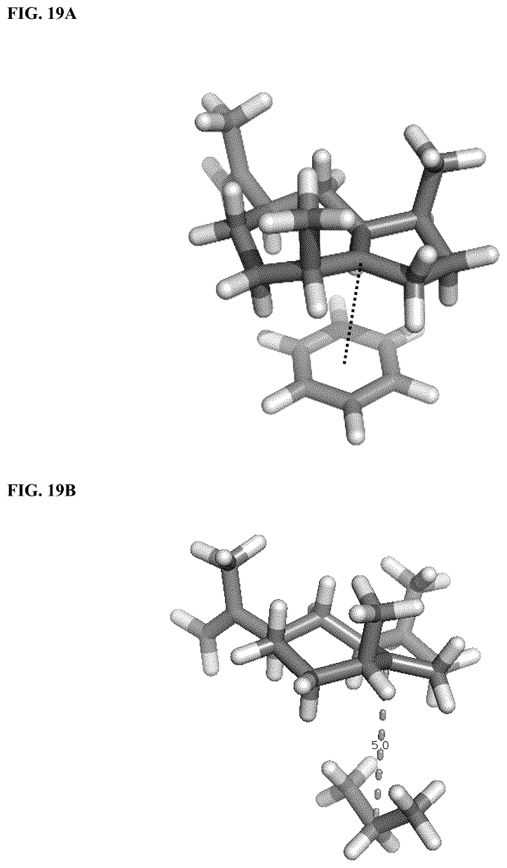

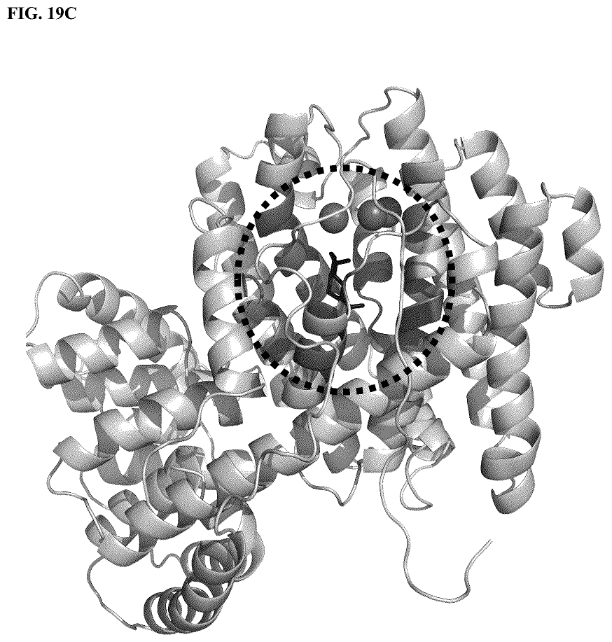

Among the intermediates, INT4 to INT6 are closely related to the desired product α-Guaiene or the main side product α-Bulnesene (FIG. 16). In FIG. 18, superimposed structures of INT4, INT5, and INT6 show that these structures are very similar, which suggests that it will be difficult to stabilize one of them by using steric restrictions given by the enzyme structure alone. Therefore, the enzyme was engineered by stabilizing essential intermediates through direct interaction with enzyme residues, in particular using cation-π interactions to stabilize the desired carbocation. For example, INT5 could be stabilized through a cation-π interaction with benzene molecule by about 6 kcal/mol. A model for this interaction with an aromatic residue is shown in FIG. 19A. INT5 could only be stabilized by ˜1.5 kcal/mol with propane (a model for interaction with an aliphatic residue is shown in FIG. 19B). As the energy difference of INT3 to INT6 is only 5 kcal/mol, this stabilization energy is greater than the energy difference between INT4, INT5, and INT6. Therefore, modifying cation-π interactions has the potential to significantly control the product distribution.

To select potentially beneficial mutations, the INT5 structure was docked onto the GS homology model. In order to change the product profile of the GS enzyme, residues in the substrate binding pocket were targeted as these directly interact with the substrate. Residues on the backside of the helices in the binding pocket were also targeted if they potentially modify positioning of residues in the pocket through indirect interactions. Hence, we selected residues within 10 Å distance from INT5 for protein engineering, as shown in FIG. 19C. Targeted mutagenesis was applied for the selected residues to introduce, remove, or modify cation-π interactions with the substrate.

While residues around the metal-binding motifs are generally conserved among terpene synthases, the hydrophobic bottom of the substrate binding pocket formed by helix C, D, G2 and J play important roles in stabilizing and destabilizing specific intermediates, which will lead to altered product profile. Therefore, mutagenesis focused on primarily on helices C, D, G2 and J. Table 13 shows the secondary structure elements of the GS0 enzyme according to the homology model (See also FIG. 15).

TABLE 13
Secondary Structures and Corresponding Positions in GS0

| Name | Start | End |
|---|---|---|
| Helix 1 | N41 | V61 |
| Helix 2 | P67 | L79 |
| Helix 3 | E86 | L103 |
| Helix 4 | T110 | H120 |
| Helix 5 | A144 | H155 |
| Helix 6 | D162 | L182 |
| Helix 7 | F184 | H195 |
| Helix 8 | Q203 | E217 |
| Helix A | I221 | F253 |
| A-C loop | D254 | I266 |
| Helix C | A267 | A276 |
| Helix D | S283 | V305 |
| Helix D1 | E310 | R321 |
| Helix D2 | I324 | I330 |
| Helix E | P331 | R356 |
| Helix F | I362 | T385 |
| Helix G1 | D392 | I400 |
| Helix G2 | Y404 | M411 |
| Helix H1 | E422 | T429 |
| Helix H2 | P431 | T449 |
| Helix H3 | K451 | R456 |
| Helix α-1 | A462 | F470 |
| Helix I | E475 | L497 |
| Helix J | V507 | D522 |
| J-K Loop | K523 | V533 |
| Helix K | T534 | R543 |

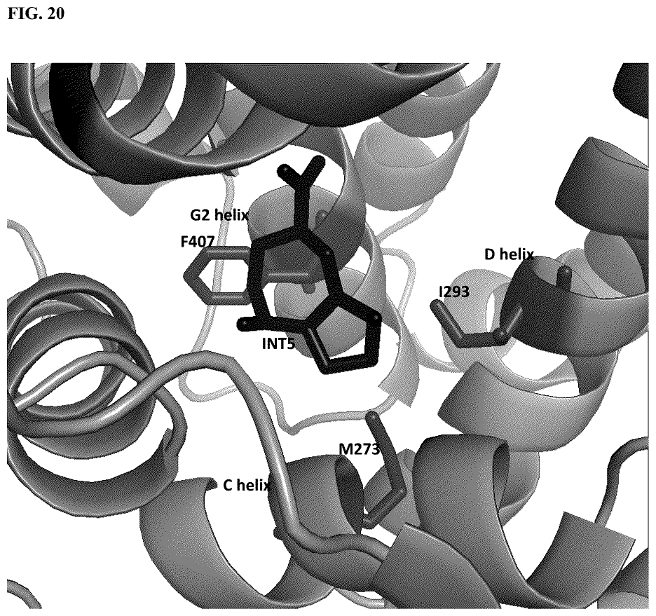

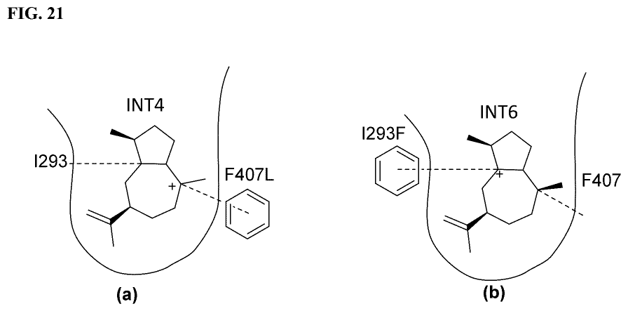

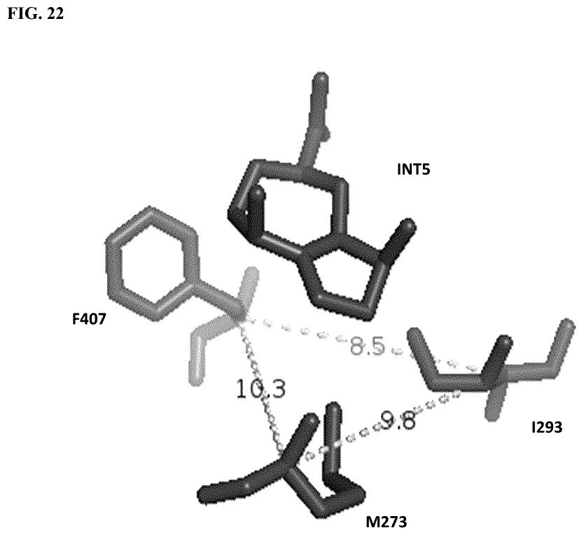

In the homology model, the G2, D, J, and C helices of GS0 interact with each other and form the bottom of the substrate binding pocket (FIGS. 15 and 20). Among them, F407 on the G2 helix could interact with C7 of the intermediates (carbocation location for INT4), and I293 in the D helix could interact with C2 on the intermediates (carbocation location for INT6). Therefore, mutation F407L may destabilize INT4 by removing the cation-π interaction between C7 and phenol ring of F407 (FIG. 21A). Consequently, this could reduce the formation of α-Bulnesene. On the other hand, mutation I293F may stabilize INT6 by adding the cation-π interaction between C2 and phenol ring of F293 (FIG. 21B), which could favor the formation of α-Guaiene. Additionally, mutation M273I in the C helix (FIG. 22) may slightly relocate the intermediate to the more optimal location to interact with F293, in order to stabilize INT6. The combination of the two key mutations (I293F, F407L) should favor INT6 over INT4, leading to a higher rate of formation of α-Guaiene and a higher final α-Guaiene to α-Bulnesene ratio. The expected change in ratio was confirmed experimentally.

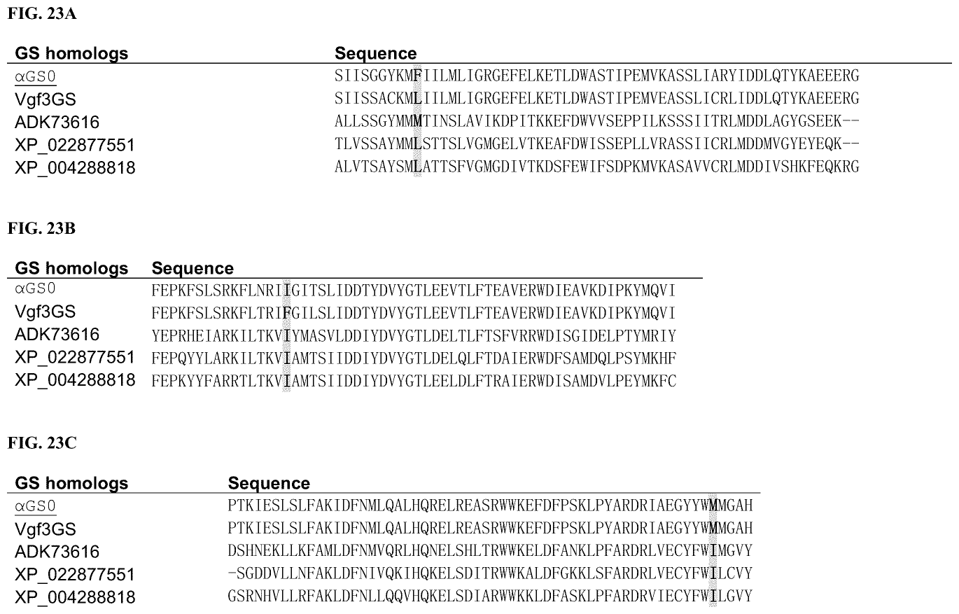

Given these results, we proposed mutations for other GS homologs to similarly shift product profile by modifying cation-π interactions (Table 14, primary mutation) or by shifting the position of the intermediates relative to nearby aromatic residues (Table 14, secondary mutation). No mutations were selected for residues aligned to αGS position 407 because sequence alignments indicate that there were no aromatic residues at the aligned positions (FIG. 23A-C).

TABLE 14
Mutation design for Alternate GS Genes

| GS Gene | Primary mutation | Secondary mutation |
|---|---|---|
| αGS0 | 1293F | M273I |
| Vgf3GS0 | — | M272I |
| ADK73616 | I306F | — |
| XP_022877551 | I301F | — |
| XP_004288818 | I306F | — |

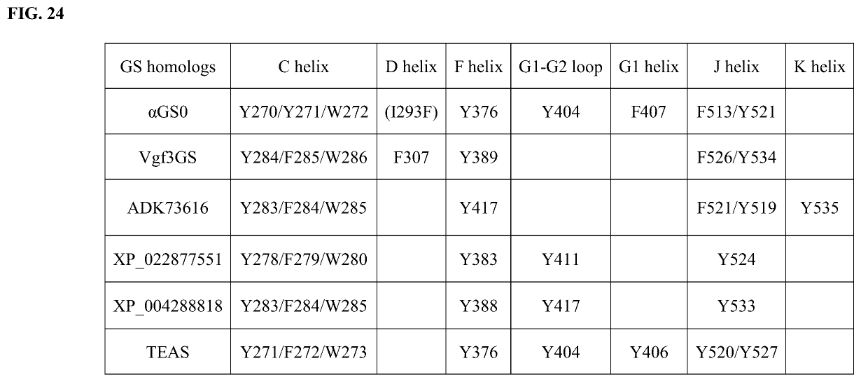

In order to discover other aromatic residues in sesquiterpene cyclases that could be mutated to modify product profile, aromatic residues in the substrate binding pockets of various sesquiterpene cyclases were identified (FIG. 24). Some positions show conservation of aromatic residues, such as a triplet of aromatic residues on the C helix. Mutating conserved positions may disrupt protein stability or catalysis. Other positions, however, vary depending on the product profile of the enzyme, such as those on the D helix. The variable positions should be good mutational targets for changing product profile by disrupting cation-π interactions with intermediates.

### Example 4: Analyses of Cytochrome P450 Reductase (CPR) and Alcohol Dehydrogenase (ADH) Homologs

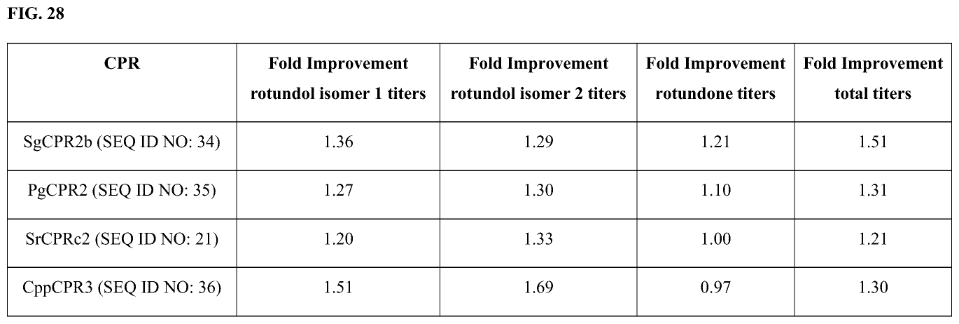

The biosynthetic pathway for the production of rotundone is illustrated in FIG. 1B. Farnesyl diphosphate is converted to α-Guaiene by an α-Guaiene Terpene Synthase (αGTPS or αGS) enzyme, and α-Guaiene is converted to (−)-rotundone by an α-Guaiene Oxidase (αGOX or αGO). The αGO enzyme requires the presence of an electron transfer protein, such as a cytochrome P450 reductase (CPR), that is capable of transferring electrons to the enzyme. In vivo production of rotundol and rotundone was analyzed in strains expressing the following CPR homologs: SgCPR2b (SEQ ID NO: 34), PgCPR2 (SEQ ID NO: 35), SrCPRc2 (SEQ ID NO: 21), and CppCPR3 (SEQ ID NO: 36). The strains co-expressed α-GS (SEQ ID NO: 28), GO (SEQ ID NO: 30), and an ADH (SEQ ID NO: 10). A strain co-expressing a CPR (SEQ ID NO: 20), α-GS (SEQ ID NO: 28), GO (SEQ ID NO: 30), and an ADH (SEQ ID NO: 10) was used as a control. Fermentation was performed in a 96 well plate for 72 hours. Fold improvements in the titres of rotundol isomers and rotundone and total oxygenated species were calculated based in comparison with the strain expressing SEQ ID NO: 20. As shown in FIG. 28, the CPRs provided an improvement in the production of rotundol isomer 1, rotundol isomer 2, rotundone and/or total oxygenated species.

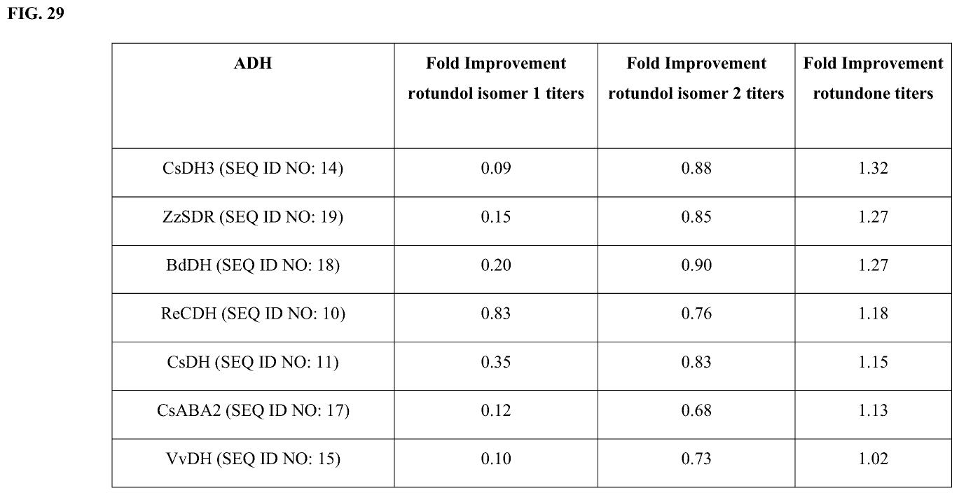

The αGO oxidizes at least a portion of the α-Guaiene to alcohol intermediates (e.g., (2R)-rotundol or (2S)-rotundol). These are to be converted to rotundone by αGO and an alcohol dehydrogenase (ADH). Thus, the microbial host cell expressing the following alcohol dehydrogenases (ADH) were evaluated: CsDH3 (SEQ ID NO: 14), ZzSDR (SEQ ID NO: 19), BdDH (SEQ ID NO: 18), ReCDH (SEQ ID NO: 10), CsDH (SEQ ID NO: 11), CSABA2 (SEQ ID NO: 17), and VvDH (SEQ ID NO: 15). In vivo production of rotundol and rotundone containing these ADH homologs from a strain co-expressing α-GS (SEQ ID NO: 28), GO (SEQ ID NO: 30), and an CPR (SEQ ID NO:20). A strain co-expressing a CPR (SEQ ID NO: 20), α-GS (SEQ ID NO: 28), GO (SEQ ID NO: 30), and an ADH (SEQ ID NO: 10) was used as a control. Fermentation was performed in a 96 well plate for 72 hours. Fold improvements in the titres of rotundol isomers and rotundone and total oxygenated species were calculated based in comparison with the strain expressing SEQ ID NO: 10. As shown in FIG. 29, the ADH enzymes provided an improvement in the production of rotundone, with concomitant decrease in rotundol isomer 1 and/or rotundol isomer 2.

SEQUENCES
α-Guaiene Synthase
αGS0 (SEQ ID NO: 1)
MASSAKLGSASEDVSRRDANYHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDENMLQALHQRELREASRWWKEFDFPSKLPYARDRIAEGYYWMMGAHFEPKFSLSRKELNRI
IGITSLIDDTYDVYGTLEEVTLFTEAVERWDIEAVKDIPKYMQVIYTGMLGIFEDFKDNLINARGKDYCIDYA
IEVFKEIVRSYQREAEYFHTGYVPSYDEYMENSIISGGYKMFIILMLIGRGEFELKETLDWASTIPEMVKASS
LIARYIDDLQTYKAEEERGETVSAVRCYMREFGVSEEQACKKMREMIEIEWKRLNKTTLEADEISSSVVIPSL
NFTRVLEVMYDKGDGYSDSQGVTKDRIAALLRHAIEI
αGS1 (SEQ ID NO: 2)
MASSAKLGSASEDVSRRDANYHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDENMLQALHQRELREASRWWKEFDFPSKLPYARDRIAEGYYWMMGAHFEPKFSLSRKELNRI
IGITSLIDDTYDVYGTLEEVTLFTEAVERWDIEAVKDIPKYMQVIYTGMLGIFEDFKDNLINARGKDYCIDYA
IEVFKEIVRAYQREAEYFHTGYVPSYDEYMENSIISGGYKMLIILMLIGRGEFELKETLDWASTIPEMVKASS
LIARLIDDLQTYKAEEERGETVSAVRCYMREFGVSEEQACKKMREMIEIEWKRLNKTTLEADEISSSVVIPSL
NFTRVLEVMYDKGDGYSDSQGVTKDRIAALLRHAIEI
αGS2 (SEQ ID NO: 3)
MASSAKLGSASEDVSRRDANYHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDFNMLQALHQRELREASRWWKEFDFPSKLPYARDRIAEGYYWMMGAHFEPKFSLSRKELTRI
IGITSLIDDTYDVYGTLEEVTLFTEAVERWDIEAVKDIPKYMQVIYTGMLGIFEDFKDNLINARGKDYCIDYA
IEVFKEIVRAYQREAEYFHTGYVPSYDEYMENSIISGGYKMLIILMLIGRGEFELKETLDWASTIPEMVKASS
LIARLIDDLQTYKAEEERGETVSAVRCYMREFGVSEEQACKKMREMIEIEWKRLNKTTLEADEISSSVVIPSL
NFTRVLEVMYDKGDGYSDSQGVTKDRIAALLRHAIEI
αGS3 (SEQ ID NO: 4)
MASSAKLGSASEDVSRRDANYHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDFNMLQALHQRELREASRWWKEFDFPSKLPYARDRIAEGYYWMMGAHFEPKFSLSRKFLARI
FGITSLIDDTYDVYGTLEEVTLFTEAVERWDIEAVKDIPKYMQVIYTGMLGIFEDFKDNLINARGKDYCIDYA
IEVFKEIVRAYQREAEYFHTGYVPSYDEYMENSIISGGYKMLIILMLIGRGEFELKETLDWASTIPEMVKASS
LIARLIDDLQTYKAEEERGETVSAVRCYMREFGVSEEQACKKMREMIEIEWKRLNKTTLEADEISSSVVIPSL
NFTRVLEVMYDKGDGYSDSQGVTKDRIAALLRHAIEI
αGS4 (SEQ ID NO: 5)
MASSAKLGSASEDVSRRDANYHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDENMLQALHQRELREASRWWKEFDFPSKLPYARDRIAEGYYWIMGAHFEPKFSLSRKFLARI
FGITSLIDDTYDVYGTLEEVTLFTEAVERWDIEAVKDIPKYMQVIYTGMLGIFEDFKDNLINARGKDYCIDYA
IEVFKEIVRAYQREAEYFHTGYVPSYDEYMENSIVSGGYKMLIILMLIGRGEFELKETLDWASTIPEMVKASS
LIARLIDDVQTYKAEEERGETVSAVRCYMREFGVSEEQACKKMREMIEIEWKRLNKTTLEADEISSSVVIPSL
NFTRVLEVMYDKGDGYSDSQGVTKDRIAALLRHAIEI
αGS5 (SEQ ID NO: 28)
MASSAKLGSASEDVSRRDANYHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDENMLQALHQRELREASRWWKEFDFPSKLPYARDRIAEGYYWIMGAHFEPKFSLSRKFLARI
FGIVSLIDDTYDVYGTLEEVTLFTEAVERWDITAVKDIPKYMQVIYTGMLGIFEDFKDNLINARGKDYCIDYA
IEVFKEIVRAYQREAEYFHTGYVPSYDEYMENSIVSGGYKMLIILMLIGRGEFELKETLDWASTIPEMVKASS
LIARLIDDVQTYKAEEERGETVSAVRCYMREFGVSEEQACKKMREMIEIEWKRLNKTTLEADEISSSVVIPSL
NFTRVLEVMYDKGDGYSDSQGVTKDRIAALLRHAIEI
αGS6 (SEQ ID NO: 31)
MASSAKLGSASEDVSRRDANFHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDENMLQALHQRELREASRWWKEFDFPSKLPYARDRIAESYYWIMGAHFEPKFSLSRKFLARI
FGIVSLIDDTYDVYGTLEEVTLFTEAVERWDITAVKDIPKYMQVIYTGMLGIFEDFKDNLINARGKDYCIDYA
IEVFKEIVRAYQREAEYFHTGYVPSYDEYMENSIVSGGYKMLIILMLIGRGEFELKETLDWASTIPEMVKASS
LIARLIDDVVTYKAEEERGETVSAVRCYMREFGVSEEQACKKMREMIEIEWKRLNKTTLEADEISSSVVIPSL
NFTRVLEVMYDKGDGYSDSQGVTKDRIAALLRHPIEI
αGS7 (SEQ ID NO: 32)
MASSAKLGSASEDVSRRDANFHPTVWGDFFLTHSSNFLENNDSILEKHEELKQEVRNLLVVETSDLPSKIQLT
DEIIRLGVGYHFETEIKAQLEKLHDHQLHLNFDLLTTSVWFRLLRGHGFSISSDVFKRFKNTKGEFETEDART
LWCLYEATHLRVDGEDILEEAIQFSRKKLEALLPELSFPLNECVRDALHIPYHRNVQRLAARQYIPQYDAEPT
KIESLSLFAKIDENMLQALHQRELREASRWWKEFDFPSKLPYARDRIAESYYWIMGAHFEPKFSLSRKFLARI
FGIVSLIDDTYDVYGTLEEVTLFTEAVERWDITAVKDIPKYMQVIYTGMLGIFEDFKDNLINARGKDYCIDYA
IEVFKEIVRAYQREAEYFHTGYVPSYDEYMENSIVSGGYKMLIILMLIGRGEFELKETLDWASTIPEMVKASS
LIARLIDDVQTYKAEEERGETVSAVRCYMREFGVSEEQACKKMREMIEDEWKRLNKTTLEADEISSSVVIPSL
NFTRVLEVMYDKGDGYSDSQGVTKDRIAALLRHPIEI
Guaiene Oxidase
GO (SEQ ID NO: 6)
MAWEYALIGLVVGIIIGAVAMRWYLKSYTSARRSQSNHLPRVPEVPGVPLLGNLLQLKEKKPYMTFTKWAATY
GPIYSIKTGATSVVVVSSNEIAKEALVTRFQSISTRNLSKALKVLTADKQMVAMSDYDDYHKTVKRHILTAVL
GPNAQKKHRIHRDIMMDNISTQLHEFVKNNPEQEEVDLRKIFQSELFGLAMRQALGKDVESLYVEDLKITMNR
DEILQVLVVDPMMGAIDVDWRDFFPYLKWVPNKKFENTIQQMYIRREAVMKSLIKEQKKRIASGEKLNSYIDY
LLSEAQTLTDQQLLMSLWEPIIESSDTTMVTTEWAMYELAKNPKLQDRLYRDIKSVCGSEKITEEHLSQLPYI
TAIFHETLRKHSPVPILPLRHVHEDTVLGGYHVPAGTELAVNIYGCNMDKNVWENPEEWNPERFMKENETIDF
QKTMAFGGGKRVCAGSLQALLIASIGIGRMVQEFEWKLKDMTQEEVNTIGLTNQMLRPLRAIIKPRI
GO1 (SEQ ID NO: 7)
MAQDLRLILIIVGAIAIIALLVHGFWYLKSYTSARRSQSNHLPRVPEVPGVPLLGNLLQLKEKKPYMTFTKLA
ATYGPIYSIKTGATSVVVVSSNEIAKEALVTRFQSISTRNLPKALKVLTADKQMVAMSDYDDYHKTVKRHILT
AVLGPNAQKKHRIHRDIMMDNVSTQLHAFVKNNPEQEEVDLRKIFQSELFGLAMRQALGKDVESLYVEDLKIT
MNRDEILQVLVVDPMRGAISVDWRDFFPYLKWIPNKKFDNTIQQMYIRREAVMKSLIKEQKKRIASGEKLNSY
IDYLLSEAQTLTDQQLLMSLWEPIILSSDTTMVTTEWAMYELAKNPKLQERLYQDIKSVCGSEKITEEHLSQL
PYLTAIFHETLRKHSPVPILPLRHVHEDTVLGGYHVPAGTELAVNIYGCNMDKNVWENPEEWNPERFMKENET
IDFQKTMAFGGGRRVCAGSLQALLIASIGIGRMVQEFEWKLKDMTQEEVDTIGLTNHMAKPLRAIIKPRI
GO2 (SEQ ID NO: 8)
MAQDLRLILIIVGAIAIIALLVHGFWYLKSYTSARRSQSNHLPRVPEVPGVPLLGNLLQLKEKKPYMTFTKLA
ATYGPIYSIKTGATSVVVVSSNEIAKEALVTRFQSISTRNLPKALKVLTADKQMVAMSDYDDYHKTVKRHILT
AVLGPNAQKKHRIHRDIMMDNVSTQLHAFVKNNPEQEEVDLRKIFQSELFGLAMRQALGKDVESLYVEDLKIT
MNRDEILQVLVVDPMRGAASVDWRDFFPYLKWIPNKKFDNTIQQMYIRREAVMKSLIKEQKKRIASGEKLNSY
IDYLLSEAQTLTDQQLLMSLWEPIILSTDTTMVTTEWAMYELAKNPKLQERLYQDIKSVCGSEKITEEHLSQL
PYLTAIFHETLRKHSPVPILPLRHVHEDTVLGGYHVPAGTELAVNIYGCNMDKNVWENPEEWNPERFMKENET
IDFQKTMAFGGGRRVCAGSLQALLIASIGIGRMVQEFEWKLKDMTQEEVDTIGLTNHMAKPLRAIIKPRI
GO3 (SEQ ID NO: 9)
MAQDLRLILIIVGAIAIIALLVHGFWYLKSYTSARRSQSNHLPRVPEVPGVPLLGNLLQLKEKKPYMTFTKLA
ATYGPIYSIKTGATSVVVVSSNEIAKEALVTRFQSISTRNLPKALKVLTADKQMVAMSDYDDYHKTVKRHILT
AVLGPNAQKKHRIHRDIMMDNVSTQLHAFVKNNPEQEEVDLRKIFQSELFGLAMRQALGKDVESLYVEDLKIT
MNRDEILQVLVVDPMRGAASVDWRDFFPYLKWIPNKKFDNTIQQMYIRREAVMKSLIKEQKKRIASGEKLNSY
IDYLLSEAQTLTDQQLLMSLWEPIIASSDTTMVTTEWAMYELAKNPKLQERLYQDIKSVCGSEKITEEHLSQL
PYLTATFHETLRKHSPVPILPLRHVHEDTVLGGYHVPAGTELAVNIYGCNMDKNVWENPEEWNPERFMKENET
IDFQKTMAFGGGRRVCAGSLQALLIASIGIGRMVQEFEWKLKDMTQEEVDTGGLTNHMAKPLRAIIKPRI
GO4 (SEQ ID NO: 29)
MAQDLRLILIIVGAIAIIALLVHGFWYLKSYTSARRSQSNHLPRVPEVPGVPLLGNLLQLKEKKPYMTFTKLA
ATYGPIYSIKTGATSVVVVSSNEIAKEALVTRFQSISTRNLPKALKVLTADKQMVAMSDYDDYHKTVKRHILT
AVLGPNAQKKHRIHRDIMMDNVSTQLHAFVKNNPEQEEVDLRKIFQSELFGLAMRQALGKDVESLYVEDLKIT
MNRDEILQVLVVDPMRGAASVDWRDFFPYLKWIPNKKFDNTIQQMYIRREAVMKSLIKEQKKRIASGEKLNSY
IDYLLSEAQTLTDQQLLMSLWEPIIASSDTTMVTTEWAMYELAKNPKLQERLYQDIKSVCGSEKITEEHLSQL
PYLTATFHETLRKHSPVPILPLRHVHEDTVLGGYHVPAGTELAVNIYGCNMDKNVWENPEEWNPERFMKENET
IDFQKTMAFGGGRRVCAGSLQALLIASIGIGRMVQEFEWKLKDMTQEEVDQGGLTNSMAKPLRAIIKPRI
GO5 (SEQ ID NO: 30)
MAQDLRLILIIVGAIAIIALLVHGFWYLKSYTSARRSQSNHLPRVPEVPGVPLLGNLLQLKEKKPYMTFTKLA
ATYGPIYSIKTGATSVVVVSSNEIAKEALVTRFQSISTRNLPKALKVLTADKQMVAMSDYDDYHKTVKRHILT
AVLGPNAQKKHRIHRDIMMDNVSTQLHAFVKNNPEQEEVDLRKIFQSELFGLAMRQALGKDVESLYVEDLKIT
MNRDEILQVLVVDPMRGAASVDWRDFFPYLKWIPNKKFDNTIQQMYIRREAVMKSLIKEQKKRIASGEKLNSY
IDYLLSEAQTLTDQQLLMSLWEPIIASSDTTMVTTEWAMYELAKNPKLQERLYQDIKSVCGSEKITEEHLSQL
PYLTATFHETLRKHSPVPILPLRHVHEDTVLGGYHVPAGTELAVNIYGCNMDKNVWENPEEWNPERFMKENET
INFQKTMAFGGGRRVCAGSLQALLIASIGIGRMVQEFEWKLKDMNQEEVDQGGLTNSMAKPLRAIIKPRI
GO6 (SEQ ID NO: 33)
MAQDLRLILIIVGAIAIIALLVHGFWYLKSYTSARRSQSNHLPRVPEVPGVPLLGNLLQLKEKKPYMTFTKLA
ATYGPIYSIKTGATSVVVVSSNEIAKEALVTRFQSISTRNLPKALKVLTADKQMVAMSDYDDYHKTVKRHILT
AVLGPNAQKKHRIHRDIMMDNVSTQLHAFVKNNPEQEAVDLRKIFQSELFGLAMRQALGKDVESLYVEDLKIT
MNRDEILQVLVVDPMRGAASVDWRDFFPYLKWIPNKKFDNTIQQMYIRREAVMKSLIKEQKKRIASGEKLNSY
IDYLLSEAQTLTDQQLLMSLWEPIIASSDTTMVTTEWAMYELAKNPKLQERLYQDIKSVCGSEKITEEHLSQL
PYLTATFHETLRKHSPVPILPLRYVHEDTVLGGYHVPAGTELAVNIYGCNMDKNVWENPEEWNPEREMKENET
INFQKTMAFGGGRRVCAGSLQALLIASIGIGRMVQEFEWKLKDMNQEEVDQGGLTNSMAKPLHAIIKPRI
Alcohol Dehydrogenase
Rhodococcus erythropolis CDH (SEQ ID NO: 10)
MARVEGQVALITGAARGQGRSHAIKLAEEGADVILVDVPNDVVDIGYPLGTADELDQTAKDVENLGRKAIVIH
ADVRDLESLTAEVDRAVSTLGRLDIVSANAGIASVPFLSHDIPDNTWRQMIDINLTGVWHTAKVAVPHILAGE
RGGSIVLTSSAAGLKGYAQISHYSAAKHGVVGLMRSLALELAPHRVRVNSLHPTQVNTPMIQNEGTYRIFSPD
LENPTREDFEIASTTTNALPIPWVESVDVSNALLFLVSEDARYITGAAIPVDAGTTLK
CsDH [Citrus sinensus] (SEQ ID NO: 11)
MATPPISSLISQRLLGKVALVTGGASGIGEGIVRLFHRHGAKVCFVDVQDELGYRLQESLVGDKDSNIFYSHC
DVTVEDDVRRAVDLTVTKFGTLDIMVNNAGISGTPSSDIRNVDVSEFEKVFDINVKGVEMGMKYAASVMIPRK
QGSIISLGSVGSVIGGIGPHHYISSKHAVVGLTRSIAAELGQHGIRVNCVSPYAVPTNLAVAHLPEDERTEDM
FTGFREFAKKNANLQGVELTVEDVANAVLFLASEDARYISGDNLIVDGGFTRVNHSERVER
CsDH1 [Citrus sinensus] (SEQ ID NO: 12)
MSKPRLQGKVAIIMGAASGIGEATAKLFAEHGAFVIIADIQDELGNQVVSSIGPEKASYRHCDVRDEKQVEET
VAYAIEKYGSLDIMYSNAGVAGPVGTILDLDMAQFDRTIATNLAGSVMAVKYAARVMVANKIRGSIICTTSTA
STVGGSGPHAYTISKHGLLGLVRSAASELGKHGIRVNCVSPFGVATPFSAGTINDVEGFVCKVANLKGIVLKA
KHVAEAALFLASDESAYVSGHDLVVDGGFTAVTNVMSMLEGHG
CsDH2 [Citrus sinensus] (SEQ ID NO: 13)
MSNPRMEGKVALITGAASGIGEAAVRLFAEHGAFVVAADVQDELGHQVAASVGTDQVCYHHCDVRDEKQVEET
VRYTLEKYGKLDVLFSNAGIMGPLTGILELDLTGFGNTMATNVCGVAATIKHAARAMVDKNIRGSIICTTSVA
SSLGGTAPHAYTTSKHALVGLVRTACSELGAYGIRVNCISPFGVATPLSCTAYNLRPDEVEANSCALANLKGI
VLKAKHIAEAALFLASDESAYISGHNLAVDGGFTVVNHSSSSAT
CsDH3 [Citrus sinensus] (SEQ ID NO: 14)
MTTAGSRDSPLVAQRLLGKVALVTGGATGIGESIVRLFHKHGAKVCVVDINDDLGQHLCQTLGPTTRFIHGDV
AIEDDVSRAVDFTVANFGTLDIMVNNAGMGGPPCPDIREFPISTFEKVFDINTKGTFIGMKHAARVMIPSKKG
SIVSISSVTSAIGGAGPHAYTASKHAVLGLTKSVAAELGQHGIRVNCVSPYAILTNLALAHLHEDERTDDARA
GFRAFIGKNANLQGVDLVEDDVANAVLFLASDDARYISGDNLFVDGGFTCTNHSLRVFR
VvDH [Vitis vinifera] (SEQ ID NO: 15)
MAATSIDNSPLPSQRLLGKVALVTGGATGIGESIVRLFLKQGAKVCIVDVQDDLGQKLCDTLGGDPNVSFFHC
DVTIEDDVCHAVDFTVTKFGTLDIMVNNAGMAGPPCSDIRNVEVSMFEKVFDVNVKGVFLGMKHAARIMIPLK
KGTIISLCSVSSAIAGVGPHAYTGSKCAVAGLTQSVAAEMGGHGIRVNCISPYAIATGLALAHLPEDERTEDA
MAGFRAFVGKNANLQGVELTVDDVAHAAVFLASDEARYISGLNLMLDGGFSCTNHSLRVER
VvDH1 [Vitis vinifera] (SEQ ID NO: 16)
MSTASSGDVSLLSQRLVGKVALITGGATGIGESIARLFYRHGAKVCIVDIQDNPGQNLCRELGTDDACFFHCD
VSIEIDVIRAVDFVVNRFGKLDIMVNNAGIADPPCPDIRNTDLSIFEKVFDVNVKGTFQCMKHAARVMVPQKK
GSIISLTSVASVIGGAGPHAYTGSKHAVLGLTKSVAAELGLHGIRVNCVSPYAVPTGMPLAHLPESEKTEDAM
MGMRAFVGRNANLQGIELTVDDVANSVVFLASDEARYVSGLNLMLDGGFSCVNHSLRVER
CsABA2 [Citrus sinensus] (SEQ ID NO: 17)
MSNSNSTDSSPAVQRLVGRVALITGGATGIGESTVRLFHKHGAKVCIADVQDNLGQQVCQSLGGEPDTFFCHC
DVTKEEDVCSAVDLTVEKFGTLDIMVNNAGISGAPCPDIREADLSEFEKVFDINVKGVFHGMKHAARIMIPQT
KGTIISICSVAGAIGGLGPHAYTGSKHAVLGLNKNVAAELGKYGIRVNCVSPYAVATGLALAHLPEEERTEDA
MVGFRNFVARNANMQGTELTANDVANAVLFLASDEARYISGTNLMVDGGFTSVNHSLRVER
BdDH [Brachypodium distachyon] (SEQ ID NO: 18)
MSAAAAVSSSSSPRLEGKVALVTGGASGIGEAIVRLFRQHGAKVCIADVQDEAGQQVRDSLGDDAGTDVLFVH
CDVTVEEDVSRAVDAAAEKFGTLDIMVNNAGITGDKVTDIRNLDFAEVRKVFDINVHGMLLGMKHAARVMIPG
KKGSIVSLASVASVMGGMGPHAYTASKHAVVGLTKSVALELGKHGIRVNCVSPYAVPTALSMPHLPQGEHKGD
AVRDFLAFVGGEANLKGVDLLPKDVAQAVLYLASDEARYISALNLVVDGGFTSVNPNLKAFED
ZzSDR [Zingiber zerumbet] (SEQ ID NO: 19)
MRLEGKVALVTGGASGIGESIARLFIEHGAKICIVDVQDELGQQVSQRLGGDPHACYFHCDVTVEDDVRRAVD
FTAEKYGTIDIMVNNAGITGDKVIDIRDADENEFKKVFDINVNGVFLGMKHAARIMIPKMKGSIVSLASVSSV
IAGAGPHGYTGAKHAVVGLTKSVAAELGRHGIRVNCVSPYAVPTRLSMPYLPESEMQEDALRGELTFVRSNAN
LKGVDLMPNDVAEAVLYLATEESKYVSGLNLVIDGGFSIANHTLQVFE
Cytochrome P450 Reductases
Camtotheca acuminta CPR (SEQ ID NO: 20)
MAQSSSVKVSTFDLMSAILRGRSMDQTNVSFESGESPALAMLIENRELVMILTTSVAVLIGCFVVLLWRRSSG
KSGKVTEPPKPLMVKTEPEPEVDDGKKKVSIFYGTQTGTAEGFAKALAEEAKVRYEKASFKVIDLDDYAADDE
EYEEKLKKETLTFFFLATYGDGEPTDNAARFYKWFMEGKERGDWLKNLHYGVFGLGNRQYEHENRIAKVVDDT
IAEQGGKRLIPVGLGDDDQCIEDDFAAWRELLWPELDQLLQDEDGTTVATPYTAAVLEYRVVFHDSPDASLLD
KSFSKSNGHAVHDAQHPCRANVAVRRELHTPASDRSCTHLEFDISGTGLVYETGDHVGVYCENLIEVVEEAEM
LLGLSPDTFFSIHTDKEDGTPLSGSSLPPPFPPCTLRRALTQYADLLSSPKKSSLLALAAHCSDPSEADRLRH
LASPSGKDEYAQWVVASQRSLLEVMAEFPSAKPPIGAFFAGVAPRLQPRYYSISSSPRMAPSRIHVTCALVFE
KTPVGRIHKGVCSTWMKNAVPLDESRDCSWAPIFVRQSNFKLPADTKVPVLMIGPGTGLAPFRGFLQERLALK
EAGAELGPAILFFGCRNRQMDYIYEDELNNFVETGALSELIVAFSREGPKKEYVQHKMMEKASDIWNMISQEG
YIYVCGDAKGMARDVHRTLHTIVQEQGSLDSSKTESMVKNLQMNGRYLRDVW
Stevia rebaudiana CPR (SEQ ID NO: 21)
MAQSDSVKVSPFDLVSAAMNGKAMEKLNASESEDPTTLPALKMLVENRELLTLFTTSFAVLIGCLVELMWRRS
SSKKLVQDPVPQVIVVKKKEKESEVDDGKKKVSIFYGTQTGTAEGFAKALVEEAKVRYEKTSFKVIDLDDYAA
DDDEYEEKLKKESLAFFFLATYGDGEPTDNAANFYKWFTEGDDKGEWLKKLQYGVFGLGNRQYEHENKIAIVV
DDKLTEMGAKRLVPVGLGDDDQCIEDDFTAWKELVWPELDQLLRDEDDTSVTTPYTAAVLEYRVVYHDKPADS
YAEDQTHTNGHVVHDAQHPSRSNVAFKKELHTSQSDRSCTHLEFDISHTGLSYETGDHVGVYSENLSEVVDEA
LKLLGLSPDTYFSVHADKEDGTPIGGASLPPPFPPCTLRDALTRYADVLSSPKKVALLALAAHASDPSEADRL
KFLASPAGKDEYAQWIVANQRSLLEVMQSFPSAKPPLGVFFAAVAPRLQPRYYSISSSPKMSPNRIHVTCALV
YETTPAGRIHRGLCSTWMKNAVPLTESPDCSQASIFVRTSNFRLPVDPKVPVIMIGPGTGLAPFRGFLQERLA
LKESGTELGSSIFFFGCRNRKVDFIYEDELNNFVETGALSELIVAFSREGTAKEYVQHKMSQKASDIWKLLSE
GAYLYVCGDAKGMAKDVHRTLHTIVQEQGSLDSSKAELYVKNLQMSGRYLRDVW
Arabidopsis thaliana CPR1 (SEQ ID NO: 22)
MATSALYASDLFKQLKSIMGTDSLSDDVVLVIATTSLALVAGFVVLLWKKTTADRSGELKPLMIPKSLMAKDE
DDDLDLGSGKTRVSIFFGTQTGTAEGFAKALSEEIKARYEKAAVKVIDLDDYAADDDQYEEKLKKETLAFFCV
ATYGDGEPTDNAARFYKWFTEENERDIKLQQLAYGVFALGNRQYEHENKIGIVLDEELCKKGAKRLIEVGLGD
DDQSIEDDFNAWKESLWSELDKLLKDEDDKSVATPYTAVIPEYRVVTHDPRFTTQKSMESNVANGNTTIDIHH
PCRVDVAVQKELHTHESDRSCIHLEFDISRTGITYETGDHVGVYAENHVEIVEEAGKLLGHSLDLVFSIHADK
EDGSPLESAVPPPFPGPCTLGTGLARYADLLNPPRKSALVALAAYATEPSEAEKLKHLTSPDGKDEYSQWIVA
SQRSLLEVMAAFPSAKPPLGVFFAAIAPRLQPRYYSISSSPRLAPSRVHVTSALVYGPTPTGRIHKGVCSTWM
KNAVPAEKSHECSGAPIFIRASNFKLPSNPSTPIVMVGPGTGLAPFRGFLQERMALKEDGEELGSSLLFFGCR
NRQMDFIYEDELNNFVDQGVISELIMAFSREGAQKEYVQHKMMEKAAQVWDLIKEEGYLYVCGDAKGMARDVH
RTLHTIVQEQEGVSSSEAEAIVKKLQTEGRYLRDVW
A. thaliana CPR2 (SEQ ID NO: 23)
MASSSSSSSTSMIDLMAAIIKGEPVIVSDPANASAYESVAAELSSMLIENRQFAMIVTTSIAVLIGCIVMLVW
RRSGSGNSKRVEPLKPLVIKPREEEIDDGRKKVTIFFGTQTGTAEGFAKALGEEAKARYEKTRFKIVDLDDYA
ADDDEYEEKLKKEDVAFFFLATYGDGEPTDNAARFYKWFTEGNDRGEWLKNLKYGVFGLGNRQYEHENKVAKV
VDDILVEQGAQRLVQVGLGDDDQCIEDDFTAWREALWPELDTILREEGDTAVATPYTAAVLEYRVSIHDSEDA
KENDINMANGNGYTVFDAQHPYKANVAVKRELHTPESDRSCIHLEFDIAGSGLTYETGDHVGVLCDNLSETVD
EALRLLDMSPDTYFSLHAEKEDGTPISSSLPPPFPPCNLRTALTRYACLLSSPKKSALVALAAHASDPTEAER
LKHLASPAGKDEYSKWVVESQRSLLEVMAEFPSAKPPLGVFFAGVAPRLQPRFYSISSSPKIAETRIHVTCAL
VYEKMPTGRIHKGVCSTWMKNAVPYEKSENCSSAPIFVRQSNFKLPSDSKVPIIMIGPGTGLAPFRGFLQERL
ALVESGVELGPSVLFFGCRNRRMDFIYEEELQRFVESGALAELSVAFSREGPTKEYVQHKMMDKASDIWNMIS
QGAYLYVCGDAKGMARDVHRSLHTIAQEQGSMDSTKAEGFVKNLQTSGRYLRDVW
A. thaliana eATR2 (SEQ ID NO: 24)
MASSSSSSSTSMIDLMAAIIKGEPVIVSDPANASAYESVAAELSSMLIENRQFAMIVTTSIAVLIGCIVMLVW
RRSGSGNSKRVEPLKPLVIKPREEEIDDGRKKVTIFFGTQTGTAEGFAKALGEEAKARYEKTRFKIVDLDDYA
ADDDEYEEKLKKEDVAFFFLATYGDGEPTDNAARFYKWFTEGNDRGEWLKNLKYGVFGLGNRQYEHENKVAKV
VDDILVEQGAQRLVQVGLGDDDQCIEDDFTAWREALWPELDTILREEGDTAVATPYTAAVLEYRVSIHDSEDA
KFNDITLANGNGYTVFDAQHPYKANVAVKRELHTPESDRSCIHLEFDIAGSGLTMKLGDHVGVLCDNLSETVD
EALRLLDMSPDTYFSLHAEKEDGTPISSSLPPPFPPCNLRTALTRYACLLSSPKKSALVALAAHASDPTEAER
LKHLASPAGKDEYSKWVVESQRSLLEVMAEFPSAKPPLGVFFAGVAPRLQPRFYSISSSPKIAETRIHVTCAL
VYEKMPTGRIHKGVCSTWMKNAVPYEKSEKLFLGRPIFVRQSNFKLPSDSKVPIIMIGPGTGLAPFRGELQER
LALVESGVELGPSVLFFGCRNRRMDFIYEEELQRFVESGALAELSVAFSREGPTKEYVQHKMMDKASDIWNMI
SQGAYLYVCGDAKGMARDVHRSLHTIAQEQGSMDSTKAEGFVKNLQTSGRYLRDVW
S. rebaudiana CPR3 (SEQ ID NO: 25)
MAQSNSVKISPLDLVTALESGKVLDTSNASESGESAMLPTIAMIMENRELLMILTTSVAVLIGCVVVLVWRRS
STKKSALEPPVIVVPKRVQEEEVDDGKKKVTVFFGTQTGTAEGFAKALVEEAKARYEKAVFKVIDLDDYAADD
DEYEEKLKKESLAFFFLATYGDGEPTDNAARFYKWFTEGDAKGEWLNKLQYGVFGLGNRQYEHENKIAKVVDD
GLVEQGAKRLVPVGLGDDDQCIEDDFTAWKELVWPELDQLLRDEDDTTVATPYTAAVAEYRVVFHEKPDALSE
DYSYTNGHAVHDAQHPCRSNVAVKKELHSPESDRSCTHLEFDISNTGLSYETGDHVGVYCENLSEVVNDAERL
VGLPPDTYFSIHTDSEDGSPLGGASLPPPFPPCTLRKALTCYADVLSSPKKSALLALAAHATDPSEADRLKEL
ASPAGKDEYSQWIVASQRSLLEVMEAFPSAKPSLGVFFASVAPRLQPRYYSISSSPKMAPDRIHVTCALVYEK
TPAGRIHKGVCSTWMKNAVPMTESQDCSWAPIYVRTSNFRLPSDPKVPVIMIGPGTGLAPFRGFLQERLALKE
AGTDLGLSILFFGCRNRKVDFIYENELNNFVETGALSELIVAFSREGPTKEYVQHKMSEKASDIWNLLSEGAY
LYVCGDAKGMAKDVHRTLHTIVQEQGSLDSSKAELYVKNLQMSGRYLRDVW
Artemisia annua CPR (SEQ ID NO: 26)
MAQSTTSVKLSPFDLMTALLNGKVSFDTSNTSDTNIPLAVEMENRELLMILTTSVAVLIGCVVVLVWRRSSSA
AKKAAESPVIVVPKKVTEDEVDDGRKKVTVFFGTQTGTAEGFAKALVEEAKARYEKAVFKVIDLDDYAAEDDE
YEEKLKKESLAFFFLATYGDGEPTDNAARFYKWFTEGEEKGEWLDKLQYAVFGLGNRQYEHENKIAKVVDEKL
VEQGAKRLVPVGMGDDDQCIEDDFTAWKELVWPELDQLLRDEDDTSVATPYTAAVAEYRVVFHDKPETYDQDQ
LTNGHAVHDAQHPCRSNVAVKKELHSPLSDRSCTHLEFDISNTGLSYETGDHVGVYVENLSEVVDEAEKLIGL
PPHTYFSVHADNEDGTPLGGASLPPPFPPCTLRKALASYADVLSSPKKSALLALAAHATDSTEADRLKFLASP
AGKDEYAQWIVASHRSLLEVMEAFPSAKPPLGVFFASVAPRLQPRYYSISSSPRFAPNRIHVTCALVYEQTPS
GRVHKGVCSTWMKNAVPMTESQDCSWAPIYVRTSNFRLPSDPKVPVIMIGPGTGLAPFRGFLQERLAQKEAGT
ELGTAILFFGCRNRKVDFIYEDELNNFVETGALSELVTAFSREGATKEYVQHKMTQKASDIWNLLSEGAYLYV
CGDAKGMAKDVHRTLHTIVQEQGSLDSSKAELYVKNLQMAGRYLRDVW
Pelargonium graveolens CPR (SEQ ID NO: 27)
MAQSSSGSMSPFDEMTAIIKGKMEPSNASLGAAGEVTAMILDNRELVMILTTSIAVLIGCVVVFIWRRSSSQT
PTAVQPLKPLLAKETESEVDDGKQKVTIFFGTQTGTAEGFAKALADEAKARYDKVTFKVVDLDDYAADDEEYE
EKLKKETLAFFFLATYGDGEPTDNAARFYKWFLEGKERGEWLQNLKFGVFGLGNRQYEHFNKIAIVVDEILAE
QGGKRLISVGLGDDDQCIEDDFTAWRESLWPELDQLLRDEDDTTVSTPYTAAVLEYRVVFHDPADAPTLEKSY
SNANGHSVVDAQHPLRANVAVRRELHTPASDRSCTHLEFDISGTGIAYETGDHVGVYCENLAETVEEALELLG
LSPDTYFSVHADKEDGTPLSGSSLPPPFPPCTLRTALTLHADLLSSPKKSALLALAAHASDPTEADRLRHLAS
PAGKDEYAQWIVASQRSLLEVMAEFPSAKPPLGVFFASVAPRLQPRYYSISSSPRIAPSRIHVTCALVYEKTP
TGRVHKGVCSTWMKNSVPSEKSDECSWAPIFVRQSNFKLPADAKVPIIMIGPGTGLAPFRGFLQERLALKEAG
TELGPSILFFGCRNSKMDYIYEDELDNFVQNGALSELVLAFSREGPTKEYVQHKMMEKASDIWNLISQGAYLY
VCGDAKGMARDVHRTLHTIAQEQGSLDSSKAESMVKNLQMSGRYLRDVW
SgCPR2b (SEQ ID NO: 34)
MAQSESRSMKVSPLELMSAIIRKAMDPSRESSESVREVATLILENREFVMILTTLLAVLIGCVVVLVWKRSSG
QKAKPFEPPKQLIVKEPEPEVDDGKKKVTVFFGTQTGTAEGFAKALAEEAKARYEKATERVVDLDDYAADDDE
YEEKLKKETLAIFFLATYGDGEPTDNAARFYKWFSEGKEKGDWISNLQYAVFGLGNRQYEHFNKIAKVVDEQL
AEQGGKRLVPVGLGDDDQCIEDDFSAWREALWPELDKLLRDDDDSTTVATPYTAAVLEYRVVFYDAADVSVED
KRWAFANGHAVYDAQHPCRANVAMRKELHTPASDRSCIHLEFDISGTGLTYETGDHVGVFCENLDETVEDAIR
LIGLSPETYFSIHTDKDDGTPLGGSSLPPPFAPCTLRTALTQYADLLSSPKKSALVALAAHASDPAEADRLRH
LSSPAGKDEYAQWIIASQRSLLEVMAEFPSAKPPLGVFFAAVAPRLQPRYYSISSSPRMAPSRIHVTCALVYD
KTPTGRIHKGVCSTWMKNAVPLEESQACSWAPIYVRQSNFKLPTDSKLPIIMIGPGTGLAPFRGFLQERLALK
EAGVELGHSILFFGCRNRKMDYIYEDELSNFAETGALSELIVAFSREGPTKEYVQHKMVDKASDIWNILSQGG
YIYVCGDAKGMARDVHRTLHNIVQEQGSLDSSKAESMVKNLQMSGRYLRDVW
PgCPR2 (SEQ ID NO: 35)
MAESLNGGSIDLSIPASMALLFENRELLMLLTTSIAILIGCVVVLVWRRSSSQGSAKSFEPPKLTISKIEPEE
EVDDGKKKVTIFFGTQTGTAEGFAKAFAEEAKARYEKAKFKVIDLDDYAEDDDEYEAKLKKESLALFFLATYG
DGEPTDNAARFYKWFSEGEEKDEWLKNLQYGVFGLGNRQYEHFNKIAKVVDDGLAEQGAKRLVPVGMGDDDQC
IEDDFTAWRELAWPELDQLLLDKEDAAVATPYTAAVLEYRVVVHDQTDTSLLDRNLSTLNGHTVYDAQHPCRS
NVAVKRELHTPASDRSCIHLEFDISHTGLSYETGDHVGVYCENLIEIVEEAERLLGIAPATYFSVHTDKEDGT
PLSGGSLPPPFPPCTLRTALTRYADLLSSPKKSALLALAAHASDSSEADRLRFLASPAGKDEYAQWLVANQRS
LLEVMAEFPSAKPPLGVFFASIAPRLQPRYYSISSSPRMAPSRIHVTCALVYEKTPTGRIHKGVCSTWMKNAV
SLEENNDCSWAPIFVRQSNFKLPSDTKVPIIMIGPGTGLAPFRGFLQERLALKEAGAELGPAVLYFGCRNRKL
DFIYEDELNNFVETGVISELVLAFSREGATKEYVQHKMSQKALEVWNLISQGAYIYVCGDAKGMARDVHRMLH
TIAQEQGALDSSKAESLVKNLQMTGRYLRDVW
CppCPR3 (SEQ ID NO: 36)
MAQSESRSMKVSTLELISAIIRKAMDPSQDSSESVKEVATLMMENREFVMIVTTSIAVLIGCVVVLVWKRSSS
QKVKSFEPPKQLIVKEPEPEVEDGKKKVTVFFGTQTGTAEGFAKALAEEAKARYEKATERVVDLDDYAADDDE
YEEKLRKETITIFFLATYGDGEPTDNAARFYKWFSEGKEKGEWISNLQYAVFGLGNRQYEHENKIALVVDEQL
AEQGGKRLVPVGLGDDDQCIEDDFTAWREALWPELDKLLRDEDDSTTASTPYTAAVLEYRVVFYDAADVPGGD
KRWSLANGHSVYDAQHPCRSNVAVRKELHTPASDRSCTHLEFDISGTGLTYETGDHVGVFCENLDEVVEEALR
LLGLSPETYFSIHADKEDGTPLTGSSLPPLFAPCTLRTALTQYADLLSSPKKSALVALAAHASDPAEADRLRH
LSSPAGKDEYSQWIIASQRSLLEVMAEFPSARPPLGVFFAAVAPRLQPRYYSISSSPRMAPSRINVTCALVYD
KTPTGRIHKGVCSTWMKSAVSLEESQACSWAPIYVRQSNFKLPTDSKLPIIMIGPGTGLAPFRGFLQERLALK
EAGVELAHSILFFGCRNRNMDYIYEYELNNFVETGALSELIVAFSREGPSKEYVQHKMVEKASEIWNLLSQGA
YIYVCGDAKGMARDVHRTLHNIVQEQGSLDSSKAESMVKNLQMSGRYLRDVW

1. A method for producing rotundone, comprising:
providing a host cell producing farnesyl diphosphate, and expressing a heterologous rotundone biosynthesis pathway, the rotundone biosynthesis pathway comprising an α-Guaiene Synthase (αGS) and an α-Guaiene Oxidase (αGO), wherein:
the αGS comprises an amino acid sequence having at least 70% sequence identity to amino acids 258 to 548 of SEQ ID NO: 1 and having one or more amino acid modifications that increase αGS biosynthesis as compared to SEQ ID NO: 1; and/or
the αGO comprises an amino acid sequence having at least 70% sequence identity to the amino acid sequence of SEQ ID NO: 6 and having one or more amino acid modifications that increase rotundone biosynthesis as compared to SEQ ID NO: 6;
culturing the host cell under conditions to allow for rotundone production; and
recovering rotundone from the culture.

2. The method of claim 1, wherein the αGS comprises an amino acid sequence having at least 80% sequence identity to amino acids 258 to 548 of SEQ ID NO: 1.

3. The method of claim 1, wherein the αGS comprises an amino acid sequence having at least 85% sequence identity to amino acids 258 to 548 of SEQ ID NO: 1.

4. The method of claim 1, wherein the αGS comprises an amino acid sequence having at least 90% sequence identity to amino acids 258 to 548 of SEQ ID NO: 1.

5. The method of claim 1, wherein the αGS comprises an amino acid sequence having at least 95% sequence identity to amino acids 258 to 548 of SEQ ID NO: 1.

6. The method of claim 1, wherein the αGS comprises an amino acid sequence having at least 97% sequence identity to amino acids 258 to 548 of SEQ ID NO: 1.

7. The method of any one of claims 1 to 6, wherein the αGS comprises one or more amino acid substitutions with respect to SEQ ID NO: 1 within positions 258 to 548.

8. The method of claim 7, wherein the αGS comprises from 2 to 20 or from 2 to 10 amino acid substitutions with respect to SEQ ID NO: 1 within positions 269 to 500.

9. The method of claim 8, wherein the αGS comprises one or more substitutions in a secondary structure element selected from the G2, D, J, and C helices.

10. The method of claim 9, wherein at least one substitution of the αGS is on the D helix, and wherein the substitution on the D helix adds an aromatic residue, which is optionally phenylalanine.

11. The method of claim 10, wherein at least one substitution of the αGS is on the G2 helix, and wherein the substitution on the G2 helix is optionally a removal of an aromatic residue.

12. The method of any one of claims 1 to 11, wherein one or more amino acid modifications are made to the αGS with respect to SEQ ID NO: 6 that stabilize a carbocation at C2 or C6 of the cyclized intermediate, and/or to destabilize a carbocation at C7 of the cyclized intermediate.

13. The method of claim 12, wherein the one or more amino acid modifications to the αGS stabilize the carbocation at C2 or C6 by adding a cation-π interaction between an aromatic side chain and a carbocation at C2 or C6; and/or destabilize the carbocation at C7 by removing an interaction between an aromatic or aliphatic side chain and a carbocation at C7.

14. The method of any one of claims 7 to 13, wherein the αGS comprises one or more substitutions at positions selected from 290, 325, 407, 499, 495, 341, 273, 375, 443, 447, 294, 269, 440, 21, 448, and 545 with respect to SEQ ID NO: 1.

15. The method of claim 14, wherein the αGS comprises at least two, at least three, or at least four amino acid substitutions with respect to SEQ ID NO: 1 at positions selected from 290, 325, 407, 499, 495, 341, 273, 375, 443, 447, 294, 269, 21, 448, and 545.

16. The method of any one of claims 1 to 15, wherein the αGS comprises one or more substitutions with respect to SEQ ID NO: 1 selected from S375A, F407L, and Y443L.

17. The method of claim 16, wherein the αGS comprises the amino acid sequence of SEQ ID NO: 2.

18. The method of claim 1, wherein the αGS comprises the amino acid sequence of SEQ ID NO: 2, optionally with from 1 to 20 or from 1 to 10 or from 1 to 5, or from 1 to 3 amino acid modifications independently selected from substitutions, deletions, and insertions.

19. The method of claim 18, wherein the αGS comprises one or more amino acid modifications with respect to SEQ ID NO: 2 that are selected from Table 1, and optionally comprises the substitution N290T.

20. The method of claim 19, wherein the αGS comprises from 2 to 20 or from 2 to 10 amino acid substitutions with respect to SEQ ID NO: 2 within positions 258 to 548.

21. The method of claim 20, wherein the αGS comprises one or more amino acid substitutions with respect to SEQ ID NO: 2 at positions selected from 290, 325, 499, 495, 341, 273, 447, 294, 439, 504, 369, and 206 of SEQ ID NO: 2.

22. The method of claim 20, wherein the αGS comprises the amino acid sequence of SEQ ID NO: 3, or comprises the amino acid sequence of amino acids 258 to 548 of SEQ ID NO: 3.

23. The method of claim 1, wherein the αGS comprises the amino acid sequence of SEQ ID NO: 3, optionally with from 1 to 20 or from 1 to 10 or from 1 to 5, or from 1 to 3 amino acid modifications independently selected from substitutions, deletions, and insertions.

24. The method of claim 23, wherein the αGS comprises one or more amino acid modifications with respect to SEQ ID NO: 3 that are selected from Table 2.

25. The method of claim 24, wherein the αGS comprises the substitution T290A and/or I293F with respect to SEQ ID NO: 3.

26. The method of claim 25, wherein the αGS comprises the amino acid sequence of SEQ ID NO: 4.

27. The method of claim 1, wherein the αGS comprises the amino acid sequence of SEQ ID NO: 4, optionally with from 1 to 20 or from 1 to 10, or from 1 to 5, or from 1 to 3 amino acid modifications independently selected from substitutions, deletions, and insertions.

28. The method of claim 27, wherein the αGS comprises from 2 to 20 or from 2 to 10 amino acid substitutions with respect to SEQ ID NO: 4 within positions 258 to 548.

29. The method of claim 27 or 28, wherein the αGS comprises one or more amino acid substitutions with respect to SEQ ID NO: 4 at positions selected from 447, 372, 296, 400, 293, 439, 452, 292, 480, 203, 369, 325, 173, 189, 220, 513, 516, 440, 290, 481, 149, 212, 399, 172, and 273 of SEQ ID NO: 4.

30. The method of claim 28 or 29, wherein the αGS comprises the substitutions L447V, I400V, and M273I, with respect to SEQ ID NO: 4.

31. The method of claim 30, wherein the αGS comprises the amino acid sequence of SEQ ID NO: 5, or comprises the amino acid sequence of amino acids 258 to 548 of SEQ ID NO: 5.

32. The method of claim 1, wherein the αGS comprises the amino acid sequence of SEQ ID NO: 5, optionally with from 1 to 20 or from 1 to 10, or from 1 to 5, or from 1 to 3 amino acid modifications independently selected from substitutions, deletions, and insertions.

33. The method of claim 32, wherein the αGS comprises from 2 to 20 or from 2 to 10, or from 2 to 5 amino acid modifications with respect to SEQ ID NO: 5 within positions 258 to 548 of SEQ ID NO: 5.

34. The method of claim 32 or 33, wherein the αGS comprises one or more amino acid modifications listed in Table 4.

35. The method of claim 34, wherein the αGS comprises at least the modifications T296V and E325T with respect to SEQ ID NO: 5.

36. The method of claim 35, wherein the αGS comprises the amino acid sequence of SEQ ID NO: 28, or comprises the amino acid sequence of amino acids 258 to 548 of SEQ ID NO: 28.

37. The method of claim 1, wherein the αGS comprises amino acid substitutions at one or more positions selected from 273, 290, 293, 296, 325, 375, 400, 407, 443, and 447, with respect to SEQ ID NO: 1.

38. The method of claim 37, wherein the αGS comprises one or more amino acid substitutions selected from M273I, N290T, N290A, I293F, T296V, E325T, S375A, I400L, I400V, F407L, Y443L, Y443V, Y443F, and L447V with respect to SEQ ID NO: 1.

39. The method of claim 1, wherein the αGS enzyme comprises an amino acid sequence that has at least about 90% sequence identity, or at least about 95% sequence identity, or at least about 97% sequence identity, or at least 98% sequence identity, or at least 99% sequence identity with amino acids 258 to 548 of SEQ ID NO: 28, wherein the α-Guaiene Synthase comprises a phenylalanine at the position corresponding to position 293 of SEQ ID NO: 28, and optionally retains a non-aromatic residue at the position corresponding to position 407 of SEQ ID NO: 28.

40. The method of claim 39, wherein the αGS comprises one or more of:
an Ile, Leu, or Val at the position corresponding to position 273 of SEQ ID NO: 28;
an Ala, Gly, Thr, or Ser at the position corresponding to position 290 of SEQ ID NO: 28;
a Val, Leu, Ile, or Ala at the position corresponding to position 296 of SEQ ID NO: 28;
a Thr or Ser at the position corresponding to position 325 of SEQ ID NO: 28;
an Ala, Gly, or Leu at the position corresponding to position 375 of SEQ ID NO: 28;
a Val or Leu at the position corresponding to position 400 of SEQ ID NO: 28;
a Leu, Val, or Ile at the position corresponding to position 407 of SEQ ID NO: 28;
a Leu, Val, or Ile at the position corresponding to position 443 of SEQ ID NO: 28; and
a Val at the position corresponding to position 447 of SEQ ID NO: 28.

41. The method of claim 1, wherein the αGS comprises the amino acid sequence of SEQ ID NO: 28, optionally with from 1 to 20 or from 1 to 10, or from 1 to 5, or from 1 to 3 amino acid modifications independently selected from substitutions, deletions, and insertions.

42. The method of claim 41, wherein the αGS comprises from 2 to 20 or from 2 to 10, or from 2 to 5 amino acid modifications with respect to SEQ ID NO: 28 within positions 258 to 548 of SEQ ID NO: 28.

43. The method of claim 41 or 42, wherein the αGS comprises one or more amino acid modifications listed in Table 5.

44. The method of claim 43, wherein αGS comprises one, two, three, four, or five modifications selected from G269S, Y21F, Q448V, and A545P with respect to SEQ ID NO: 28.

45. The method of claim 44, wherein the αGS comprises the amino acid sequence of SEQ ID NO: 31, or comprises the amino acid sequence of amino acids 258 to 548 of SEQ ID NO: 31.

46. The method of claim 1, wherein the αGS comprises amino acid substitutions at one or more positions selected from 21, 269, 273, 290, 293, 296, 325, 375, 400, 407, 443, 447, 448 and 545 with respect to SEQ ID NO: 1.

47. The method of claim 46, wherein the αGS comprises one or more amino acid substitutions selected from Y21F, G269S, M273I, N290T, N290A, I293F, T296V, E325T, S375A, I400L, I400V, F407L, Y443L, Y443V, Y443F, L447V, Q448V, and A545P with respect to SEQ ID NO: 1.

48. The method of claim 1, wherein the αGS enzyme comprises an amino acid sequence that has at least about 90% sequence identity, or at least about 95% sequence identity, or at least about 97% sequence identity, or at least 98% sequence identity, or at least 99% sequence identity with amino acids 258 to 548 of SEQ ID NO: 31 or 32, wherein the α-Guaiene Synthase comprises a phenylalanine at the position corresponding to position 293 of SEQ ID NO: 31 or 32, and optionally retains a non-aromatic residue at the position corresponding to position 407 of SEQ ID NO: 31 or 32.

49. The method of claim 1, wherein the αGS comprises the amino acid sequence of SEQ ID NO: 31, optionally with from 1 to 20 or from 1 to 10, or from 1 to 5, or from 1 to 3 amino acid modifications independently selected from substitutions, deletions, and insertions.

50. The method of claim 49, wherein the αGS comprises from 2 to 20 or from 2 to 10, or from 2 to 5 amino acid modifications with respect to SEQ ID NO: 31 within positions 258 to 548 of SEQ ID NO: 31.

51. The method of claim 49 or 50, wherein the αGS comprises one or more amino acid modifications listed in Table 6.

52. The method of claim 51, wherein the αGS comprises at least the modifications V448Q and I487D with respect to SEQ ID NO: 31.

53. The method of claim 52, wherein the αGS comprises the amino acid sequence of SEQ ID NO: 32, or comprises the amino acid sequence of amino acids 258 to 548 of SEQ ID NO: 32.

54. The method of any one of claims 1 to 53, wherein the αGO comprises an amino acid sequence having at least 80% sequence identity to the amino acid sequence of SEQ ID NO: 6.

55. The method of claim 54, wherein the αGO comprises an amino acid sequence having at least 85% sequence identity to the amino acid sequence of SEQ ID NO: 6.

56. The method of claim 54, wherein the αGO comprises an amino acid sequence having at least 90% sequence identity to the amino acid sequence of SEQ ID NO: 6.

57. The method of claim 54, wherein the αGO comprises an amino acid sequence having at least 95% sequence identity to the amino acid sequence of SEQ ID NO: 6.

58. The method of claim 54, wherein the αGO comprises an amino acid sequence having at least 97% sequence identity to the amino acid sequence of SEQ ID NO: 6.

59. The method of any one of claims 54 to 58, wherein the αGO comprises a substitution at one or more positions relative to SEQ ID NO: 6 selected from: 497, 235, 451, 72, 490, 496, 368, 318, 387, and 386.

60. The method of claim 59, wherein the αGO comprises one or more substitutions selected from Table 6.

61. The method of claim 60, wherein the αGO comprises amino acid substitution(s) selected from M235R and E318L with respect to SEQ ID NO: 6.

62. The method of claim 61, wherein the αGO comprises the amino acid sequence of SEQ ID NO: 7, optionally with from 1 to 10 or from 1 to 5 amino acid modifications independently selected from substitutions, insertions, and deletions.

63. The method of claim 62, wherein the αGO comprises one or more substitutions selected from Table 7 relative to SEQ ID NO: 7.

64. The method of claim 63, wherein the αGO comprises an amino acid substitution selected from I238A and/or S320T with respect to SEQ ID NO: 7.

65. The method of claim 64, wherein the αGO comprises the amino acid sequence of SEQ ID NO: 8.

66. The method of claim 54, wherein the αGO comprises the amino acid sequence of SEQ ID NO: 8, optionally with from 1 to 10 or from 1 to 5 amino acid modifications independently selected from substitutions, insertions, and deletions.

67. The method of claim 66, wherein the αGO comprises one or more amino acid modifications listed in Table 8 with respect to SEQ ID NO: 8.

68. The method of claim 67, wherein the αGO comprises one or more amino acid substitutions selected from L318A, T320S, and I490G with respect to SEQ ID NO: 8.

69. The method of claim 68, wherein the αGO comprises the amino acid sequence of SEQ ID NO: 9.

70. The method of claim 54, wherein the αGO comprises the amino acid sequence of SEQ ID NO: 9, optionally having from 1 to 10 or from 1 to 5 amino acid modifications independently selected from substitutions, insertions, and deletions.

71. The method of claim 70, wherein the αGO comprises one or more amino acid modifications with respect to SEQ ID NO: 9 selected from Table 9.

72. The method of claim 71, wherein the αGO comprises amino acid substitution(s) selected from T489Q and H495S with respect to SEQ ID NO: 9.

73. The method of claim 72, wherein the αGO comprises the amino acid sequence of SEQ ID NO: 29.

74. The method of claim 54, wherein the αGO comprises the amino acid sequence of SEQ ID NO: 29, optionally having from 1 to 10 or from 1 to 5 amino acid modifications independently selected from substitutions, insertions, and deletions.

75. The method of claim 74, wherein the αGO comprises one or more amino acid modifications with respect to SEQ ID NO: 29 selected from Table 10.

76. The method of claim 75, wherein the αGO comprises the substitution D440G with respect to SEQ ID NO: 29.

77. The method of claim 54, wherein the αGO comprises the amino acid sequence of SEQ ID NO: 30.

78. The method of claim 54, wherein the αGO comprises the amino acid sequence of SEQ ID NO: 30, optionally having from 1 to 10 or from 1 to 5 amino acid modifications independently selected from substitutions, insertions, and deletions.

79. The method of claim 78, wherein the αGO comprises one or more amino acid modifications with respect to SEQ ID NO: 30 selected from Table 11.

80. The method of claim 79, wherein the αGO comprises the substitution E184A, H389Y and R501H with respect to SEQ ID NO: 30.

81. The method of claim 54, wherein the αGO comprises the amino acid sequence of SEQ ID NO: 33.

82. The method of claim 54, wherein the αGO comprises the amino acid sequence of SEQ ID NO: 33, optionally having from 1 to 10 or from 1 to 5 amino acid modifications independently selected from substitutions, insertions, and deletions.

83. The method of claim 82, wherein the αGO comprises one or more amino acid modifications with respect to SEQ ID NO: 33 selected from Table 12.

84. The method of claim 54, wherein the αGO enzyme comprises an amino acid sequence that has at least about 90% sequence identity to SEQ ID NO: 30, wherein the αGO comprises at least two of:
an Ala or Gly at the position corresponding to position 184 of SEQ ID NO: 30;
an Arg, Lys, Ser, or Thr at the position corresponding to position 235 of SEQ ID NO: 30;
an Ala, Leu, Thr, or Gly at the position corresponding to position 238 of SEQ ID NO: 30;
Ala or Gly at the position corresponding to position 318 of SEQ ID NO: 30;
a Gly, Ala, or Ser at the position corresponding to position 490 of SEQ ID NO: 30;
a Phe, Tyr, Trp at the position corresponding to position 389 of SEQ ID NO: 30;
a Gln, Lys, Asn, Met, Ser, Glu at the position corresponding to position 489 of SEQ ID NO: 30;
a Ser, Asn, or Thr at the position corresponding to position 495 of SEQ ID NO: 30;
a Gly, Ala, or Asn at the position corresponding to position 440 of SEQ ID NO: 30; and
a His at the position corresponding to position 501 of SEQ ID NO: 30.

85. The method of any one of claims 1 to 84, wherein the host cell expresses a heterologous cytochrome P450 reductase, optionally comprising an amino acid sequence having 70% sequence identity to SEQ ID NO: 20, SEQ ID NO: 34, SEQ ID NO: 35, SEQ ID NO: 21, or SEQ ID NO: 36.

86. The method of claim 85, wherein the cytochrome P450 reductase comprises an amino acid sequence that is at least 80%, or at least 85%, at least 90%, or at least 95%, at least 97% sequence identity with the amino acid sequence of SEQ ID NO: 20.

87. The method of claim 85, wherein the cytochrome P450 reductase comprises an amino acid sequence that is at least 80%, or at least 85%, at least 90%, or at least 95%, at least 97% sequence identity with the amino acid sequence of SEQ ID NO: 34.

88. The method of claim 85, wherein the cytochrome P450 reductase comprises an amino acid sequence that is at least 80%, or at least 85%, at least 90%, or at least 95%, at least 97% sequence identity with the amino acid sequence of SEQ ID NO: 35.

89. The method of claim 85, wherein the cytochrome P450 reductase comprises an amino acid sequence that is identical to the amino acid sequence of SEQ ID NO: 34.

90. The method of claim 85, wherein the cytochrome P450 reductase comprises an amino acid sequence that is at least 80%, or at least 85%, at least 90%, or at least 95%, at least 97% sequence identity with the amino acid sequence of SEQ ID NO: 21.

91. The method of claim 85, wherein the cytochrome P450 reductase comprises an amino acid sequence that is at least 80%, or at least 85%, at least 90%, or at least 95%, at least 97% sequence identity with the amino acid sequence of SEQ ID NO: 36.

92. The method of any one of claims 1 to 91, wherein the heterologous biosynthesis pathway further comprises an alcohol dehydrogenase.

93. The method of claim 92, wherein the alcohol dehydrogenase comprises an amino acid sequence that has at least about 70% sequence identity with SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19.

94. The method of claim 93, wherein the alcohol dehydrogenase comprises an amino acid sequence that is at least 70% sequence identity to SEQ ID NO: 10.

95. The method of claim 94, wherein the alcohol dehydrogenase comprises an amino acid sequence that is at least 80%, or at least 85%, at least 90%, or at least 95%, at least 97% sequence identity with the amino acid sequence of SEQ ID NO: 11.

96. The method of claim 94, wherein the alcohol dehydrogenase comprises an amino acid sequence that is at least 80%, or at least 85%, at least 90%, or at least 95%, at least 97% sequence identity with the amino acid sequence of SEQ ID NO: 14.

97. The method of claim 94, wherein the alcohol dehydrogenase comprises an amino acid sequence that is at least 80%, or at least 85%, at least 90%, or at least 95%, at least 97% sequence identity with the amino acid sequence of SEQ ID NO: 15.

98. The method of claim 94, wherein the alcohol dehydrogenase comprises an amino acid sequence that is at least 80%, or at least 85%, at least 90%, or at least 95%, at least 97% sequence identity with the amino acid sequence of SEQ ID NO: 17.

99. The method of claim 94, wherein the alcohol dehydrogenase comprises an amino acid sequence that is at least 80%, or at least 85%, at least 90%, or at least 95%, at least 97% sequence identity with the amino acid sequence of SEQ ID NO: 18.

100. The method of claim 94, wherein the alcohol dehydrogenase comprises an amino acid sequence that is at least 80%, or at least 85%, at least 90%, or at least 95%, at least 97% sequence identity with the amino acid sequence of SEQ ID NO: 19.

101. The method of any one of claims 1 to 100, wherein the microbial host cell further expresses a heterologous farnesyl diphosphate synthase (FPPS).

102. The method of any one of claims 1 to 101, wherein one or more enzymes of the heterologous biosynthesis pathway are expressed from extrachromosomal elements.

103. The method of any one of claims 1 to 102, wherein one or more enzymes of the heterologous biosynthesis pathway are expressed from genes that are chromosomally integrated.

104. The method of any one of claims 1 to 103, wherein the host cell is a microbial host cell overexpressing one or more enzymes in the methylerythritol phosphate (MEP) or the mevalonic acid (MVA) pathway.

105. The method of claim 104, wherein the microbial cell is a bacterium, optionally selected from Escherichia spp., Bacillus spp., Corynebacterium spp., Rhodobacter spp., Zymomonas spp., Vibrio spp., and Pseudomonas spp.

106. The method of claim 105, wherein the bacterial host cell is selected from Escherichia coli, Bacillus subtilis, Corynebacterium glutamicum, Rhodobacter capsulatus, Rhodobacter sphaeroides, Zymomonas mobilis, Vibrio natriegens, and Pseudomonas putida.

107. The method of claim 104, wherein the microbial host cell is a yeast, optionally selected from Saccharomyces, Pichia, and Yarrowia.

108. The method of claim 107, wherein the microbial host cell is Saccharomyces cerevisiae, Pichia pastoris, or Yarrowia lipolytica.

109. The method of any one of claims 104 to 108, wherein the host cell is cultured in a carbon source comprising glucose, sucrose, fructose, xylose, and/or glycerol.

110. The method of any one of claims 104 to 109, wherein culture conditions are selected from aerobic, microaerobic, and anaerobic.

111. The method of claim 110, wherein the microbial host cell is cultured at a temperature in the range of about 22° C. to about 37° C., or about 27° C. to about 37° C., or about 30° C. to about 37° C.

112. A host cell producing rotundone, comprising: an upstream biosynthesis pathway producing farnesyl diphosphate (FPP) and a heterologous rotundone biosynthesis pathway, the rotundone biosynthesis pathway comprising an α-Guaiene Synthase (αGS) and a Guaiene Oxidase (GO), wherein:
the αGS comprises an amino acid sequence having at least 70% sequence identity to the amino acid sequence of SEQ ID NO: 1 and having one or more amino acid modifications that increase αGS biosynthesis as compared to SEQ ID NO: 1; and/or
the αGO comprises an amino acid sequence having at least 70% sequence identity to the amino acid sequence of SEQ ID NO: 6 and having one or more amino acid modifications that increase rotundone biosynthesis as compared to SEQ ID NO: 6.

113. The host cell of claim 112, wherein the αGS comprises an amino acid sequence having at least 80% sequence identity to amino acids 258 to 548 of SEQ ID NO: 1.

114. The host cell of claim 112, wherein the αGS comprises an amino acid sequence having at least 85% sequence identity to amino acids 258 to 548 of SEQ ID NO: 1.

115. The host cell of claim 112, wherein the αGS comprises an amino acid sequence having at least 90% sequence identity to amino acids 258 to 548 of SEQ ID NO: 1.

116. The host cell of claim 112, wherein the αGS comprises an amino acid sequence having at least 95% sequence identity to amino acids 258 to 548 of SEQ ID NO: 1.

117. The host cell of claim 112, wherein the αGS comprises an amino acid sequence having at least 97% sequence identity to amino acids 258 to 548 of SEQ ID NO: 1.

118. The host cell of any one of claims 112 to 117, wherein the αGS comprises one or more amino acid substitutions with respect to SEQ ID NO: 1 within positions 258 to 548.

119. The host cell of claim 118, wherein the αGS comprises from 2 to 20 or from 2 to 10 amino acid substitutions with respect to SEQ ID NO: 1 within positions 269 to 500.

120. The host cell of claim 119, wherein the αGS comprises one or more substitutions in a secondary structure element selected from the G2, D, J, and C helices.

121. The host cell of claim 120, wherein at least one substitution of the αGS is on the D helix, and wherein the substitution on the D helix adds an aromatic residue, which is optionally phenylalanine.

122. The host cell of claim 121, wherein at least one substitution of the αGS is on the G2 helix, and wherein the substitution on the G2 helix is optionally a removal of an aromatic residue.

123. The host cell of any one of claims 112 to 122, wherein one or more amino acid modifications are made to the αGS that stabilize a carbocation at C2 or C6 of the cyclized intermediate, and/or to destabilize a carbocation at C7 of the cyclized intermediate.

124. The host cell of claim 123, wherein the one or more amino acid modifications to the αGS stabilize the carbocation at C2 or C6 by adding a cation-π interaction between an aromatic side chain and a carbocation at C2 or C6; and/or destabilize the carbocation at C7 by removing an interaction between an aromatic or aliphatic side chain and a carbocation at C7.

125. The host cell of any one of claims 123 to 124, wherein the αGS comprises one or more substitutions at positions selected from 290, 325, 407, 499, 495, 341, 273, 375, 443, 447, 294, 269, 21, 448, and 545 with respect to SEQ ID NO: 1.

126. The host cell of claim 125, wherein the αGS comprises at least two, at least three, or at least four amino acid substitutions with respect to SEQ ID NO: 1 at positions selected from 290, 325, 407, 499, 495, 341, 273, 375, 443, 447, 294, 269, 21, 448, and 545.

127. The host cell of claim 126, wherein the αGS comprises one or more substitutions with respect to SEQ ID NO: 1 selected from S375A, F407L, and Y443L.

128. The host cell of claim 127, wherein the αGS comprises the amino acid sequence of SEQ ID NO: 2.

129. The host cell of claim 112, wherein the αGS comprises the amino acid sequence of SEQ ID NO: 2, optionally with from 1 to 20 or from 1 to 10 or from 1 to 5, or from 1 to 3 amino acid modifications independently selected from substitutions, deletions, and insertions.

130. The host cell of claim 129, wherein the αGS comprises one or more amino acid modifications with respect to SEQ ID NO: 2 that are selected from Table 1, and optionally comprises the substitution and N290T.

131. The host cell of claim 130, wherein the αGS comprises from 2 to 20 or from 2 to 10 amino acid substitutions with respect to SEQ ID NO: 2 within positions 258 to 548.

132. The host cell of claim 131, wherein the αGS comprises one or more amino acid substitutions with respect to SEQ ID NO: 2 at positions selected from 290, 325, 499, 495, 341, 273, 447, 294, 439, 504, 369, and 206 of SEQ ID NO: 2.

133. The host cell of claim 132, wherein the αGS comprises the amino acid sequence of SEQ ID NO: 3, or comprises the amino acid sequence of amino acids 258 to 548 of SEQ ID NO: 3.

134. The host cell of claim 112, wherein the αGS comprises the amino acid sequence of SEQ ID NO: 3, optionally with from 1 to 20 or from 1 to 13 or from 1 to 5, or from 1 to 3 amino acid modifications independently selected from substitutions, deletions, and insertions.

135. The host cell of claim 134, wherein the αGS comprises one or more amino acid modifications with respect to SEQ ID NO: 3 that are selected from Table 2.

136. The host cell of claim 135, wherein the αGS comprising the substitution T290A and/or I293F with respect to SEQ ID NO: 3.

137. The host cell of claim 136, wherein the αGS comprises the amino acid sequence of SEQ ID NO: 4.

138. The host cell of claim 112, wherein the αGS comprises the amino acid sequence of SEQ ID NO: 4, optionally with from 1 to 20 or from 1 to 10, or from 1 to 5, or from 1 to 3 amino acid modifications independently selected from substitutions, deletions, and insertions.

139. The host cell of claim 138, wherein the αGS comprises from 2 to 20 or from 2 to 10 amino acid substitutions with respect to SEQ ID NO: 4 within positions 258 to 548.

140. The host cell of claim 138 or 139, wherein the αGS comprises one or more amino acid substitutions with respect to SEQ ID NO: 4 at positions selected from 447, 372, 296, 400, 293, 439, 452, 292, 480, 203, 369, 325, 173, 189, 220, 513, 516, 440, 290, 481, 149, 212, 399, 172, and 273 of SEQ ID NO: 4.

141. The host cell of claim 140, wherein the αGS comprises the substitutions L447V, 1400V, and M273I, with respect to SEQ ID NO: 4.

142. The host cell of claim 141, wherein the αGS comprises the amino acid sequence of SEQ ID NO: 5, or comprises the amino acid sequence of amino acids 258 to 548 of SEQ ID NO: 5.

143. The host cell of claim 142, wherein the αGS comprises the amino acid sequence of SEQ ID NO: 5, optionally with from 1 to 20 or from 1 to 10, or from 1 to 5, or from 1 to 3 amino acid modifications independently selected from substitutions, deletions, and insertions.

144. The host cell of claim 143, wherein the αGS comprises from 2 to 20 or from 2 to 10, or from 2 to 5 amino acid modifications with respect to SEQ ID NO: 5 within positions 258 to 548 of SEQ ID NO: 5.

145. The host cell of claim 144, comprising one or more amino acid modifications listed in Table 4.

146. The host cell of claim 145, wherein the αGS comprises at least the modifications T296V and E325T with respect to SEQ ID NO: 5.

147. The host cell of claim 146, wherein the αGS comprises the amino acid sequence of SEQ ID NO: 28, or comprises the amino acid sequence of amino acids 258 to 548 of SEQ ID NO: 28.

148. The host cell of claim 147, wherein the αGS comprises amino acid substitutions at one or more positions selected from 273, 290, 293, 296, 325, 375, 400, 407, 443, and 447, with respect to SEQ ID NO: 1.

149. The host cell of claim 148, wherein the αGS comprises one or more amino acid substitutions selected from M273I, N290T, N290A, 1293F, T296V, E325T, S375A, 1400L, I400V, F407L, Y443L, Y443V, Y443F, and L447V with respect to SEQ ID NO: 1.

150. The host cell of claim 112, wherein the αGS enzyme comprises an amino acid sequence that has at least about 90% sequence identity, or at least about 95% sequence identity, or at least about 97% sequence identity, or at least 98% sequence identity, or at least 99% sequence identity with amino acids 258 to 548 of SEQ ID NO: 28, 31, or 32, wherein the α-Guaiene Synthase comprises a phenylalanine at the position corresponding to position 293 of SEQ ID NO: 28, and optionally retains a non-aromatic residue at the position corresponding to position 407 of SEQ ID NO: 28.

151. The host cell of claim 112, wherein the αGS comprises the amino acid sequence of SEQ ID NO: 28, optionally with from 1 to 20 or from 1 to 10, or from 1 to 5, or from 1 to 3 amino acid modifications independently selected from substitutions, deletions, and insertions.

152. The host cell of claim 150, wherein the αGS comprises from 2 to 20 or from 2 to 10, or from 2 to 5 amino acid modifications with respect to SEQ ID NO: 28 within positions 258 to 548 of SEQ ID NO: 28.

153. The host cell of claim 151 or 152, comprising one or more amino acid modifications listed in Table 5.

154. The host cell of claim 153, wherein the αGS comprises at least the modifications G269S, Y21F, Q448V, and A545P with respect to SEQ ID NO: 28.

155. The host cell of claim 154, wherein the αGS comprises the amino acid sequence of SEQ ID NO: 31, or comprises the amino acid sequence of amino acids 258 to 548 of SEQ ID NO: 31.

156. The host cell of claim 112, wherein the αGS comprises amino acid substitutions at one or more positions selected from 21, 269, 273, 290, 293, 296, 325, 375, 400, 407, 443, 447, 448 and 545 with respect to SEQ ID NO: 1.

157. The host cell of claim 156, wherein the αGS comprises one or more amino acid substitutions selected from Y21F, G269S, M273I, N290T, N290A, 1293F, T296V, E325T, S375A, I400L, I400V, F407L, Y443L, Y443V, Y443F, L447V, Q448V, and A545P with respect to SEQ ID NO: 1.

158. The host cell of claim 112, wherein the αGS comprises the amino acid sequence of SEQ ID NO: 31, optionally with from 1 to 20 or from 1 to 10, or from 1 to 5, or from 1 to 3 amino acid modifications independently selected from substitutions, deletions, and insertions.

159. The host cell of claim 158, wherein the αGS comprises from 2 to 20 or from 2 to 10, or from 2 to 5 amino acid modifications with respect to SEQ ID NO: 31 within positions 258 to 548 of SEQ ID NO: 28.

160. The host cell of claim 158 or 159, comprising one or more amino acid modifications listed in Table 6.

161. The host cell of claim 160, wherein the αGS comprises at least the modifications V448Q and I487D with respect to SEQ ID NO: 31.

162. The host cell of claim 161, wherein the αGS comprises the amino acid sequence of SEQ ID NO: 32, or comprises the amino acid sequence of amino acids 258 to 548 of SEQ ID NO: 32.

163. The host cell of claim 112, wherein the αGS comprises amino acid substitutions at one or more positions selected from 21, 269, 273, 290, 293, 296, 325, 375, 400, 407, 443, 447, 448, 487, and 545 with respect to SEQ ID NO: 1.

164. The host cell of claim 163, wherein the αGS comprises one or more amino acid substitutions selected from Y21F, G269S, M273I, N290T, N290A, 1293F, T296V, E325T, S375A, I400L, I400V, F407L, Y443L, Y443V, Y443F, L447V, Q448V, 1487D, and A545P with respect to SEQ ID NO: 1.

165. The host cell of any one of claims 112 to 164, wherein the αGO comprises an amino acid sequence having at least 80% sequence identity to the amino acid sequence of SEQ ID NO: 6.

166. The host cell of claim 165, wherein the αGO comprises an amino acid sequence having at least 85% sequence identity to the amino acid sequence of SEQ ID NO: 6.

167. The host cell of claim 165, wherein the αGO comprises an amino acid sequence having at least 90% sequence identity to the amino acid sequence of SEQ ID NO: 6.

168. The host cell of claim 165, wherein the αGO comprises an amino acid sequence having at least 95% sequence identity to the amino acid sequence of SEQ ID NO: 6.

169. The host cell of claim 165, wherein the αGO comprises an amino acid sequence having at least 97% sequence identity to the amino acid sequence of SEQ ID NO: 6.

170. The host cell of any one of claims 112 to 169, wherein the αGO comprises a substitution at one or more positions relative to SEQ ID NO: 6 selected from: 497, 235, 451, 72, 490, 496, 368, 318, 387, and 386.

171. The host cell of claim 170, wherein the αGO comprises one or more substitutions selected from Table 6.

172. The host cell of claim 171, wherein the αGO comprises amino acid substitution(s) selected from M235R and E318L with respect to SEQ ID NO: 6.

173. The host cell of claim 172, wherein the αGO comprises the amino acid sequence of SEQ ID NO: 7, optionally with from 1 to 10 or from 1 to 5 amino acid modifications independently selected from substitutions, insertions, and deletions.

174. The host cell of claim 173, wherein the αGO comprises one or more substitutions selected from Table 7 relative to SEQ ID NO: 7.

175. The host cell of claim 174, wherein the αGO comprises an amino acid substitution selected from I238A and/or S320T with respect to SEQ ID NO: 7.

176. The host cell of claim 175, wherein the αGO comprises the amino acid sequence of SEQ ID NO: 8.

177. The host cell of claim 112, wherein the αGO comprises the amino acid sequence of SEQ ID NO: 8, optionally with from 1 to 10 or from 1 to 5 amino acid modifications independently selected from substitutions, insertions, and deletions.

178. The host cell of claim 177, wherein the αGO comprises one or more amino acid modifications listed in Table 8 with respect to SEQ ID NO: 8.

179. The host cell of claim 178, wherein the αGO comprises one or more amino acid substitutions selected from L318A, T320S, and I490G with respect to SEQ ID NO: 8.

180. The host cell of claim 179, wherein the αGO comprises the amino acid sequence of SEQ ID NO: 9.

181. The host cell of claim 112, wherein the αGO comprises the amino acid sequence of SEQ ID NO: 9, optionally having from 1 to 10 or from 1 to 5 amino acid modifications independently selected from substitutions, insertions, and deletions.

182. The host cell of claim 181, wherein the αGO comprises one or more amino acid modifications with respect to SEQ ID NO: 9 selected from Table 9.

183. The host cell of claim 182, wherein the αGO comprises amino acid substitution(s) selected from T489Q and H495S with respect to SEQ ID NO: 9.

184. The host cell of claim 183, wherein the αGO comprises the amino acid sequence of SEQ ID NO: 29.

185. The host cell of claim 112, wherein the αGO comprises the amino acid sequence of SEQ ID NO: 29, optionally having from 1 to 10 or from 1 to 5 amino acid modifications independently selected from substitutions, insertions, and deletions.

186. The host cell of claim 185, wherein the αGO comprises one or more amino acid modifications with respect to SEQ ID NO: 29 selected from Table 10.

187. The host cell of claim 186, wherein the αGO comprises the substitution D440G with respect to SEQ ID NO: 29.

188. The host cell of claim 112, wherein the αGO comprises the amino acid sequence of SEQ ID NO: 29.

189. The host cell of claim 112, wherein the αGO enzyme comprises an amino acid sequence that has at least about 90% sequence identity to SEQ ID NO: 30, wherein the αGO comprises at least two of:
an Ala or Gly at the position corresponding to position 184 of SEQ ID NO: 30;
an Arg, Lys, Ser, or Thr at the position corresponding to position 235 of SEQ ID NO: 30;
an Ala, Leu, Thr, or Gly at the position corresponding to position 238 of SEQ ID NO: 30;
Ala or Gly at the position corresponding to position 318 of SEQ ID NO: 30;
a Gly, Ala, or Ser at the position corresponding to position 490 of SEQ ID NO: 30;
a Phe, Tyr, Trp at the position corresponding to position 389 of SEQ ID NO: 30;
a Gln, Lys, Asn, Met, Ser, Glu at the position corresponding to position 489 of SEQ ID NO: 30;
a Ser, Asn, or Thr at the position corresponding to position 495 of SEQ ID NO: 30;
a Gly, Ala, or Asn at the position corresponding to position 440 of SEQ ID NO: 30; and
a His at the position corresponding to position 501 of SEQ ID NO: 30.

190. The method of claim 112, wherein the αGO comprises the amino acid sequence of SEQ ID NO: 30, optionally having from 1 to 10 or from 1 to 5 amino acid modifications independently selected from substitutions, insertions, and deletions.

191. The method of claim 189, wherein the αGO comprises one or more amino acid modifications with respect to SEQ ID NO: 30 selected from Table 11.

192. The method of claim 191, wherein the αGO comprises the substitution E184A, H389Y and R501H with respect to SEQ ID NO: 30.

193. The method of claim 112, wherein the αGO comprises the amino acid sequence of SEQ ID NO: 33, or an amino acid sequence having at least 80%, or at least 85%, at least 90%, or at least 95%, at least 97% sequence identity thereto.

194. The method of claim 112, wherein the αGO comprises the amino acid sequence of SEQ ID NO: 33, optionally having from 1 to 10 or from 1 to 5 amino acid modifications independently selected from substitutions, insertions, and deletions.

195. The method of claim 112, wherein the αGO comprises one or more amino acid modifications with respect to SEQ ID NO: 33 selected from Table 11.

196. The host cell of any one of claims 112 to 195, wherein the host cell expresses a heterologous cytochrome P450 reductase, optionally comprising an amino acid sequence having 70% sequence identity to SEQ ID NO: 20, SEQ ID NO: 34, SEQ ID NO: 35, SEQ ID NO: 21, or SEQ ID NO: 36.

197. The host cell of claim 196, wherein the cytochrome P450 reductase comprises an amino acid sequence that is at least 80%, or at least 85%, at least 90%, or at least 95%, at least 97% sequence identity with the amino acid sequence of SEQ ID NO: 20.

198. The host cell of claim 195, wherein the cytochrome P450 reductase comprises an amino acid sequence that is at least 80%, or at least 85%, at least 90%, or at least 95%, at least 97% sequence identity with the amino acid sequence of SEQ ID NO: 34.

199. The host cell of claim 195, wherein the cytochrome P450 reductase comprises an amino acid sequence that is at least 80%, or at least 85%, at least 90%, or at least 95%, at least 97% sequence identity with the amino acid sequence of SEQ ID NO: 35.

200. The host cell of claim 195, wherein the cytochrome P450 reductase comprises an amino acid sequence that is identical to the amino acid sequence of SEQ ID NO: 34.

201. The host cell of claim 195, wherein the cytochrome P450 reductase comprises an amino acid sequence that is at least 80%, or at least 85%, at least 90%, or at least 95%, at least 97% sequence identity with the amino acid sequence of SEQ ID NO: 21.

202. The host cell of claim 195, wherein the cytochrome P450 reductase comprises an amino acid sequence that is at least 80%, or at least 85%, at least 90%, or at least 95%, at least 97% sequence identity with the amino acid sequence of SEQ ID NO: 36.

203. The host cell of any one of claims 112 to 202, wherein the heterologous biosynthesis pathway further comprises an alcohol dehydrogenase.

204. The host cell of claim 203, wherein the alcohol dehydrogenase comprises an amino acid sequence that has at least about 70% sequence identity with SEQ ID NO: 10, SEQ ID NO: 11, SEQ ID NO: 12, SEQ ID NO: 13, SEQ ID NO: 20, SEQ ID NO: 15, SEQ ID NO: 16, SEQ ID NO: 17, SEQ ID NO: 18, or SEQ ID NO: 19.

205. The host cell of claim 204, wherein the alcohol dehydrogenase comprises an amino acid sequence that is at least 70% sequence identity to SEQ ID NO: 10.

206. The host cell of claim 204, wherein the alcohol dehydrogenase comprises an amino acid sequence that is at least 80%, or at least 85%, at least 90%, or at least 95%, at least 97% sequence identity with the amino acid sequence of SEQ ID NO: 11.

207. The host cell of claim 204, wherein the alcohol dehydrogenase comprises an amino acid sequence that is at least 80%, or at least 85%, at least 90%, or at least 95%, at least 97% sequence identity with the amino acid sequence of SEQ ID NO: 14.

208. The host cell of claim 204, wherein the alcohol dehydrogenase comprises an amino acid sequence that is at least 80%, or at least 85%, at least 90%, or at least 95%, at least 97% sequence identity with the amino acid sequence of SEQ ID NO: 15.

209. The host cell of claim 204, wherein the alcohol dehydrogenase comprises an amino acid sequence that is at least 80%, or at least 85%, at least 90%, or at least 95%, at least 97% sequence identity with the amino acid sequence of SEQ ID NO: 17.

210. The host cell of claim 204, wherein the alcohol dehydrogenase comprises an amino acid sequence that is at least 80%, or at least 85%, at least 90%, or at least 95%, at least 97% sequence identity with the amino acid sequence of SEQ ID NO: 18.

211. The host cell of claim 204, wherein the alcohol dehydrogenase comprises an amino acid sequence that is at least 80%, or at least 85%, at least 90%, or at least 95%, at least 97% sequence identity with the amino acid sequence of SEQ ID NO: 19.

212. The host cell of any one of claims 112 to 211, wherein the microbial host cell further expresses a heterologous farnesyl diphosphate synthase (FPPS).

213. The host cell of any one of claims 112 to 212, wherein one or more enzymes of the heterologous biosynthesis pathway are expressed from extrachromosomal elements.

214. The host cell of any one of claims 112 to 213, wherein one or more enzymes of the heterologous biosynthesis pathway are expressed from genes that are chromosomally integrated.

215. The host cell of any one of claims 112 to 214, wherein the host cell is a microbial host cell overexpressing one or more enzymes in the methylerythritol phosphate (MEP) or the mevalonic acid (MVA) pathway.

216. The host cell of claim 215, wherein the microbial cell is a bacterium, optionally selected from Escherichia spp., Bacillus spp., Corynebacterium spp., Rhodobacter spp., Zymomonas spp., Vibrio spp., and Pseudomonas spp.

217. The host cell of claim 216, wherein the bacterial host cell is selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida.

218. The host cell of claim 215, wherein the microbial host cell is a yeast, optionally selected from Saccharomyces, Pichia, or Yarrowia.

219. The host cell of claim 218, wherein the microbial cell is Saccharomyces cerevisiae, Pichia pastoris, and Yarrowia lipolytica.

220. An α-Guaiene Synthase comprising an amino acid sequence that has at least about 90% sequence identity with amino acids 258 to 548 of SEQ ID NO: 5, wherein the α-Guaiene Synthase comprises a Phenylalanine at the position corresponding to position 293 of SEQ ID NO: 28, and optionally a non-aromatic residue at the position corresponding to position 407 of SEQ ID NO: 28.

221. The α-Guaiene Synthase of claim 220, comprising one or more of:
an Ile, Leu, or Val at the position corresponding to position 273 of SEQ ID NO: 28;
an Ala, Gly, Thr, or Ser at the position corresponding to position 290 of SEQ ID NO: 28;
a Val, Leu, Ile, or Ala at the position corresponding to position 296 of SEQ ID NO: 28;
a Thr or Ser at the position corresponding to position 325 of SEQ ID NO: 28;
an Ala, Gly, or Leu at the position corresponding to position 375 of SEQ ID NO: 28;
a Val or Leu at the position corresponding to position 400 of SEQ ID NO: 28;
a Leu, Val, or Ile at the position corresponding to position 407 of SEQ ID NO: 28;
a Leu, Val, or Ile at the position corresponding to position 443 of SEQ ID NO: 28; and
a Val at the position corresponding to position 447 of SEQ ID NO: 28.

222. The α-Guaiene Synthase of claim 220, having a phenylalanine at the position corresponding to position 293 of SEQ ID NO: 5, and a Leucine at position 407 of SEQ ID NO: 5.

223. The α-Guaiene Synthase of any one of claims 220 to 222, wherein the synthase is recombinantly expressed, and optionally purified.

224. The α-Guaiene Synthase of claim 223, wherein the synthase is expressed in a host cell the produces farnesyl diphosphate.

225. A polynucleotide encoding the α-Guaiene Synthase of any one of claims 220 to 224.

226. A host cell comprising the polynucleotide of claim 225.

227. An α-Guaiene Synthase comprising an amino acid sequence that has at least about 90% sequence identity with amino acids 258 to 548 of SEQ ID NO: 28, wherein the α-Guaiene Synthase comprises one or more modifications selected from G269S, Y21F, Q448V, and A545P with respect to SEQ ID NO: 28.

228. An α-Guaiene Synthase comprising an amino acid sequence comprising the modifications V448Q and I487D with respect to SEQ ID NO: 31.

229. The α-Guaiene Synthase of claim 228, wherein the αGS comprises the amino acid sequence of SEQ ID NO: 32, or comprises the amino acid sequence of amino acids 258 to 548 of SEQ ID NO: 32.

230. The α-Guaiene Synthase of any one of claims 227 to 229, wherein the synthase is recombinantly expressed, and optionally purified.

231. The α-Guaiene Synthase of claim 230, wherein the synthase is expressed in a host cell the produces farnesyl diphosphate.

232. A polynucleotide encoding the α-Guaiene Synthase of any one of claims 227 to 231.

233. A host cell comprising the polynucleotide of claim 232.

234. An α-Guaiene Oxidase comprising an amino acid sequence that has at least about 90% sequence identity to SEQ ID NO: 30, wherein the αGO enzyme comprises an amino acid sequence that has at least about 90% sequence identity to SEQ ID NO: 30, wherein the αGO comprises at least two of:
an Ala or Gly at the position corresponding to position 184 of SEQ ID NO: 30;
an Arg, Lys, Ser, or Thr at the position corresponding to position 235 of SEQ ID NO: 30;
an Ala, Leu, Thr, or Gly at the position corresponding to position 238 of SEQ ID NO: 30;
Ala or Gly at the position corresponding to position 318 of SEQ ID NO: 30;
a Gly, Ala, or Ser at the position corresponding to position 490 of SEQ ID NO: 30;
a Phe, Tyr, Trp at the position corresponding to position 389 of SEQ ID NO: 30;
a Gln, Lys, Asn, Met, Ser, Glu at the position corresponding to position 489 of SEQ ID NO: 30;
a Ser, Asn, or Thr at the position corresponding to position 495 of SEQ ID NO: 30;
a Gly, Ala, or Asn at the position corresponding to position 440 of SEQ ID NO: 30; and
a His at the position corresponding to position 501 of SEQ ID NO: 30.

235. The α-Guaiene Oxidase of claim 234, wherein the oxidase is co-expressed in a host cell with a heterologous cytochrome P450 reductase.

236. The α-Guaiene Oxidase of claim 234, wherein the cytochrome P450 reductase comprises an amino acid sequence that is at least 80% identical to SEQ ID NO: 20, or at least 90%, or at least 95% identical to SEQ ID NO: 20.

237. The α-Guaiene Oxidase of any one of claims 234 to 236, wherein the oxidase is co-expressed in a host cell with a heterologous alcohol dehydrogenase enzyme.

238. The α-Guaiene Oxidase of claim 237, wherein the alcohol dehydrogenase enzyme comprises an amino acid sequence that is at least 80%, or at least 90%, or at least 95% identical to SEQ ID NO: 10.

239. An α-Guaiene Oxidase comprising an amino acid sequence that has at least about 90% sequence identity to SEQ ID NO: 30, wherein the α-Guaiene Oxidase comprises at least two of:
an Ala, Leu, Thr, or Gly at the position corresponding to position 184 of SEQ ID NO: 30;
an Arg, Lys, Ser, or Thr at the position corresponding to position 235 of SEQ ID NO: 30;
an Ala, Leu, Thr, or Gly at the position corresponding to position 238 of SEQ ID NO: 30;
a Leu, Ala, or Gly at the position corresponding to position 318 of SEQ ID NO: 30;
a Gly, Ala, or Ser at the position corresponding to position 490 of SEQ ID NO: 30;
a Phe, Tyr, Trp at the position corresponding to position 389 of SEQ ID NO: 30;
a Gln, Lys, Asn, Met, Ser, Glu at the position corresponding to position 489 of SEQ ID NO: 30;
a Ser, Asn, or Thr at the position corresponding to position 495 of SEQ ID NO: 30;
a Gly, Ala, or Asn at the position corresponding to position 440 of SEQ ID NO: 30; and
a His, Lys, or Arg at the position corresponding to position 501 of SEQ ID NO: 30.

240. The α-Guaiene Oxidase of claim 239, wherein the αGO comprises the substitution E184A, H389Y and R501H with respect to SEQ ID NO: 30.

241. The α-Guaiene Oxidase of claim 240, wherein the αGO comprises the amino acid sequence of SEQ ID NO: 33.

242. The α-Guaiene Oxidase of any one of claims 239 to 241, wherein the oxidase is co-expressed in a host cell producing α-Guaiene.

243. The α-Guaiene Oxidase of claim 242, wherein the host cell further expresses an heterologous cytochrome P450 reductase and/or alcohol dehydrogenase.

244. A polynucleotide encoding the α-Guaiene Oxidase of any one of claims 239 to 243.

245. A host cell comprising the polynucleotide of claim 244.

246. A method for producing a target cyclic terpenoid, comprising:
contacting a prenyl diphosphate with a terpene synthase capable of catalyzing cyclization of the prenyl diphosphate to produce the target cyclic terpenoid and one or more non-target cyclic terpenoids through a series of cyclic carbocation intermediates, wherein the terpene synthase comprises one or more amino acid modifications of a wild type or parent terpene synthase amino acid sequence so as:
(1) to add or position an aromatic side chain to stabilize a carbocation intermediate that deprotonates to the target cyclic terpenoid; and/or
(2) to remove or shift one or more aromatic side chains to destabilize a carbocation intermediate that deprotonates to at least one non-target cyclic terpenoid.

247. The method of claim 246, wherein the target cyclic terpenoid is a sesquiterpenoid.

248. The method of claim 246, wherein the target cyclic terpenoid is a triterpenoid.

249. The method of claim 246, wherein the target cyclic terpenoid is a monoterpenoid or a diterpenoid.

250. The method of any one of claims 246 to 249, wherein (1) an aromatic side chain is added or positioned to stabilize a cation-π interaction; and/or (2) an aromatic side chain is removed or shifted to destabilize a cation-π interaction.

251. The method of claim 250, wherein a non-aromatic side chain in the wild-type enzyme is substituted with an aromatic side chain, wherein the aromatic side chain forms a cation-π interaction with the carbocation that deprotonates to the target cyclic terpenoid.

252. The method of claim 250 or 251, wherein an aromatic side chain in the wild-type enzyme is substituted with a non-aromatic side chain, wherein the aromatic side chain in the wild-type enzyme forms a π-cation interaction with the carbocation that deprotonates to a non-target cyclic terpenoid.

253. The method of claim 251 or 252, wherein the aromatic side chain is phenylalanine.

254. The method of claim 253, wherein the amino acid modifications position the center of the benzyl ring of the phenylalanine side chain within about 5 Angstroms of the carbocation that deprotonates to the target cyclic terpenoid.

255. The method of claim 254, wherein the amino acid modifications position the center of the benzyl ring of the phenylalanine side chain within about 4.5 or within about 4.0 Angstroms of the carbocation that deprotonates to the target cyclic terpenoid.

256. The method of claim 254, wherein the amino acid modifications position the center of the benzyl ring of the phenylalanine side chain from about 3.5 to about 5.0 Angstroms of the carbocation that deprotonates to the target cyclic terpenoid.

257. The method of any one of claims 254 to 256, wherein the amino acid modifications result in removal or positioning of all aromatic or aliphatic residues to a distance that is at least about 6 Angstroms from the carbocation that deprotonates to a non-target terpenoid.

258. The method of any one of claims 246 to 257, wherein one or more amino acid modifications are made to secondary structure elements selected from the G2 helices, the D helices, the J helices, and the C helices.

259. The method of claim 258, wherein a non-aromatic residue in the G2 helices, the D helices, the J helices, or the C helices is substituted with an aromatic residue, which is optionally phenylalanine, to thereby stabilize the carbocation that protonates to the target cyclic terpenoid.

260. The method of claim 258 or 259, wherein an aromatic or aliphatic residue in the G2 helices, the D helices, the J helices, or the C helices that stabilizes a carbocation that deprotonates to a non-target helices is substituted with a non-aromatic or non-aliphatic residue.

261. The method of any one of claims 246 to 260, wherein the terpene synthase is expressed in a host cell that produces the prenyl diphosphate.

262. The method of claim 261, wherein the terpene synthase is co-expressed in the host cell with an oxidase enzyme that oxygenates the target cyclic terpenoid.

263. The method of any one of claims 246 to 262, further comprising, recovering the target cyclic terpenoid from the reaction or culture.

264. A method for making a terpene synthase enzyme, comprising:
providing a terpene synthase amino acid sequence, the terpene synthase capable of catalyzing cyclization of a prenyl diphosphate to produce a target cyclic terpenoid and one or more non-target cyclic terpenoids through a series of cyclic carbocation intermediates,
making one or more amino acid modifications to the terpene synthase amino acid sequence so as:
(1) to add or position an aromatic side chain to stabilize a carbocation intermediate that deprotonates to the target cyclic terpenoid; and/or
(2) to remove or shift one or more aromatic side chains to destabilize a carbocation intermediate that deprotonates to at least one non-target cyclic terpenoid; and
recombinantly producing the terpene synthase enzyme.

265. The method of claim 264, wherein the target cyclic terpenoid is a sesquiterpenoid.

266. The method of claim 264, wherein the target cyclic terpenoid is a triterpenoid.

267. The method of claim 264, wherein the target cyclic terpenoid is a monoterpenoid or a diterpenoid.

268. The method of any one of claims 264 to 267, wherein (1) an aromatic side chain is added or positioned to add or stabilize a cation-π interaction; and/or (2) an aromatic side chain is removed or shifted to destabilize a cation-π interaction.

269. The method of claim 265, wherein a non-aromatic side chain is substituted with an aromatic side chain, wherein the aromatic side chain forms a cation-π interaction with the carbocation that deprotonates to the target cyclic terpenoid.

270. The method of claim 268 or 269, wherein an aromatic side chain in the terpene synthase is substituted with a non-aromatic side chain, wherein the aromatic side chain in the terpene synthase enzyme forms a cation-π interaction with the carbocation that deprotonates to a non-target cyclic terpenoid.

271. The method of claim 270, wherein the aromatic side chain is phenylalanine.

272. The method of claim 271, wherein the amino acid modifications position the center of the benzyl ring of a phenylalanine side chain within about 5 Angstroms of the carbocation that deprotonates to the target cyclic terpenoid.

273. The method of claim 271, wherein the amino acid modifications position the center of the benzyl ring of a phenylalanine side chain within about 4.5 or within about 4.0 Angstroms of the carbocation that deprotonates to the target cyclic terpenoid.

274. The method of claim 271, wherein the amino acid modifications position the center of the benzyl ring of a phenylalanine side chain from about 3.5 to about 5.0 Angstroms of the carbocation that deprotonates to the target cyclic terpenoid.

275. The method of any one of claims 264 to 274, wherein the amino acid modifications result in removal or positioning of all aromatic or aliphatic residues to a distance that is at least about 6 Angstroms from the carbocation that deprotonates to a non-target terpenoid.

276. The method of any one of claims 264 to 275, wherein one or more amino acid modifications are made to secondary structure elements selected from the G2 helices, the D helices, the J helices, and the C helices.

277. The method of claim 276, wherein a non-aromatic residue in the G2 helices, the D helices, the J helices, or the C helices is substituted with an aromatic residue, which is optionally phenylalanine, to thereby stabilize the carbocation that protonates to the target cyclic terpenoid.

278. The method of claim 276 or 277, wherein an aromatic or aliphatic residue in the G2 helices, the D helices, the J helices, or the C helices that stabilizes a carbocation that deprotonates to a non-target helices is substituted with a non-aromatic or non-aliphatic residue.

279. The method of any one of claims 264 to 278, wherein amino acid modifications are guided by a structural model of the terpene synthase.

280. The method of claim 279, wherein the structural model is a homology model, the homology model optionally based on structural coordinates for 5-epi-aristolochene synthase.

281. The method of any one of claims 264 to 280, wherein the terpene synthase is expressed in a host cell that produces the prenyl diphosphate.

282. The method of claim 281, wherein the terpene synthase is co-expressed in the host cell with an oxidase enzyme that oxygenates the target cyclic terpenoid.

283. A method for making a target terpenoid compound, comprising, contacting the enzyme made according to the method of any one of claims 264 to 282 with a prenyl diphosphate substrate, and recovering the target terpenoid compound.


SEQUENCE LISTING (36 sequences)

SEQ ID NO: 1 - protein, 548 aa, Artificial Sequence
  Synthetic Sequence
MASSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAEGYYWMMGAHFEPKFSLSRKFLNRIIGITSLIDDTYDVYGTLEEVTLFTEAVERWDIEAVKDIPKYMQVIYTGMLGIFEDFKDNLINARGKDYCIDYAIEVFKEIVRSYQREAEYFHTGYVPSYDEYMENSIISGGYKMFIILMLIGRGEFELKETLDWASTIPEMVKASSLIARYIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 2 - protein, 548 aa, Artificial Sequence
  Synthetic Sequence
MASSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAEGYYWMMGAHFEPKFSLSRKFLNRIIGITSLIDDTYDVYGTLEEVTLFTEAVERWDIEAVKDIPKYMQVIYTGMLGIFEDFKDNLINARGKDYCIDYAIEVFKEIVRAYQREAEYFHTGYVPSYDEYMENSIISGGYKMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 3 - protein, 548 aa, Artificial Sequence
  Synthetic Sequence
MASSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAEGYYWMMGAHFEPKFSLSRKFLTRIIGITSLIDDTYDVYGTLEEVTLFTEAVERWDIEAVKDIPKYMQVIYTGMLGIFEDFKDNLINARGKDYCIDYAIEVFKEIVRAYQREAEYFHTGYVPSYDEYMENSIISGGYKMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 4 - protein, 548 aa, Artificial Sequence
  Synthetic Sequence
MASSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAEGYYWMMGAHFEPKFSLSRKFLARIFGITSLIDDTYDVYGTLEEVTLFTEAVERWDIEAVKDIPKYMQVIYTGMLGIFEDFKDNLINARGKDYCIDYAIEVFKEIVRAYQREAEYFHTGYVPSYDEYMENSIISGGYKMLIILMLIGRGEFELKETLDWASTIPEMVKASSLIARLIDDLQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 5 - protein, 548 aa, Artificial Sequence
  Synthetic Sequence
MASSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAEGYYWIMGAHFEPKFSLSRKFLARIFGITSLIDDTYDVYGTLEEVTLFTEAVERWDIEAVKDIPKYMQVIYTGMLGIFEDFKDNLINARGKDYCIDYAIEVFKEIVRAYQREAEYFHTGYVPSYDEYMENSIVSGGYKMLIILMLIGRGEFELKETLDWASTIPEMVKASSLIARLIDDVQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 6 - protein, 505 aa, Artificial Sequence
  Synthetic Sequence
MAWEYALIGLVVGIIIGAVAMRWYLKSYTSARRSQSNHLPRVPEVPGVPLLGNLLQLKEKKPYMTFTKWAATYGPIYSIKTGATSVVVVSSNEIAKEALVTRFQSISTRNLSKALKVLTADKQMVAMSDYDDYHKTVKRHILTAVLGPNAQKKHRIHRDIMMDNISTQLHEFVKNNPEQEEVDLRKIFQSELFGLAMRQALGKDVESLYVEDLKITMNRDEILQVLVVDPMMGAIDVDWRDFFPYLKWVPNKKFENTIQQMYIRREAVMKSLIKEQKKRIASGEKLNSYIDYLLSEAQTLTDQQLLMSLWEPIIESSDTTMVTTEWAMYELAKNPKLQDRLYRDIKSVCGSEKITEEHLSQLPYITAIFHETLRKHSPVPILPLRHVHEDTVLGGYHVPAGTELAVNIYGCNMDKNVWENPEEWNPERFMKENETIDFQKTMAFGGGKRVCAGSLQALLIASIGIGRMVQEFEWKLKDMTQEEVNTIGLTNQMLRPLRAIIKPRI

SEQ ID NO: 7 - protein, 508 aa, Artificial Sequence
  Synthetic Sequence
MAQDLRLILIIVGAIAIIALLVHGFWYLKSYTSARRSQSNHLPRVPEVPGVPLLGNLLQLKEKKPYMTFTKLAATYGPIYSIKTGATSVVVVSSNEIAKEALVTRFQSISTRNLPKALKVLTADKQMVAMSDYDDYHKTVKRHILTAVLGPNAQKKHRIHRDIMMDNVSTQLHAFVKNNPEQEEVDLRKIFQSELFGLAMRQALGKDVESLYVEDLKITMNRDEILQVLVVDPMRGAISVDWRDFFPYLKWIPNKKFDNTIQQMYIRREAVMKSLIKEQKKRIASGEKLNSYIDYLLSEAQTLTDQQLLMSLWEPIILSSDTTMVTTEWAMYELAKNPKLQERLYQDIKSVCGSEKITEEHLSQLPYLTAIFHETLRKHSPVPILPLRHVHEDTVLGGYHVPAGTELAVNIYGCNMDKNVWENPEEWNPERFMKENETIDFQKTMAFGGGRRVCAGSLQALLIASIGIGRMVQEFEWKLKDMTQEEVDTIGLTNHMAKPLRAIIKPRI

SEQ ID NO: 8 - protein, 508 aa, Artificial Sequence
  Synthetic Sequence
MAQDLRLILIIVGAIAIIALLVHGFWYLKSYTSARRSQSNHLPRVPEVPGVPLLGNLLQLKEKKPYMTFTKLAATYGPIYSIKTGATSVVVVSSNEIAKEALVTRFQSISTRNLPKALKVLTADKQMVAMSDYDDYHKTVKRHILTAVLGPNAQKKHRIHRDIMMDNVSTQLHAFVKNNPEQEEVDLRKIFQSELFGLAMRQALGKDVESLYVEDLKITMNRDEILQVLVVDPMRGAASVDWRDFFPYLKWIPNKKFDNTIQQMYIRREAVMKSLIKEQKKRIASGEKLNSYIDYLLSEAQTLTDQQLLMSLWEPIILSTDTTMVTTEWAMYELAKNPKLQERLYQDIKSVCGSEKITEEHLSQLPYLTAIFHETLRKHSPVPILPLRHVHEDTVLGGYHVPAGTELAVNIYGCNMDKNVWENPEEWNPERFMKENETIDFQKTMAFGGGRRVCAGSLQALLIASIGIGRMVQEFEWKLKDMTQEEVDTIGLTNHMAKPLRAIIKPRI

SEQ ID NO: 9 - protein, 508 aa, Artificial Sequence
  Synthetic Sequence
MAQDLRLILIIVGAIAIIALLVHGFWYLKSYTSARRSQSNHLPRVPEVPGVPLLGNLLQLKEKKPYMTFTKLAATYGPIYSIKTGATSVVVVSSNEIAKEALVTRFQSISTRNLPKALKVLTADKQMVAMSDYDDYHKTVKRHILTAVLGPNAQKKHRIHRDIMMDNVSTQLHAFVKNNPEQEEVDLRKIFQSELFGLAMRQALGKDVESLYVEDLKITMNRDEILQVLVVDPMRGAASVDWRDFFPYLKWIPNKKFDNTIQQMYIRREAVMKSLIKEQKKRIASGEKLNSYIDYLLSEAQTLTDQQLLMSLWEPIIASSDTTMVTTEWAMYELAKNPKLQERLYQDIKSVCGSEKITEEHLSQLPYLTATFHETLRKHSPVPILPLRHVHEDTVLGGYHVPAGTELAVNIYGCNMDKNVWENPEEWNPERFMKENETIDFQKTMAFGGGRRVCAGSLQALLIASIGIGRMVQEFEWKLKDMTQEEVDTGGLTNHMAKPLRAIIKPRI

SEQ ID NO: 10 - protein, 277 aa, Rhodococcus erythropolis
MARVEGQVALITGAARGQGRSHAIKLAEEGADVILVDVPNDVVDIGYPLGTADELDQTAKDVENLGRKAIVIHADVRDLESLTAEVDRAVSTLGRLDIVSANAGIASVPFLSHDIPDNTWRQMIDINLTGVWHTAKVAVPHILAGERGGSIVLTSSAAGLKGYAQISHYSAAKHGVVGLMRSLALELAPHRVRVNSLHPTQVNTPMIQNEGTYRIFSPDLENPTREDFEIASTTTNALPIPWVESVDVSNALLFLVSEDARYITGAAIPVDAGTTLK

SEQ ID NO: 11 - protein, 280 aa, Citrus sinensis
MATPPISSLISQRLLGKVALVTGGASGIGEGIVRLFHRHGAKVCFVDVQDELGYRLQESLVGDKDSNIFYSHCDVTVEDDVRRAVDLTVTKFGTLDIMVNNAGISGTPSSDIRNVDVSEFEKVFDINVKGVFMGMKYAASVMIPRKQGSIISLGSVGSVIGGIGPHHYISSKHAVVGLTRSIAAELGQHGIRVNCVSPYAVPTNLAVAHLPEDERTEDMFTGFREFAKKNANLQGVELTVEDVANAVLFLASEDARYISGDNLIVDGGFTRVNHSFRVFR

SEQ ID NO: 12 - protein, 262 aa, Citrus sinensis
MSKPRLQGKVAIIMGAASGIGEATAKLFAEHGAFVIIADIQDELGNQVVSSIGPEKASYRHCDVRDEKQVEETVAYAIEKYGSLDIMYSNAGVAGPVGTILDLDMAQFDRTIATNLAGSVMAVKYAARVMVANKIRGSIICTTSTASTVGGSGPHAYTISKHGLLGLVRSAASELGKHGIRVNCVSPFGVATPFSAGTINDVEGFVCKVANLKGIVLKAKHVAEAALFLASDESAYVSGHDLVVDGGFTAVTNVMSMLEGHG

SEQ ID NO: 13 - protein, 263 aa, Citrus sinensis
MSNPRMEGKVALITGAASGIGEAAVRLFAEHGAFVVAADVQDELGHQVAASVGTDQVCYHHCDVRDEKQVEETVRYTLEKYGKLDVLFSNAGIMGPLTGILELDLTGFGNTMATNVCGVAATIKHAARAMVDKNIRGSIICTTSVASSLGGTAPHAYTTSKHALVGLVRTACSELGAYGIRVNCISPFGVATPLSCTAYNLRPDEVEANSCALANLKGIVLKAKHIAEAALFLASDESAYISGHNLAVDGGFTVVNHSSSSAT

SEQ ID NO: 14 - protein, 278 aa, Citrus sinensis
MTTAGSRDSPLVAQRLLGKVALVTGGATGIGESIVRLFHKHGAKVCVVDINDDLGQHLCQTLGPTTRFIHGDVAIEDDVSRAVDFTVANFGTLDIMVNNAGMGGPPCPDIREFPISTFEKVFDINTKGTFIGMKHAARVMIPSKKGSIVSISSVTSAIGGAGPHAYTASKHAVLGLTKSVAAELGQHGIRVNCVSPYAILTNLALAHLHEDERTDDARAGFRAFIGKNANLQGVDLVEDDVANAVLFLASDDARYISGDNLFVDGGFTCTNHSLRVFR

SEQ ID NO: 15 - protein, 280 aa, Vitis vinifera
MAATSIDNSPLPSQRLLGKVALVTGGATGIGESIVRLFLKQGAKVCIVDVQDDLGQKLCDTLGGDPNVSFFHCDVTIEDDVCHAVDFTVTKFGTLDIMVNNAGMAGPPCSDIRNVEVSMFEKVFDVNVKGVFLGMKHAARIMIPLKKGTIISLCSVSSAIAGVGPHAYTGSKCAVAGLTQSVAAEMGGHGIRVNCISPYAIATGLALAHLPEDERTEDAMAGFRAFVGKNANLQGVELTVDDVAHAAVFLASDEARYISGLNLMLDGGFSCTNHSLRVFR

SEQ ID NO: 16 - protein, 279 aa, Vitis vinifera
MSTASSGDVSLLSQRLVGKVALITGGATGIGESIARLFYRHGAKVCIVDIQDNPGQNLCRELGTDDACFFHCDVSIEIDVIRAVDFVVNRFGKLDIMVNNAGIADPPCPDIRNTDLSIFEKVFDVNVKGTFQCMKHAARVMVPQKKGSIISLTSVASVIGGAGPHAYTGSKHAVLGLTKSVAAELGLHGIRVNCVSPYAVPTGMPLAHLPESEKTEDAMMGMRAFVGRNANLQGIELTVDDVANSVVFLASDEARYVSGLNLMLDGGFSCVNHSLRVFR

SEQ ID NO: 17 - protein, 280 aa, Citrus sinensis
MSNSNSTDSSPAVQRLVGRVALITGGATGIGESTVRLFHKHGAKVCIADVQDNLGQQVCQSLGGEPDTFFCHCDVTKEEDVCSAVDLTVEKFGTLDIMVNNAGISGAPCPDIREADLSEFEKVFDINVKGVFHGMKHAARIMIPQTKGTIISICSVAGAIGGLGPHAYTGSKHAVLGLNKNVAAELGKYGIRVNCVSPYAVATGLALAHLPEEERTEDAMVGFRNFVARNANMQGTELTANDVANAVLFLASDEARYISGTNLMVDGGFTSVNHSLRVFR

SEQ ID NO: 18 - protein, 282 aa, Brachypodium distachyon
MSAAAAVSSSSSPRLEGKVALVTGGASGIGEAIVRLFRQHGAKVCIADVQDEAGQQVRDSLGDDAGTDVLFVHCDVTVEEDVSRAVDAAAEKFGTLDIMVNNAGITGDKVTDIRNLDFAEVRKVFDINVHGMLLGMKHAARVMIPGKKGSIVSLASVASVMGGMGPHAYTASKHAVVGLTKSVALELGKHGIRVNCVSPYAVPTALSMPHLPQGEHKGDAVRDFLAFVGGEANLKGVDLLPKDVAQAVLYLASDEARYISALNLVVDGGFTSVNPNLKAFED

SEQ ID NO: 19 - protein, 267 aa, Zingiber zerumbet
MRLEGKVALVTGGASGIGESIARLFIEHGAKICIVDVQDELGQQVSQRLGGDPHACYFHCDVTVEDDVRRAVDFTAEKYGTIDIMVNNAGITGDKVIDIRDADFNEFKKVFDINVNGVFLGMKHAARIMIPKMKGSIVSLASVSSVIAGAGPHGYTGAKHAVVGLTKSVAAELGRHGIRVNCVSPYAVPTRLSMPYLPESEMQEDALRGFLTFVRSNANLKGVDLMPNDVAEAVLYLATEESKYVSGLNLVIDGGFSIANHTLQVFE

SEQ ID NO: 20 - protein, 709 aa, Camtotheca acuminta
MAQSSSVKVSTFDLMSAILRGRSMDQTNVSFESGESPALAMLIENRELVMILTTSVAVLIGCFVVLLWRRSSGKSGKVTEPPKPLMVKTEPEPEVDDGKKKVSIFYGTQTGTAEGFAKALAEEAKVRYEKASFKVIDLDDYAADDEEYEEKLKKETLTFFFLATYGDGEPTDNAARFYKWFMEGKERGDWLKNLHYGVFGLGNRQYEHFNRIAKVVDDTIAEQGGKRLIPVGLGDDDQCIEDDFAAWRELLWPELDQLLQDEDGTTVATPYTAAVLEYRVVFHDSPDASLLDKSFSKSNGHAVHDAQHPCRANVAVRRELHTPASDRSCTHLEFDISGTGLVYETGDHVGVYCENLIEVVEEAEMLLGLSPDTFFSIHTDKEDGTPLSGSSLPPPFPPCTLRRALTQYADLLSSPKKSSLLALAAHCSDPSEADRLRHLASPSGKDEYAQWVVASQRSLLEVMAEFPSAKPPIGAFFAGVAPRLQPRYYSISSSPRMAPSRIHVTCALVFEKTPVGRIHKGVCSTWMKNAVPLDESRDCSWAPIFVRQSNFKLPADTKVPVLMIGPGTGLAPFRGFLQERLALKEAGAELGPAILFFGCRNRQMDYIYEDELNNFVETGALSELIVAFSREGPKKEYVQHKMMEKASDIWNMISQEGYIYVCGDAKGMARDVHRTLHTIVQEQGSLDSSKTESMVKNLQMNGRYLRDVW

SEQ ID NO: 21 - protein, 711 aa, Stevia rebaudiana
MAQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 22 - protein, 693 aa, Arabidopsis thaliana
MATSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 23 - protein, 712 aa, Arabidopsis thaliana
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 24 - protein, 713 aa, Arabidopsis thaliana
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 25 - protein, 708 aa, Stevia rebaudiana
MAQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYFSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 26 - protein, 705 aa, Artemisia annua
MAQSTTSVKLSPFDLMTALLNGKVSFDTSNTSDTNIPLAVFMENRELLMILTTSVAVLIGCVVVLVWRRSSSAAKKAAESPVIVVPKKVTEDEVDDGRKKVTVFFGTQTGTAEGFAKALVEEAKARYEKAVFKVIDLDDYAAEDDEYEEKLKKESLAFFFLATYGDGEPTDNAARFYKWFTEGEEKGEWLDKLQYAVFGLGNRQYEHFNKIAKVVDEKLVEQGAKRLVPVGMGDDDQCIEDDFTAWKELVWPELDQLLRDEDDTSVATPYTAAVAEYRVVFHDKPETYDQDQLTNGHAVHDAQHPCRSNVAVKKELHSPLSDRSCTHLEFDISNTGLSYETGDHVGVYVENLSEVVDEAEKLIGLPPHTYFSVHADNEDGTPLGGASLPPPFPPCTLRKALASYADVLSSPKKSALLALAAHATDSTEADRLKFLASPAGKDEYAQWIVASHRSLLEVMEAFPSAKPPLGVFFASVAPRLQPRYYSISSSPRFAPNRIHVTCALVYEQTPSGRVHKGVCSTWMKNAVPMTESQDCSWAPIYVRTSNFRLPSDPKVPVIMIGPGTGLAPFRGFLQERLAQKEAGTELGTAILFFGCRNRKVDFIYEDELNNFVETGALSELVTAFSREGATKEYVQHKMTQKASDIWNLLSEGAYLYVCGDAKGMAKDVHRTLHTIVQEQGSLDSSKAELYVKNLQMAGRYLRDVW

SEQ ID NO: 27 - protein, 706 aa, Pelargonium graveolens
MAQSSSGSMSPFDFMTAIIKGKMEPSNASLGAAGEVTAMILDNRELVMILTTSIAVLIGCVVVFIWRRSSSQTPTAVQPLKPLLAKETESEVDDGKQKVTIFFGTQTGTAEGFAKALADEAKARYDKVTFKVVDLDDYAADDEEYEEKLKKETLAFFFLATYGDGEPTDNAARFYKWFLEGKERGEWLQNLKFGVFGLGNRQYEHFNKIAIVVDEILAEQGGKRLISVGLGDDDQCIEDDFTAWRESLWPELDQLLRDEDDTTVSTPYTAAVLEYRVVFHDPADAPTLEKSYSNANGHSVVDAQHPLRANVAVRRELHTPASDRSCTHLEFDISGTGIAYETGDHVGVYCENLAETVEEALELLGLSPDTYFSVHADKEDGTPLSGSSLPPPFPPCTLRTALTLHADLLSSPKKSALLALAAHASDPTEADRLRHLASPAGKDEYAQWIVASQRSLLEVMAEFPSAKPPLGVFFASVAPRLQPRYYSISSSPRIAPSRIHVTCALVYEKTPTGRVHKGVCSTWMKNSVPSEKSDECSWAPIFVRQSNFKLPADAKVPIIMIGPGTGLAPFRGFLQERLALKEAGTELGPSILFFGCRNSKMDYIYEDELDNFVQNGALSELVLAFSREGPTKEYVQHKMMEKASDIWNLISQGAYLYVCGDAKGMARDVHRTLHTIAQEQGSLDSSKAESMVKNLQMSGRYLRDVW

SEQ ID NO: 28 - protein, 548 aa, Artificial Sequence
  Synthetic Sequence
MASSAKLGSASEDVSRRDANYHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAEGYYWIMGAHFEPKFSLSRKFLARIFGIVSLIDDTYDVYGTLEEVTLFTEAVERWDITAVKDIPKYMQVIYTGMLGIFEDFKDNLINARGKDYCIDYAIEVFKEIVRAYQREAEYFHTGYVPSYDEYMENSIVSGGYKMLIILMLIGRGEFELKETLDWASTIPEMVKASSLIARLIDDVQTYKAEEERGETVSAVRCYMREFGVSEEQACKKMREMIEIEWKRLNKTTLEADEISSSVVIPSLNFTRVLEVMYDKGDGYSDSQGVTKDRIAALLRHAIEI

SEQ ID NO: 29 - protein, 508 aa, Artificial Sequence
  Synthetic Sequence
MAQDLRLILIIVGAIAIIALLVHGFWYLKSYTSARRSQSNHLPRVPEVPGVPLLGNLLQLKEKKPYMTFTKLAATYGPIYSIKTGATSVVVVSSNEIAKEALVTRFQSISTRNLPKALKVLTADKQMVAMSDYDDYHKTVKRHILTAVLGPNAQKKHRIHRDIMMDNVSTQLHAFVKNNPEQEEVDLRKIFQSELFGLAMRQALGKDVESLYVEDLKITMNRDEILQVLVVDPMRGAASVDWRDFFPYLKWIPNKKFDNTIQQMYIRREAVMKSLIKEQKKRIASGEKLNSYIDYLLSEAQTLTDQQLLMSLWEPIIASSDTTMVTTEWAMYELAKNPKLQERLYQDIKSVCGSEKITEEHLSQLPYLTATFHETLRKHSPVPILPLRHVHEDTVLGGYHVPAGTELAVNIYGCNMDKNVWENPEEWNPERFMKENETIDFQKTMAFGGGRRVCAGSLQALLIASIGIGRMVQEFEWKLKDMTQEEVDQGGLTNSMAKPLRAIIKPRI

SEQ ID NO: 30 - protein, 508 aa, Artificial Sequence
  Synthetic Sequence
MAQDLRLILIIVGAIAIIALLVHGFWYLKSYTSARRSQSNHLPRVPEVPGVPLLGNLLQLKEKKPYMTFTKLAATYGPIYSIKTGATSVVVVSSNEIAKEALVTRFQSISTRNLPKALKVLTADKQMVAMSDYDDYHKTVKRHILTAVLGPNAQKKHRIHRDIMMDNVSTQLHAFVKNNPEQEEVDLRKIFQSELFGLAMRQALGKDVESLYVEDLKITMNRDEILQVLVVDPMRGAASVDWRDFFPYLKWIPNKKFDNTIQQMYIRREAVMKSLIKEQKKRIASGEKLNSYIDYLLSEAQTLTDQQLLMSLWEPIIASSDTTMVTTEWAMYELAKNPKLQERLYQDIKSVCGSEKITEEHLSQLPYLTATFHETLRKHSPVPILPLRHVHEDTVLGGYHVPAGTELAVNIYGCNMDKNVWENPEEWNPERFMKENETINFQKTMAFGGGRRVCAGSLQALLIASIGIGRMVQEFEWKLKDMNQEEVDQGGLTNSMAKPLRAIIKPRI

SEQ ID NO: 31 - protein, 548 aa, Artificial Sequence
  Synthetic Sequence
MASSAKLGSASEDVSRRDANFHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAESYYWIMGAHFEPKFSLSRKFLARIFGIVSLIDDTYDVYGTLEEVTLFTEAVERWDITAVKDIPKYMQVIYTGMLGIFEDFKDNLINARGKDYCIDYAIEVFKEIVRAYQREAEYFHTGYVPSYDEYMENSIVSGGYKMLIILMLIGRGEFELKETLDWASTIPEMVKASSLIARLIDDVVTYKAEEERGETVSAVRCYMREFGVSEEQACKKMREMIEIEWKRLNKTTLEADEISSSVVIPSLNFTRVLEVMYDKGDGYSDSQGVTKDRIAALLRHPIEI

SEQ ID NO: 32 - protein, 548 aa, Artificial Sequence
  Synthetic Sequence
MASSAKLGSASEDVSRRDANFHPTVWGDFFLTHSSNFLENNDSILEKHEELKQEVRNLLVVETSDLPSKIQLTDEIIRLGVGYHFETEIKAQLEKLHDHQLHLNFDLLTTSVWFRLLRGHGFSISSDVFKRFKNTKGEFETEDARTLWCLYEATHLRVDGEDILEEAIQFSRKKLEALLPELSFPLNECVRDALHIPYHRNVQRLAARQYIPQYDAEPTKIESLSLFAKIDFNMLQALHQRELREASRWWKEFDFPSKLPYARDRIAESYYWIMGAHFEPKFSLSRKFLARIFGIVSLIDDTYDVYGTLEEVTLFTEAVERWDITAVKDIPKYMQVIYTGMLGIFEDFKDNLINARGKDYCIDYAIEVFKEIVRAYQREAEYFHTGYVPSYDEYMENSIVSGGYKMLIILMLIGRGEFELKETLDWASTIPEMVKASSLIARLIDDVQTYKAEEERGETVSAVRCYMREFGVSEEQACKKMREMIEDEWKRLNKTTLEADEISSSVVIPSLNFTRVLEVMYDKGDGYSDSQGVTKDRIAALLRHPIEI

SEQ ID NO: 33 - protein, 508 aa, Artificial Sequence
  Synthetic Sequence
MAQDLRLILIIVGAIAIIALLVHGFWYLKSYTSARRSQSNHLPRVPEVPGVPLLGNLLQLKEKKPYMTFTKLAATYGPIYSIKTGATSVVVVSSNEIAKEALVTRFQSISTRNLPKALKVLTADKQMVAMSDYDDYHKTVKRHILTAVLGPNAQKKHRIHRDIMMDNVSTQLHAFVKNNPEQEAVDLRKIFQSELFGLAMRQALGKDVESLYVEDLKITMNRDEILQVLVVDPMRGAASVDWRDFFPYLKWIPNKKFDNTIQQMYIRREAVMKSLIKEQKKRIASGEKLNSYIDYLLSEAQTLTDQQLLMSLWEPIIASSDTTMVTTEWAMYELAKNPKLQERLYQDIKSVCGSEKITEEHLSQLPYLTATFHETLRKHSPVPILPLRYVHEDTVLGGYHVPAGTELAVNIYGCNMDKNVWENPEEWNPERFMKENETINFQKTMAFGGGRRVCAGSLQALLIASIGIGRMVQEFEWKLKDMNQEEVDQGGLTNSMAKPLHAIIKPRI

SEQ ID NO: 34 - protein, 709 aa, Artificial Sequence
  Synthetic Sequence
MAQSESRSMKVSPLELMSAIIRKAMDPSRESSESVREVATLILENREFVMILTTLLAVLIGCVVVLVWKRSSGQKAKPFEPPKQLIVKEPEPEVDDGKKKVTVFFGTQTGTAEGFAKALAEEAKARYEKATFRVVDLDDYAADDDEYEEKLKKETLAIFFLATYGDGEPTDNAARFYKWFSEGKEKGDWISNLQYAVFGLGNRQYEHFNKIAKVVDEQLAEQGGKRLVPVGLGDDDQCIEDDFSAWREALWPELDKLLRDDDDSTTVATPYTAAVLEYRVVFYDAADVSVEDKRWAFANGHAVYDAQHPCRANVAMRKELHTPASDRSCIHLEFDISGTGLTYETGDHVGVFCENLDETVEDAIRLIGLSPETYFSIHTDKDDGTPLGGSSLPPPFAPCTLRTALTQYADLLSSPKKSALVALAAHASDPAEADRLRHLSSPAGKDEYAQWIIASQRSLLEVMAEFPSAKPPLGVFFAAVAPRLQPRYYSISSSPRMAPSRIHVTCALVYDKTPTGRIHKGVCSTWMKNAVPLEESQACSWAPIYVRQSNFKLPTDSKLPIIMIGPGTGLAPFRGFLQERLALKEAGVELGHSILFFGCRNRKMDYIYEDELSNFAETGALSELIVAFSREGPTKEYVQHKMVDKASDIWNILSQGGYIYVCGDAKGMARDVHRTLHNIVQEQGSLDSSKAESMVKNLQMSGRYLRDVW

SEQ ID NO: 35 - protein, 689 aa, Artificial Sequence
  Synthetic Sequence
MAESLNGGSIDLSIPASMALLFENRELLMLLTTSIAILIGCVVVLVWRRSSSQGSAKSFEPPKLTISKIEPEEEVDDGKKKVTIFFGTQTGTAEGFAKAFAEEAKARYEKAKFKVIDLDDYAEDDDEYEAKLKKESLALFFLATYGDGEPTDNAARFYKWFSEGEEKDEWLKNLQYGVFGLGNRQYEHFNKIAKVVDDGLAEQGAKRLVPVGMGDDDQCIEDDFTAWRELAWPELDQLLLDKEDAAVATPYTAAVLEYRVVVHDQTDTSLLDRNLSTLNGHTVYDAQHPCRSNVAVKRELHTPASDRSCIHLEFDISHTGLSYETGDHVGVYCENLIEIVEEAERLLGIAPATYFSVHTDKEDGTPLSGGSLPPPFPPCTLRTALTRYADLLSSPKKSALLALAAHASDSSEADRLRFLASPAGKDEYAQWLVANQRSLLEVMAEFPSAKPPLGVFFASIAPRLQPRYYSISSSPRMAPSRIHVTCALVYEKTPTGRIHKGVCSTWMKNAVSLEENNDCSWAPIFVRQSNFKLPSDTKVPIIMIGPGTGLAPFRGFLQERLALKEAGAELGPAVLYFGCRNRKLDFIYEDELNNFVETGVISELVLAFSREGATKEYVQHKMSQKALEVWNLISQGAYIYVCGDAKGMARDVHRMLHTIAQEQGALDSSKAESLVKNLQMTGRYLRDVW

SEQ ID NO: 36 - protein, 709 aa, Artificial Sequence
  Synthetic Sequence
MAQSESRSMKVSTLELISAIIRKAMDPSQDSSESVKEVATLMMENREFVMIVTTSIAVLIGCVVVLVWKRSSSQKVKSFEPPKQLIVKEPEPEVEDGKKKVTVFFGTQTGTAEGFAKALAEEAKARYEKATFRVVDLDDYAADDDEYEEKLRKETITIFFLATYGDGEPTDNAARFYKWFSEGKEKGEWISNLQYAVFGLGNRQYEHFNKIALVVDEQLAEQGGKRLVPVGLGDDDQCIEDDFTAWREALWPELDKLLRDEDDSTTASTPYTAAVLEYRVVFYDAADVPGGDKRWSLANGHSVYDAQHPCRSNVAVRKELHTPASDRSCTHLEFDISGTGLTYETGDHVGVFCENLDEVVEEALRLLGLSPETYFSIHADKEDGTPLTGSSLPPLFAPCTLRTALTQYADLLSSPKKSALVALAAHASDPAEADRLRHLSSPAGKDEYSQWIIASQRSLLEVMAEFPSARPPLGVFFAAVAPRLQPRYYSISSSPRMAPSRINVTCALVYDKTPTGRIHKGVCSTWMKSAVSLEESQACSWAPIYVRQSNFKLPTDSKLPIIMIGPGTGLAPFRGFLQERLALKEAGVELAHSILFFGCRNRNMDYIYEYELNNFVETGALSELIVAFSREGPSKEYVQHKMVEKASEIWNLLSQGAYIYVCGDAKGMARDVHRTLHNIVQEQGSLDSSKAESMVKNLQMSGRYLRDVW